SEQUENCE LISTING (30 sequences)

SEQ ID NO: 1 - protein, 6 aa, Artificial sequence
  Peptide
  MISC_FEATURE (1)..(1): Xaa is a member independently selected from Ala, Val, Leu, Ile, Phe and Pro
  MISC_FEATURE (2)..(2): Xaa is a member independently selected from Lys, Arg and His
  MISC_FEATURE (3)..(3): Xaa is a member independently selected from Lys, Arg and His
  MISC_FEATURE (4)..(4): Xaa is a member independently selected from Ala, Val, Leu, Ile, Phe and Pro
  MISC_FEATURE (5)..(5): Xaa is a member independently selected from Lys, Arg and His
  MISC_FEATURE (6)..(6): Xaa is a member independently selected from Ala, Val, Leu, Ile, Phe and Pro
XXXXXX

SEQ ID NO: 2 - protein, 7 aa, Artificial sequence
  Peptide
FHRRIKA

SEQ ID NO: 3 - protein, 6 aa, Artificial sequence
  Peptide
PRRARV

SEQ ID NO: 4 - protein, 4 aa, Artificial sequence
  Peptide
REDV

SEQ ID NO: 5 - protein, 4 aa, Artificial sequence
  Peptide
DEGA

SEQ ID NO: 6 - protein, 5 aa, Artificial sequence
  Peptide
YIGSR

SEQ ID NO: 7 - protein, 5 aa, Artificial sequence
  Peptide
IKVAV

SEQ ID NO: 8 - protein, 5 aa, Artificial sequence
  Peptide
PHSRN

SEQ ID NO: 9 - protein, 15 aa, Artificial sequence
  bsp-RGD(15)
CGGNGEPRGDTYRAY

SEQ ID NO: 10 - protein, 22 aa, Artificial sequence
  bsp-RGD(15)-FITC
CGGNGEPRGDTYRAYKFITCGG

SEQ ID NO: 11 - protein, 15 aa, Artificial sequence
  bsp-RGE(15)
CGGNGEPRGETYRAY

SEQ ID NO: 12 - protein, 10 aa, Artificial sequence
  Heparin-binding adhesion peptide
CGGFHRRIKA

SEQ ID NO: 13 - protein, 5 aa, Artificial sequence
  Peptide
RGDYK

SEQ ID NO: 14 - protein, 9 aa, Artificial sequence
  Peptide
DYKDDDDKC

SEQ ID NO: 15 - protein, 19 aa, Artificial sequence
  lam-IKVAV(19) peptide
CSRARKQAASIKVAVSADR

SEQ ID NO: 16 - DNA, 20 bp, Artificial sequence
  Primer
gacctgcgaccttgagtcct

SEQ ID NO: 17 - DNA, 20 bp, Artificial sequence
  Primer
tctcctccttgaggctttgg

SEQ ID NO: 18 - DNA, 21 bp, Artificial sequence
  Hybridization TAQMAN oligo
tccttggagaggcaaatgcgc

SEQ ID NO: 19 - DNA, 23 bp, Artificial sequence
  Primer
gcatggatgagatggagttcacc

SEQ ID NO: 20 - DNA, 25 bp, Artificial sequence
  Primer
cgactcctcgtcgtcatcttcatac

SEQ ID NO: 21 - DNA, 20 bp, Artificial sequence
  Hybridization TAQMAN oligo
tgaacgacctggtgtctgag

SEQ ID NO: 22 - DNA, 20 bp, Artificial sequence
  Primer
gagctctctgggcaagtgga

SEQ ID NO: 23 - DNA, 20 bp, Artificial sequence
  Primer
ctcccaccgctgttgatttc

SEQ ID NO: 24 - DNA, 22 bp, Artificial sequence
  Hybridization TAQMAN oligo
aggacagtcagcagtgcctgca

SEQ ID NO: 25 - DNA, 21 bp, Artificial sequence
  Primer
gtaacccgttgaaccccattc

SEQ ID NO: 26 - DNA, 21 bp, Artificial sequence
  Primer
ccatccaatcggtagtagcga

SEQ ID NO: 27 - DNA, 22 bp, Artificial sequence
  Hybridization TAQMAN oligo
aagtgcgggtcataagcttgcg

SEQ ID NO: 28 - protein, 7 aa, Artificial sequence
  Peptide crosslinker
QPQGLAK

SEQ ID NO: 29 - protein, 9 aa, Artificial sequence
  Peptide crosslinker
GPLGLSLGK

SEQ ID NO: 30 - protein, 8 aa, Artificial sequence
  Peptide crosslinker
GPLGMHGK

(19) United States
(12) Patent Application Publication
(10) Pub. No.: US 2007/0026518 A1
(43) Pub. Date: Feb. 1, 2007

(54) Controlling stem cell destiny with tunable matrices

(75) Inventors: Kevin E. Healy, Moraga, CA (US); Beth Irwin, San Francisco, CA (US); Jacob Freas Pollock, Kensington, CA (US); David Schaffer, Pleasant Hill, CA (US); Krishanu Saha, Berkeley, CA (US); Ying Li, San Francisco, CA (US); Samuel Thomas Wall, San Francisco, CA (US)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: The Regents of the University of California, Oakland, CA (US)

(21) Appl. No.: 11/394,042

(22) Filed: Mar. 29, 2006

Related U.S. Application Data
(60) Provisional application No. 60/666,734, filed on Mar. 29, 2005.

(51) Int. Cl. C12N 5/08; C12N 5/00; C12N 5/02; C08G 63/91

(52) U.S. Cl. 435/325; 435/366; 435/404; 525/54.1

(57) ABSTRACT

The present invention provides a class of interpenetrating polymeric networks (IPNs) and semi-interpenetrating polymeric networks (sIPNs) which include a covalently grafted growth factor or differentiation factor for a stem cell.

### CROSS-REFERENCES TO RELATED APPLICATIONS

The present application claims priority to U.S. Provisional Patent Application No. 60/666,734, filed on Mar. 29, 2005, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was supported in part by grant number R01 AR47304 from the NIH/NIAMS; 5R21NS048248 from the NIH; a National Science Foundation Graduate Fellowship to J. Pollack and K. Saha; a National Defense Science Engineering Graduate Fellowship to Y. Li; and DOD ONR funds, Grant No.: N00014-01-08121. The Government may have rights in the subject matter disclosed herein.

### BACKGROUND OF THE INVENTION

Previously, p(NIPAAm) homopolymer, copolymer chains, crosslinked hydrogels and p(NIPAAm)-based sIPNs (and also IPNs, which consist of two cross-linked networks that are physically entangled within each other but are not chemically connected in any way) have been studied for use in a number of diverse applications including solute recovery, (Freitas et al., Chemical Engineering Science, 42:97-103 (1987)) solute delivery, (Hoffman et al., Journal of Controlled Release, 4:213-222 (1986); Vakkalanka et al., Journal of Biomaterials Science, Polymer Edition, 8:119-129 (1996)) cell adhesion and manipulation, (Okano et al., Journal of Biomedical Materials Research, 27:1243-1251 (1993)) bioseparations, (Monji et al., Applications in Biochemistry and Biotechnology, 14:107-120 (1987)) catalytic reaction control, (Park et al., Biotechnology Progress, 7:383-390 (1991)) microencapsulation of cells, (Shimizu et al., Artificial Organs, 20:1232-1237 (1996)) chromatography, (Lakhiari et al., Biochimica et Biophysica Acta, 1379:303-313 (1998)) development of a biohybrid artificial pancreas, (Vernon et al., Macromolecular Symposia, 109:155-167 (1996)) and cell growth for tissue regeneration (Stile et al., Biomacromolecules, 2:185-194 (2001); Stile et al., Macromolecules, 32:7370-7379 (1999)). The evolution of most of these applications was based on the unique phase behavior of p(NIPAAm) in aqueous media. The linear polymer chains (in the case of a sIPN) or the second network (in the case of an IPN) were added to the p(NIPAAm)-based hydrogels to change the swelling characteristics and/or the mechanical properties of the matrices. To our knowledge, there are no publications to date in which the polymer chains or the second network was modified with biomolecules to impart biological functionality to the sIPN or IPN.

Previous work has led to the development of injectable p(NIPAAm-co-AAc) hydrogels that demonstrated a phase transition below body temperature, during which the rigidity of the matrix significantly increased. During in vitro culture, these matrices supported bovine articular chondrocyte viability and promoted the formation of tissue with histoarchitecture similar to that of native articular cartilage. Furthermore, when the AAc groups in the p(NIPAAm-co-AAc) hydrogel were functionalized with peptides containing relevant sequences found in ECM macromolecules, the peptide-modified hydrogels supported rat calvarial osteoblast viability, spreading, and proliferation. However, the procedure used to functionalize the hydrogels with the peptide sequences adversely altered the volume change characteristics of the hydrogels, significantly limiting the clinical utility of these matrices.

In order to replace or repair damaged tissues in the human body, regenerative medicine requires reliable, specific sources of cells from which to implant or engineer ex vivo into tissue equivalents. Stem cells are a compelling source of both undifferentiated and differentiated cells. Harvests of stem cells, and generally all stem cell lines, are heterogeneous: many different cell types, ranging from very immature multipotent cells to terminally differentiated cells, are in the cell culture. Propagating and controlling this heterogeneous cell population has proven to be very difficult, involving a range of growth factors and protein substrates. Maturation of stem cells occurs by two processes: selection and/or instruction. In selection, stem cells change their phenotype by some process internal to the cell, and the environment surrounding the cell selects or determines which cells survive or propagate. In contrast, instructive mechanisms involve active signaling or communication from the environment to the stem cell to instruct which behavior or mature phenotype it should adopt or develop into. In either case, improved means of controlling the signaling environment of a stem cell are required to control the behavior and differentiation state of a stem cell culture.

A significant advance in the art of regenerative medicine could be realized with a matrix that can be tuned to provide an environment for cell culture that has the desired chemical and physical properties. A polymer such as a sIPN that can be functionalized to interact with cells on a molecular level, or to serve as a drug delivery vehicle while maintaining predictable and useful swelling properties provides such a matrix.

### BRIEF SUMMARY OF THE INVENTION

In a first aspect, the invention provides an interpenetrating polymer network comprising (a) a first cross-linked polymer; and (b) a second cross-linked polymer entangled within said first cross-linked polymer, wherein a member selected from said first cross-linked polymer and said second cross-linked polymer is covalently grafted to a ligand which promotes a member selected from stem cell adhesion to the network, stem cell growth, stem cell proliferation, stem cell self-renewal, stem cell differentiation, and combinations thereof.

In a second aspect, the invention provides a semi-interpenetrating polymer network comprising (a) a cross-linked polymer; and (b) a linear polymer entangled within said cross-linked polymer, wherein said linear polymer is covalently grafted to a ligand which promotes a member selected from stem cell adhesion to the network, stem cell growth, stem cell proliferation, stem cell self-renewal, stem cell differentiation, and combinations thereof.

In an exemplary embodiment, the ligand in the network is a member selected from amino acids, peptides, peptoids, proteins, nucleic acids, carbohydrates and combinations thereof. In an exemplary embodiment, the ligand is a nucleic acid, which is a member selected from plasmid DNA, messenger RNA, viral DNA, viral RNA, small oligonucleotide DNA, small oligonucleotide RNA, and small interfering RNA. In yet another exemplary embodiment, the ligand is a member selected from antibodies and cytokines. In still another exemplary embodiment, the ligand is an extracellular matrix protein, or a portion thereof. In one exemplary embodiment, the peptide comprises a sequence which is a member selected from RGD, XBBXBX, FHRRIKA, PRRARV, REDV, DEGA, YIGSR, IKVAV, PHSRN, KGD, and cyclic variants thereof. Each X is a member independently selected from glycine, alanine, valine, leucine, isoleucine, phenylalanine and proline, and each B is a member independently selected from lysine, arginine and histidine.

In an exemplary embodiment, the ligand in the network affects a stem cell which is a member selected from embryonic stem cells, adult marrow stem cells, adult neural stem cells, cord blood stem cells, adult skin stem cells, adult liver stem cells, adult olfactory stem cells, adult adipose-derived stem cells, adult hair follicle stem cells, adult skeletal muscle stem cells, adult myogenic muscle stem cells, satellite cells, mesenchymal stem cells and neural stem cells.

In an exemplary embodiment, the network further comprises a stem cell. In another exemplary embodiment, the stem cell is a member selected from embryonic stem cells, adult marrow stem cells, adult neural stem cells, cord blood stem cells, adult skin stem cells, adult liver stem cells, adult olfactory stem cells, adult adipose-derived stem cells, adult hair follicle stem cells, adult skeletal muscle stem cells, adult myogenic muscle stem cells, satellite cells, mesenchymal stem cells and neural stem cells.

In an exemplary embodiment, the network further comprises a molecule which is non-covalently entangled with the network. In an exemplary embodiment, the molecule is a member selected from peptides, morphogens, growth factors, hormones, small molecules and cytokines. In an exemplary embodiment, the molecule is a member selected from adhesion peptides from ECM molecules, laminin peptides, heparin sulfate proteoglycan binding peptides, heparan sulfate proteoglycan binding peptides, Hedgehog, Sonic Hedgehog, Shh, Wnt, bone morphogeneic proteins, Notch (1-4) ligands, Delta-like ligand 1, 3, and 4, Serrate/Jagged ligands 1 and 2, fibroblast growth factor, epidermal growth factor, platelet derived growth factor, Eph/Ephrin, Insulin, Insulin-like growth factor, vascular endothelial growth factor, neurotrophins, BDNF, NGF, NT-3/4, retinoic acid, forskolin, purmorphamine, dexamethasone, 17β-estradiol and metabolites thereof, 2-methoxyestradiol, cardiogenol, stem cell factor, granulocyte-macrophage colony-stimulating factor, granulocyte colony-stimulating factor, interleukins, IL-6, IL-11, cytokines, Flt3-1, Leukaemia inhibitory factor, transferrin, intercellular adhesion molecules, ICAM-1 (CD54), VCAM, NCAM, tumor necrosis factor alpha, HER-2, and stromal cell-derived factor-1 alpha.

In an exemplary embodiment, there is a cross link in at least one of the cross-linked polymers in the interpenetrating polymer network or the cross link in the cross-linked polymer of the semi-interpenetrating polymer network which is biodegradable.

In an exemplary embodiment, there is a cross link between said ligand and a member selected from said cross-linking polymer and said linear polymer, wherein said cross link is biodegradable.

In an exemplary embodiment, the cross link in the network is degraded by a member selected from an enzyme and hydrolysis. In an exemplary embodiment, the cross link is degraded by an enzyme, and said enzyme is a collagenase.

In another exemplary embodiment, the cross-linked polymer or said linear polymer of the network is non-fouling. In another exemplary embodiment, the non-fouling cross-linked polymer or linear polymer comprises a subunit which is a member selected from hyaluronic acid, acrylic acid, ethylene glycol, methacrylic acid, acrylamide, hydroxyethyl methacrylate, mannitol, maltose, taurine, betaine, modified celluloses, hydroxyethyl cellulose, ethyl cellulose, methyl cellulose, hydroxyethyl methyl cellulose, hydroxypropyl methyl cellulose, carboxymethyl cellulose, modified starches, hydrophobically modified starch, hydroxyethyl starch, hydroxypropyl starch, amylose, amylopectin, oxidized starch, and copolymers thereof.

In another exemplary embodiment, the linear polymer comprises a subunit functionalized with said ligand, said subunit is derived from a member selected from hyaluronic acid, acrylic acid, ethylene glycol, methacrylic acid, dimethylaminopropylacrylamide, 2-acrylamido-2-methylpropane sulfonic acid, hydroxyethyl methacrylate, mannitol, maltose, taurine, betaine and copolymers thereof. In another exemplary embodiment, the linear polymer is polyacrylic acid in which at least one acrylic acid subunit is functionalized with said ligand.

In another aspect, the invention provides a method for self-renewal of a stem cell population, said method comprising: adhering said stem cell population to a network of the invention under conditions appropriate to support the self-renewal.

In another aspect, the invention provides a method of differentiating a stem cell population, the method comprising: adhering said stem cell population to the network under conditions appropriate to support the differentiating.

In another aspect, the invention provides a method of detaching a stem cell from the network, said method comprising: adhering said stem cell to the network and inducing a lower critical solution temperature phase transition in said network; thereby detaching said stem cell from said network.

Other aspects, objects and advantages of the present invention will be apparent from the detailed description that follows.

### BRIEF DESCRIPTION OF THE DRAWINGS

FIG. 1. Diagram and Characterization of IPN. a) Schematic of interpenetrating polymer network (IPN) synthesis (not to scale). Sequential polymerization steps create an IPN that is swollen in aqueous media and conjugated to bioactive peptides.

b) & c) Representative results showing the thickness as well as the shear, loss, and complex shear moduli (G′, G″, and ≡G*| respectively, where G*=G′+iG″) from the Kelvin-Voigt modeling of the PBS swelling of the hydrogel. The initial hydration of the hydrogel surface from the dry state is shown, as well as swelling of the surface. Zero modulus represents the unmodified substrate. There was an increase in thickness and a decrease in all moduli for all surfaces with swelling. Note that the dry characteristics of the IPN are as follows: XPS peak intensity ratios (i.e., O/N and C/N) indicated IPN coating of the poly(styrene) substrate, while angle-resolved studies demonstrated that the pAAm and PEG/AAc networks were interpenetrating with a dry thickness of ˜3.5-4.4 nm. The dry thickness in ambient humidity (˜5 nm in 49±7% relative humidity) was slightly larger than that determined by angle-resolved XPS (data not shown).

d) Ligand density data (mean±s.d.) for bsp-RGD(15)—FITC: Data representing input concentrations from 0.0046 to 0.46 μM with respective densities of ≈0.5 to 18 pmol/cm².

FIG. 2 depicts the change in the Young's modulus (E) as the concentration of BIS is used in the polymerization of the AAm layer is varied. The E of the gels varied linearly from 0.23±0.09 kPa to 9.86±0.14 kPa, and the square of the correlation coefficient (R²) is 0.9735.

FIG. 3. Synthetic IPNs with RGD peptides support attachment, spreading, and proliferation of neural stem cells in a dose dependent manner.

a)-d) Bright field images of neural stem cells grown on top of IPNs or laminin-I in proliferating media conditions (1.2 nM basic fibroblast growth factor); e) Growth curves for proliferation of neural stem cells as assayed by a total nucleic acid stain. Data represent mean±standard deviation of 3-5 samples. Surfaces not in the same group (*, §, †, or ‡) were statistically different from one another (p<0.05; ANOVA between groups with Tukey-Kramer Honestly Significant Difference post-hoc test).

FIG. 4 Cell phenotype of immature and differentiated cells on synthetic RGD-modified IPNs. a) Immunofluorescent staining for the immature neuronal stem cell marker nestin (green) in cells proliferating on laminin or 21 pmol.cm⁻² bsp-RGD(15) modified IPNs (media conditions: 1.2 nM basic fibroblast growth factor). In all stained images, cell nuclei were stained with Sybergreen or DAPI (blue); b) Bright field images of neural stem cells on laminin or 21 pmol.cm⁻² RGD-modified hydrogels during neuronal differentiation (media conditions: 1 μM retinoic acid with 5 μM forskolin for six days); Cellular staining for c) the early neuronal marker microtubule associated protein 2ab (Map2ab, green) and d) the mature astrocyte marker glial fibrillary acidic protein (GFAP, red) on laminin or 21 pmol.cm⁻² RGD modified hydrogels during differentiation. Right-hand panels compare expression levels as measured by quantitative RT-PCR during proliferation and differentiation for lineage markers, Nestin, β-tubulin III, and GFAP. The box plots summarize the distribution of points, where the thick line signifies the median and the ends of the box are the 25th and 75th quartiles. Within each plot, levels not connected by same letter are significantly different (p <0.05; ANOVA between groups with Tukey-Kramer Honestly Significant Difference post-hoc test).

FIG. 5 In mixed peptide IPNs, bsp-RGD(15) peptide surface density controls phenotype. a) Bright field images of NSCs after six days in culture on IPNs with mixed peptide conjugation in differentiating (1 μM retinoic acid, 5 μM forskolin) media conditions. Surface density of peptide mixtures correspond to abscissa values directly below for bsp-RGD(15) plus lam-IKVAV(19) or bsp-RGE(15); b) Expression of early neuronal marker, β-Tubulin III, and astrocyte marker, glial fibrillary acidic protein (GFAP), of NSCs grown in differentiation media conditions as assayed by quantitative RT-PCR after six days. The box plots summarize the distribution of points, where the thick line signifies the median and the ends of the box are the 25th and 75th quartiles. Within each plot, levels not connected by same letter are significantly different (p<0.05; ANOVA between groups with Tukey-Kramer Honestly Significant Difference post-hoc test); c) Bright field images of NSCs after six days in culture on IPNs with 21 pmol.cm⁻² bsp-RGD(15) or lam-IKVAV(19) peptide conjugation in proliferating (1.2 nM bFGF) media conditions.

FIG. 6 is a scheme for preparing an exemplary modified linear polymer useful in a sIPN of the invention in which p(AAc) is the linear polymer chain and a synthetic peptide serves as the biomolecule. The —COO⁻ groups in the linear p(AAc) chains are reacted with one end of a heterobifunctional cross-linker. The other end of the cross-linker is then used to graft the biomolecule to the p(AAc) chains. In the figure, the solid lines represent the cross-linked polymer, the dashed lines represent the linear polymer, and the ovals represent the ligand.

FIG. 7 is a synthetic scheme for preparing a sIPN of the invention, which incorporates a biomolecule modified linear p(AAc) polymer. The modified p(AAc) chains are added to the polymerization formulation, and the p(NIPAAm-co-AAc) cross-linked network forms in the presence of the chains. Thus, the chains are physically entangled within the cross-linked network.

FIG. 8 Constant contour plot (left) and 3D empirical response surface (right) for cell proliferation (cells/cm²) on sIPNs as a function of G* and bsp-RGD(15) concentration after 5 d of culture. G* were measured at 37° C. at 5% strain at 1 Hz. bsp-RGD(15) was in the form of p(AAc)-g-bsp-RGD(15). The model had an R² value of 0.86 and indicated significant effects of [RGD] (p<0.05) and G* (p<0.05).

FIG. 9 hESCs cultured on sIPN of various RGD adhesion ligand concentrations. (A, B, C, D)=0, 45, 105, 150 μM, respectively. At 0 μM RGD concentration, very low hESC adhesion was observed. At 45 μM RGD concentration, colony morphology was highly variable, where some colonies exhibited tight borders while other did not. Qualitatively, hESCs cultured on sIPNs of higher RGD concentrations (105 and 150 μM) exhibited morphologies most similar to undifferentiated hESCs.

FIG. 10 Morphology and OCT-4 immunofluorescence of hESCs at Day 5. (A, B) hESCs cultured on MEFs exhibited small, tightly packed cells with distinct colony borders. (C, D) hESCs cultured on sIPN (|G*51 ˜70 Pa, 150 μM RGD) exhibited similar morphologies when compared to (A, B). (E, F) hESCs cultured on gelatin-adsorbed polystyrene exhibited morphologies of spontaneously differentiating cells, with spindle-shaped cells and indistinct colony borders. OCT-4 was present in some cells under all three conditions. However, note that in hESCs cultured on polystyrene (F), white arrows point to cells beyond the colony edge which were not positive for OCT-4.

FIG. 11 Morphology and SSEA-4 immunofluorescence of hESCs at Day 5. (A, B) hESCs cultured on MEFs. (C, D) hESCs cultured on sIPN (|G*| ˜70 Pa, 45 μM RGD). (E, F) hESCs cultured on gelatin-adsorbed polystyrene. SSEA-4 was present in colonies under all three conditions.

FIG. 12 Semi-IPNs support NSC proliferation but not differentiation. NSCs after 15 days on a p(NIPAAm-co-AAc) semi-IPNs with p(AAc)-g-RGD linear chains in either a, proliferating (1.2 nM bFGF) media conditions or b, differentiating (1 μM retinoic acid, 5 μM forskolin) media conditions. The semi-IPN properties were 60 μM polyacrylic acid-graft-RGD (p(AAc)-g-RGD) and the mean G* at 22° C. at 1 Hz was 24.40 Pa±2.0 (SD), and at 37° C. at 1 Hz was 87.40 Pa+2.1 (SD). Using a live/dead stain (calcein AM and Ethidium Homodimer), the green represents living cells while the red represent necrotic cells.

### DETAILED DESCRIPTION OF THE INVENTION

I. Abbreviations and Definitions

As used herein, “NIPAAm,” refers to “N-isopropylacrylamide.” The term “p(NIPAAm),” as used herein, refers to “poly(N-isopropylacrylamide).” As used herein, “BIS,” refers to “N,N′-methylenebisacrylamide.” The term, “AAc,” as used herein, refers to “acrylic acid.” The term, “p(AAC),” as used herein, refers to linear “poly(acrylic acid)” chains. The term, “p(NIPAAm-co-AAc),” as used herein, refers to a sIPN formed from poly(N-isopropylacrylamide) and a linear poly(acrylic acid). “AP,” as used herein, refers to “ammonium peroxydisulfate.” “TEMED,” as used herein, refers to “N,N,N′,N′-tetramethylethylenediamine.” “ECM,” as used herein, refers to “extracellular matrix.” The term “sIPN,” as used herein, refers to “semi-interpenetrating polymer network.” “IPN,” refers to an “inter-penetrating polymer network.” The term “EMCH,” as used herein, refers to “N-ε-(maleimidocaproic acid)hydrazide.” The term “RGD peptide” refers to a peptide that includes the three amino acid motif RGD.

“Peptide” refers to a polymer in which the monomers are amino acids and are joined together through amide bonds, alternatively referred to as a polypeptide. Additionally, unnatural amino acids, for example, β-alanine, phenylglycine and homoarginine are also included. Amino acids that are not gene-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer. In addition, other peptidomimetics are also useful in the present invention. As used herein, “peptide” refers to both glycosylated and unglycosylated peptides. Also included are petides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a fluorophore (e.g., quantum dot) or another moiety.

“Antibody,” as used herein, generally refers to a polypeptide comprising a framework region from an immunoglobulin or fragments or immunoconjugates thereof that specifically binds and recognizes an antigen. The recognized immunoglobulins include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

As used herein, “pharmaceutically acceptable carrier” includes any material, which when combined with the conjugate retains the conjugates' activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, intranasal or subcutaneous administration, or the implantation of a slow-release device e.g., a mini-osmotic pump, to the subject.

As used herein, the term “copolymer” describes a polymer which contains more than one type of subunit. The term encompasses polymer which include two, three, four, five, or six types of subunits.

As used herein, the term “essentially constant” refers to a second value which has only a small difference between a first, originally measured value. For example, a biochemical property, such as ligand density, is essentially constant between two sIPNs if the difference between the ligand density values in these sIPNs is 5% or less.

The term “isolated” refers to a material that is substantially or essentially free from components, which are used to produce the material. The lower end of the range of purity for the polymer networks is about 60%, about 70% or about 80% and the upper end of the range of purity is about 70%, about 80%, about 90% or more than about 90%.

“Hydrogel” refers to a water-insoluble and water-swellable cross-linked polymer that is capable of absorbing at least 3 times, preferably at least 10 times, its own weight of a liquid. “Hydrogel” and “thermo-responsive polymer” are used interchangeably herein.

The term “attached,” as used herein encompasses interaction including, but not limited to, covalent bonding, ionic bonding, chemisorption, physisorption and combinations thereof.

The term “biomolecule” or “bioorganic molecule” refers to an organic molecule typically made by living organisms. This includes, for example, molecules comprising nucleotides, amino acids, sugars, fatty acids, steroids, nucleic acids, polypeptides, peptides, peptide fragments, carbohydrates, lipids, and combinations of these (e.g., glycoproteins, ribonucleoproteins, lipoproteins, or the like).

“RGD” peptides refer to peptides containing the arginine-glycine-aspartate (RGD) motif modulate cell adhesion.

“Small molecule,” refers to species that are less than 1 kD in molecular weight, preferably, less than 600 D.

The term “autologous cells”, as used herein, refers to cells which are person's own genetically identical cells.

The term “heterologous cells”, as used herein, refers to cells which are not person's own and are genetically different cells.

The term “network”, as used herein, refers to an interpenetrating polymer network (IPN), a semi-interpenetrating polymer network (sIPN), or both. These IPNs and sIPNs are functionalized with a ligand as described herein.

The term “stem cells”, as used herein, refers to cells capable of differentiation into other cell types, including those having a particular, specialized function (i.e., terminally differentiated cells, such as erythrocytes, macrophages, etc.). Stem cells can be defined according to their source (adult/somatic stem cells, embryonic stem cells), or according to their potency (totipotent, pluripotent, multipotent and unipotent).

The term “unipotent”, as used herein, refers to cells that can produce only one cell type, but have the property of self-renewal which distinguishes them from non-stem cells.

The term, “multipotent”, or “progenitor”, as used herein, refers to cells which can give rise to any one of several different terminally differentiated cell types. These different cell types are usually closely related (e.g. blood cells such as red blood cells, white blood cells and platelets). For example, mesenchymal stem cells (also known as marrow stromal cells) are multipotent cells, and are capable of forming osteoblasts, chondrocytes, myocytes, adipocytes, neuronal cells, and β-pancreatic islets cells.

The term “pluripotent”, as used herein, refers to cells that give rise to some or many, but not all, of the cell types of an organism. Pluripotent stem cells are able to differentiate into any cell type in the body of a mature organism, although without reprogramming they are unable to de-differentiate into the cells from which they were derived. As will be appreciated, “multipotent”/progenitor cells (e.g., neural stem cells) have a more narrow differentiation potential than do pluripotent stem cells. Another class of cells even more primitive (i.e., uncommitted to a particular differentiation fate) than pluripotent stem cells are the so-called “totipotent” stem cells.

The term “totipotent”, as used herein, refers to fertilized oocytes, as well as cells produced by the first few divisions of the fertilized egg cell (e.g., embryos at the two and four cell stages of development). Totipotent cells have the ability to differentiate into any type of cell of the particular species. For example, a single totipotent stem cell could give rise to a complete animal, as well as to any of the myriad of cell types found in the particular species (e.g., humans). In this specification, pluripotent and totipotent cells, as well as cells with the potential for differentiation into a complete organ or tissue, are referred as “primordial” stem cells.

The term “dedifferentiation”, as used herein, refers to the return of a cell to a less specialized state. After dedifferentiation, such a cell will have the capacity to differentiate into more or different cell types than was possible prior to re-programming. The process of reverse differentiation (i.e., de-differentiation) is likely more complicated than differentiation and requires “re-programming” the cell to become more primitive. An example of dedifferentiation is the conversion of a myogenic progenitor cell, such as early primary myoblast, to a muscle stem cell or satellite cell.

The term “anti-aging environment”, as used herein, is an environment which will cause a cell to dedifferentiate, or to maintain its current state of differentiation. For example, in an anti-aging environment, a myogenic progenitor cell would either maintain its current state of differentiation, or it would dedifferentiate into a satellite cell.

A “normal” stem cell refers to a stem cell (or its progeny) that does not exhibit an aberrant phenotype or have an aberrant genotype, and thus can give rise to the full range of cells that be derived from such a stem cell. In the context of a totipotent stem cell, for example, the cell could give rise to, for example, an entire, normal animal that is healthy. In contrast, an “abnormal” stem cell refers to a stem cell that is not normal, due, for example, to one or more mutations or genetic modifications or pathogens. Thus, abnormal stem cells differ from normal stem cells.

A “growth environment” is an environment in which stem cells will proliferate in vitro. Features of the environment include the medium in which the cells are cultured, and a supporting structure (such as a substrate on a solid surface) if present.

“Growth factor” refers to a substance that is effective to promote the growth of stem cells and which, unless added to the culture medium as a supplement, is not otherwise a component of the basal medium. Put another way, a growth factor is a molecule that is not secreted by cells being cultured (including any feeder cells, if present) or, if secreted by cells in the culture medium, is not secreted in an amount sufficient to achieve the result obtained by adding the growth factor exogenously. Growth factors include, but are not limited to, basic fibroblast growth factor (bFGF), acidic fibroblast growth factor (aFGF), epidermal growth factor (EGF), insulin-like growth factor-I (IGF-I), insulin-like growth factor-II (IGF-II), platelet-derived growth factor-AB (PDGF), and vascular endothelial cell growth factor (VEGF), activin-A, and bone morphogenic proteins (BMPs), insulin, cytokines, chemokines, morphogents, neutralizing antibodies, other proteins, and small molecules.

The term “differentiation factor”, as used herein, refers to a molecule that induces a stem cell to commit to a particular specialized cell type.

“Extracellular matrix” or “matrix” refers to one or more substances that provide substantially the same conditions for supporting cell growth as provided by an extracellular matrix synthesized by feeder cells. The matrix may be provided on a substrate. Alternatively, the component(s) comprising the matrix may be provided in solution. Components of an extracellular matrix can include laminin, collagen and fibronectin.

The term “regenerative capacity”, as used herein, refers to conversion of stem cell into dividing progenitor cell and differentiated tissue-specific cell.

The term, “self renewal”, as used herein, refers to proliferation without lineage specification.

The term, “bsp-RGD(15)”, as used herein, refers to the following 15-mer bone sialopeptide sequence: CGGNGEPRGDTYRAY.

The term, “bsp-RGD(15)—FITC”, as used herein, refers to the following bone sialopeptide sequence: CGGNGEPRGDTYRAYK(FITC) GG, wherein FITC refers to.

The term, “bsp-RGE(15)”, as used herein, refers to the following nonsense 5-mer bone sialopeptide sequence: CGGNGEPRGETYRAY.

II. Introduction

The present invention embodies a platform technology consisting of a polymeric material that has properties that resemble an extracellular matrix. This material can be used for tissue formation ex vivo or tissue regeneration in vivo, drug or chemotherapy agent delivery, cell transplantation, and gene therapy. These materials of the invention are of particular use in controlling the destiny of a population of stem cells. Moreover, the materials are of use to deliver stem cells into the body and act as three-dimensional templates to support and promote tissue growth and/or stem cell differentiation. Exemplary materials of the invention are semi-interpenetrating polymer networks (sIPNs) and interpenetrating polymer networks (IPNs). The physical and chemical properties of sIPNs and IPNs (polymers which can contain a significant volume of water) are exploited to mimic the native matrix surrounding mammalian cells (extracellular matrix, ECM), and these networks serve to foster recapitulation of the tissue regeneration process. Exemplary semi-interpenetrating polymer networks (sIPNs) are composed of a cross-linked polymer network with entangled linear polymer chains. sIPNs are of use in a number of applications, including solute delivery and molecular separations. Exemplary interpenetrating polymer networks (IPNs) are composed of two cross-linked polymer networks.

Human embryonic stem cells (hESCs) are being studied as potential source of cells for the treatment for many diseases (e.g. diabetes, Parkinson's, leukemia). The successful integration of hESC into such therapies will hinge upon three critical steps: stem cell expansion in number without differentiating (i.e., self-renewal); differentiation into a specific cell type or collection of cell types; and, promotion of their functional integration into existing tissue. Precisely controlling each of these steps will be essential to maximize hESC's therapeutic efficacy, as well as to minimize potential side effects that can occur when the cells numbers and types are not properly controlled. However, it is difficult to precisely control the behavior of hESCs, since environmental conditions for self-renewal and differentiation are incompletely understood. Currently, hESCs are typically grown on a feeder layer of mouse cells (i.e., irradiated but viable cells) and/or conditioned with media derived from these cells. Thus, current hES cell lines are “contaminated” by foreign, immunogenic oligosaccharide residues acquired from the murine feeder cells and culture medium, and therefore have limited clinical potential. Although newer hES cell lines have been derived on human feeder layers, this system suffers from poor reproducibility and presents limits for large-scale hESC expansion. This invention provides a completely synthetic environment to precisely control hES self-renewal.

II. Compositions of Matter

II.a) IPNs

In a first aspect, the invention provides a network which is an interpenetrating polymer network. The interpenetrating polymer network includes (a) a first cross-linked polymer; and (b) a second cross-linked polymer. Covalently grafted to the first cross-linked polymer and/or the second cross-linked polymer is a ligand which affects the adhesion of the stem cell to the network or the growth or differentiation of a stem cell. Exemplary ligands of use in the invention, such as adhesion peptides, growth factors and differentiation factors, are defined below.

The properties of the cross-linked polymers of the invention can be varied by choice of monomer(s), cross-linking agent and degree of polymer cross-linking. An exemplary variation in the monomer properties is hydrophobicity/hydrophilicity.

In general, providing larger hydrophobic moieties on a cross-linked polymer decreases water swellability. For example, hydrogels made of isopropyl acrylamide are water swellable and possess small hydrophobic moieties (i.e., an isopropyl group). The hydrophobic binding character of these gels is salt dependent. However, when the isopropyl group is replaced by a larger hydrophobic moiety, e.g., an octyl group, the gel loses some of its water swellability.

Exemplary hydrophilic moieties are derived from monomers that include N-methacryloyl-tris(hydroxymethyl)methylamine, hydroxyethyl acrylamide, hydroxypropyl methacrylamide, N-acrylamido-1-deoxysorbitol, hydroxyethylmethacrylate, hydroxypropylacrylate, hydroxyphenylmethacrylate, poly(ethylene glycol)monomethacrylate, poly(ethylene glycol) dimethacrylate, acrylamide, glycerol monomethacrylate, 2-hydroxypropyl acrylate, 4-hydroxybutyl methacrylate, 2-methacryloxyethyl glucoside, poly(ethyleneglycol) monomethyl ether monomethacrylate, vinyl 4-hydroxybutyl ether, and derivatives thereof.

Presently preferred hydrophilic moieties are derived from monomers that include a poly(oxyalkylene) group within their structure. Poly(ethylene glycol)-containing monomers are particularly preferred. PEG of any molecular weight, e.g., 100 Da, 200 Da, 250 Da, 300 Da, 350 Da, 400 Da, 500 Da, 550 Da, 600 Da, 650 Da, 700 Da, 750 Da, 800 Da, 850 Da, 900 Da, 950 Da, 1 kDa, 1500 Da, 2 kDa, 5 kDa, 10 kDa, 15 kDa, 20 kDa, 30 kDa and 40 kDa is of use in the present invention.

Presently preferred hydrophobic moieties are derived from acrylamide monomers in which the amine nitrogen of the amide group is substituted with one or more alkyl residues.

Exemplary hydrophobic moieties are derived from monomers selected from N-isopropylacrylamide, N,N-dimethylacrylamide, N,N-diethyl(meth)acrylamide, N-methyl methacrylamide, N-ethylmethacrylamide, N-propylacrylamide, N-butylacrylamide, N-octyl (meth)acrylamide, N-dodecylmethacrylamide, N-octadecylacrylamide, propyl(meth)acrylate, decyl(meth)acrylate, stearyl(meth)acrylate, octyl-triphenylmethylacrylamide, butyl-triphenylmethylacrylamide, octadedcyl-triphenylmethylacrylamide, phenyl-triphenylmethylacrlamide, benzyl-triphenylmethylacrylamide, and derivatives thereof.

An exemplary cross-linked polymer is a thermoresponsive polymer that changes from a first state to a second when the ambient temperature to which it is exposed is changed. Thus, in an exemplary embodiment, the invention utilizes a thermo-responsive polymer that becomes more rigid, and less flowable, generally more closely resembling an ECM, as it is heated. A preferred polymer changes state, becoming more rigid, within a temperature range that includes mammalian body temperatures, particularly 37° C.

In yet a further exemplary embodiment, the network includes a cross-linked polymer having a subunit derived from a synthetic polymer, peptide, nucleic acid and/or carbohydrate.

In an exemplary embodiment, the cross-linked polymer of the network comprises a subunit derived from N-isopropylacrylamide. In another exemplary embodiment, the cross-linked polymer is N-isopropylacrylamide.

Methods of Making the IPNs

Methods of making IPNs are known in the art. Examples of IPN synthesis are provided in the Examples section.

Cross-linking groups can be used to form the cross-links in either the IPNs or the sIPNs. The following discussion can also apply and to attach the method of attaching the ligand to the network. Thus, the discussion that follows is relevant to both types of cross-linking interactions: ligand cross-linking to the cross-linked or linear polymer; and cross-links within the thermo-responsive polymer.

Both the amount and the identity of the cross-linking agent used in the embodiments of the present invention are variable without limitation. For example, the amount of the cross-linking agent with respect to the polymerizable monomers can vary and it is well within the abilities of one of skill in the art to determine an appropriate amount of cross-linking agent to form an IPN or a sIPN having desired characteristics. In an exemplary embodiment, the cross-linking agent is used in an amount ranging preferably from 0.0001 weight parts to 10 weight parts, more preferably from 0.001 weight parts to 5 weight parts, most preferably from 0.01 weight parts to 2 weight parts, based on 100 parts by weight of either the hydrophobic or hydrophilic monomer.

Exemplary bifunctional compounds which can be used in the present invention include, but are not limited to, bifunctional poly(ethyleneglycols), polyamides, polyethers, polyesters and the like. General approaches for cross-linking two components are known in the literature. See, for example, Lee et al., Biochemistry 28: 1856 (1989); Bhatia et al., Anal. Biochem. 178: 408 (1989); Janda et al., J. Am. Chem. Soc. 112: 8886 (1990) and Bednarski et al., WO 92/18135. In the discussion that follows, the reactive groups are discussed as components of the linear polymer. The focus of the discussion is for clarity of illustration. Those of skill in the art will appreciate that the discussion is relevant to reactive groups on the ligand as well.

In an exemplary strategy for species that contain thiol groups (e.g., proteins or synthetic peptides containing cysteine residues), the —SH groups are grafted to the —COO⁻ groups of, e.g., the p(AAc) chains using the cross-linker N-ε-(maleimidocaproic acid) hydrazide (EMCH; Pierce, Rockford, Ill.). The hydrazide end of EMCH is first reacted with the —COO⁻ groups in the p(AAc) chains using a dehydation agent such as, 1-ethyl-3-(3-dimethylaminopropyl) carbodiimide in the presence of N-hydroxysulfosuccinimide in 2-(N-morpholino)ethanesulfonic acid. The unreacted components are removed via dialysis, the product is lyophylized, and then the maleimide end of EMCH is reacted with the —SH groups of the biomolecule in sodium phosphate buffer (pH 6.6).

Another exemplary strategy involves incorporation of a protected sulfhydryl onto the polymer chain using the heterobifunctional crosslinker SPDP (n-succinimidyl-3-(2-pyridyldithio)propionate and then deprotecting the sulfhydryl for formation of a disulfide bond with another sulfhydryl on the modifying group.

If SPDP detrimentally affects the properties of the linear polymer, there is an array of other crosslinkers such as 2-iminothiolane or N-succinimidyl S-acetylthioacetate (SATA), available for forming disulfide bonds. 2-iminothiolane reacts with primary amines, instantly incorporating an unprotected sulfhydryl onto the amine-containing molecule. SATA also reacts with primary amines, but incorporates a protected sulfhydryl, which is later deacetaylated using hydroxylamine to produce a free sulfhydryl. In each case, the incorporated sulfhydryl is free to react with other sulfhydryls or protected sulfhydryl, like SPDP, forming the required disulfide bond.

The above-described strategies are exemplary, and not limiting, of linkers of use in the invention. Other crosslinkers are available that can be used in different strategies for crosslinking the modifying group to the peptide. For example, TPCH(S-(2-thiopyridyl)-L-cysteine hydrazide and TPMPH ((S-(2-thiopyridyl) mercapto-propionohydrazide) react with aldehydes, thus forming a hydrazone bond between the hydrazide portion of the crosslinker and the periodate generated aldehydes. TPCH and TPMPH introduce a 2-pyridylthione protected sulfhydryl group onto a species, which can be deprotected with DTT and then subsequently used for conjugation, such as forming disulfide bonds between components.

If disulfide bonding is found unsuitable for producing stable networks, other crosslinkers may be used that incorporate more stable bonds between components. The heterobifunctional crosslinkers GMBS (N-gama-malimidobutyryloxy)succinimide) and SMCC (succinimidyl 4-(N-maleimido-methyl)cyclohexane) react with primary amines, thus introducing a maleimide group onto the component. The maleimide group can subsequently react with sulfhydryls on the other component, which can be introduced by previously mentioned crosslinkers, thus forming a stable thioether bond between the components. If steric hindrance between components interferes with either component's activity or the ability of the linear polymer to act as a glycosyltransferase substrate, crosslinkers can be used which introduce long spacer arms between components and include derivatives of some of the previously mentioned crosslinkers (i.e., SPDP). Thus, there is an abundance of suitable crosslinkers, which are useful; each of which is selected depending on the effects it has on optimal peptide conjugate and linear polymer production.

A variety of reagents are used to modify the components of the networks with intramolecular chemical crosslinks (for reviews of crosslinking reagents and crosslinking procedures see: Wold, F., Meth. Enzymol. 25: 623-651, 1972; Weetall, H. H., and Cooney, D. A., In: ENZYMES AS DRUGS. (Holcenberg, and Roberts, eds.) pp. 395-442, Wiley, New York, 1981; Ji, T. H., Meth. Enzymol. 91: 580-609, 1983; Mattson et al., Mol. Biol. Rep. 17: 167-183, 1993, all of which are incorporated herein by reference). Preferred crosslinking reagents are derived from various zero-length, homo-bifunctional, and hetero-bifunctional crosslinking reagents. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. In addition to these chemical reagents, the enzyme transglutaminase (glutamyl-peptide γ-glutamyltransferase; EC 2.3.2.13) may be used as zero-length crosslinking reagent. This enzyme catalyzes acyl transfer reactions at carboxamide groups of protein-bound glutaminyl residues, usually with a primary amino group as substrate. Preferred homo- and hetero-bifunctional reagents contain two identical or two dissimilar sites, respectively, which may be reactive for amino, sulfhydryl, guanidino, indole, or nonspecific groups.

i. Preferred Specific Sites in Crosslinking Reagents

1. Amino-Reactive Groups

In one preferred embodiment, the sites on the cross-linker are amino-reactive groups. Useful non-limiting examples of amino-reactive groups include N-hydroxysuccinimide (NHS) esters, imidoesters, isocyanates, acylhalides, arylazides, p-nitrophenyl esters, aldehydes, and sulfonyl chlorides.

NHS esters react preferentially with the primary (including aromatic) amino groups of a sIPN component. The imidazole groups of histidines are known to compete with primary amines for reaction, but the reaction products are unstable and readily hydrolyzed. The reaction involves the nucleophilic attack of an amine on the acid carboxyl of an NHS ester to form an amide, releasing the N-hydroxysuccinimide. Thus, the positive charge of the original amino group is lost.

Imidoesters are the most specific acylating reagents for reaction with the amine groups of the sIPN components. At a pH between 7 and 10, imidoesters react only with primary amines. Primary amines attack imidates nucleophilically to produce an intermediate that breaks down to amidine at high pH or to a new imidate at low pH. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained.

Isocyanates (and isothiocyanates) react with the primary amines of the sIPN components to form stable bonds. Their reactions with sulfhydryl, imidazole, and tyrosyl groups give relatively unstable products.

Acylazides are also used as amino-specific reagents in which nucleophilic amines of the affinity component attack acidic carboxyl groups under slightly alkaline conditions, e.g. pH 8.5.

Arylhalides such as 1,5-difluoro-2,4-dinitrobenzene react preferentially with the amino groups and tyrosine phenolic groups of sIPN components, but also with sulfhydryl and imidazole groups.

p-Nitrophenyl esters of mono- and dicarboxylic acids are also useful amino-reactive groups. Although the reagent specificity is not very high, α- and ε-amino groups appear to react most rapidly.

Aldehydes such as glutaraldehyde react with primary amines of the linear polymer or components of the cross-linked polymer. Although unstable Schiff bases are formed upon reaction of the amino groups with the aldehydes of the aldehydes, glutaraldehyde is capable of modifying a component of the sIPN with stable crosslinks. At pH 6-8, the pH of typical crosslinking conditions, the cyclic polymers undergo a dehydration to form α-β unsaturated aldehyde polymers. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product.

Aromatic sulfonyl chlorides react with a variety of sites of the sIPN components, but reaction with the amino groups is the most important, resulting in a stable sulfonamide linkage.

2. Sulfhydryl-Reactive Groups

In another preferred embodiment, the sites are sulfhydryl-reactive groups. Useful, non-limiting examples of sulfhydryl-reactive groups include maleimides, alkyl halides, pyridyl disulfides, and thiophthalimides.

Maleimides react preferentially with the sulfhydryl group of the IPN or sIPN components to form stable thioether bonds. They also react at a much slower rate with primary amino groups and the imidazole groups of histidines. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryls via disulfide exchange to give mixed disulfides. As a result, pyridyl disulfides are the most specific sulfhydryl-reactive groups.

Thiophthalimides react with free sulfhydryl groups to form disulfides.

3. Carboxyl-Reactive Residue

In another embodiment, carbodiimides soluble in both water and organic solvent, are used as carboxyl-reactive reagents. These compounds react with free carboxyl groups forming a pseudourea that can then couple to available amines yielding an amide linkage teach how to modify a carboxyl group with carbodiimde (Yamada et al., Biochemistry 20: 4836-4842, 1981).

ii. Preferred Nonspecific Sites in Crosslinking Reagents

In addition to the use of site-specific reactive moieties, the present invention contemplates the use of non-specific reactive groups to link together two components of the IPN or sIPN.

Exemplary non-specific cross-linkers include photoactivatable groups, completely inert in the dark, which are converted to reactive species upon absorption of a photon of appropriate energy. In one preferred embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently preferred. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wave length. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In another preferred embodiment, photoactivatable groups are selected from fluorinated arylazides. The photolysis products of fluorinated arylazides are arylnitrenes, all of which undergo the characteristic reactions of this group, including C—H bond insertion, with high efficiency (Keana et al., J. Org. Chem. 55: 3640-3647, 1990).

In another embodiment, photoactivatable groups are selected from benzophenone residues. Benzophenone reagents generally give higher crosslinking yields than arylazide reagents.

In another embodiment, photoactivatable groups are selected from diazo compounds, which form an electron-deficient carbene upon photolysis. These carbenes undergo a variety of reactions including insertion into C—H bonds, addition to double bonds (including aromatic systems), hydrogen attraction and coordination to nucleophilic centers to give carbon ions.

In still another embodiment, photoactivatable groups are selected from diazopyruvates. For example, the p-nitrophenyl ester of p-nitrophenyl diazopyruvate reacts with aliphatic amines to give diazopyruvic acid amides that undergo ultraviolet photolysis to form aldehydes. The photolyzed diazopyruvate-modified affinity component will react like formaldehyde or glutaraldehyde forming crosslinks.

iii. Homobifunctional Reagents

1. Homobifunctional Crosslinkers Reactive with Primary Amines

Synthesis, properties, and applications of amine-reactive cross-linkers are commercially described in the literature (for reviews of crosslinking procedures and reagents, see above). Many reagents are available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional NHS esters include disuccinimidyl glutarate (DSG), disuccinimidyl suberate (DSS), bis(sulfosuccinimidyl) suberate (BS), disuccinimidyl tartarate (DST), disulfosuccinimidyl tartarate (sulfo-DST), bis-2-(succinimidooxycarbonyloxy)ethylsulfone (BSOCOES), bis-2-(sulfosuccinimidooxycarbonyloxy)ethylsulfone (sulfo-BSOCOES), ethylene glycolbis(succinimidylsuccinate) (EGS), ethylene glycolbis(sulfosuccinimidylsuccinate) (sulfo-EGS), dithiobis(succinimidylpropionate (DSP), and dithiobis(sulfosuccinimidylpropionate(sulfo-DSP). Preferred, non-limiting examples of homobifunctional imidoesters include dimethyl malonimidate (DMM), dimethyl succinimidate (DMSC), dimethyl adipimidate (DMA), dimethyl pimelimidate (DMP), dimethyl suberimidate (DMS), dimethyl-3,3′-oxydipropionimidate (DODP), dimethyl-3,3′-(methylenedioxy)dipropionimidate (DMDP), dimethyl-,3′-(dimethylenedioxy)dipropionimidate (DDDP), dimethyl-3,3′-(tetramethylenedioxy)dipropionimidate (DTDP), and dimethyl-3,3′-dithiobispropionimidate (DTBP).

Preferred, non-limiting examples of homobifunctional isothiocyanates include: p-phenylenediisothiocyanate (DITC), and 4,4′-diisothiocyano-2,2′-disulfonic acid stilbene (DIDS).

Preferred, non-limiting examples of homobifunctional isocyanates include xylene-diisocyanate, toluene-2,4-diisocyanate, toluene-2-isocyanate-4-isothiocyanate, 3-methoxydiphenylmethane-4,4′-diisocyanate, 2,2′-dicarboxy-4,4′-azophenyldiisocyanate, and hexamethylenediisocyanate.

Preferred, non-limiting examples of homobifunctional arylhalides include 1,5-difluoro-2,4-dinitrobenzene (DFDNB), and 4,4′-difluoro-3,3′-dinitrophenyl-sulfone.

Preferred, non-limiting examples of homobifunctional aliphatic aldehyde reagents include glyoxal, malondialdehyde, and glutaraldehyde.

Preferred, non-limiting examples of homobifunctional acylating reagents include nitrophenyl esters of dicarboxylic acids.

Preferred, non-limiting examples of homobifunctional aromatic sulfonyl chlorides include phenol-2,4-disulfonyl chloride, and α-naphthol-2,4-disulfonyl chloride.

Preferred, non-limiting examples of additional amino-reactive homobifunctional reagents include erythritolbiscarbonate which reacts with amines to give biscarbamates.

2. Homobifunctional Crosslinkers Reactive with Free Sulfhydryl Groups

Synthesis, properties, and applications of such reagents are described in the literature (for reviews of crosslinking procedures and reagents, see above). Many of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional maleimides include bismaleimidohexane (BMH), N,N′-(1,3-phenylene) bismaleimide, N,N′-(1,2-phenylene)bismaleimide, azophenyldimaleimide, and bis(N-maleimidomethyl)ether.

Preferred, non-limiting examples of homobifunctional pyridyl disulfides include 1,4-di-3′-(2′-pyridyldithio)propionamidobutane (DPDPB).

Preferred, non-limiting examples of homobifunctional alkyl halides include 2,2′-dicarboxy-4,4′-diiodoacetamidoazobenzene, α,α′-diiodo-p-xylenesulfonic acid, α,α′-dibromo-p-xylenesulfonic acid, N,N′-bis(b-bromoethyl)benzylamine, N,N′-di(bromoacetyl)phenylthydrazine, and 1,2-di(bromoacetyl)amino-3-phenylpropane.

3. Homobifunctional Photoactivatable Crosslinkers

Synthesis, properties, and applications of such reagents are described in the literature (for reviews of crosslinking procedures and reagents, see above). Some of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional photoactivatable crosslinker include bis-β-(4-azidosalicylamido)ethyldisulfide (BASED), di-N-(2-nitro-4-azidophenyl)cystamine-S,S-dioxide (DNCO), and 4,4′-dithiobisphenylazide.

iv. HeteroBifunctional Reagents

1. Amino-Reactive HeteroBifunctional Reagents with a Pyridyl Disulfide Moiety

Synthesis, properties, and applications of such reagents are described in the literature (for reviews of crosslinking procedures and reagents, see above). Many of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of hetero-bifunctional reagents with a pyridyl disulfide moiety and an amino-reactive NHS ester include N-succinimidyl-3-(2-pyridyldithio)propionate (SPDP), succinimidyl 6-3-(2-pyridyldithio)propionamidohexanoate (LC-SPDP), sulfosuccinimidyl 6-3-(2-pyridyldithio)propionamidohexanoate (sulfo-LCSPDP), 4-succinimidyloxycarbonyl-α-methyl-α-(2-pyridyldithio)toluene (SMPT), and sulfosuccinimidyl 6-α-methyl-α-(2-pyridyldithio)toluamidohexanoate (sulfo-LC-SMPT).

2. Amino-Reactive HeteroBifunctional Reagents with a Maleimide Moiety

Synthesis, properties, and applications of such reagents are described in the literature. Preferred, non-limiting examples of hetero-bifunctional reagents with a maleimide moiety and an amino-reactive NHS ester include succinimidyl maleimidylacetate (AMAS), succinimidyl 3-maleimidylpropionate (BMPS), N-γ-maleimidobutyryloxysuccinimide ester (GMBS)N-γ-maleimidobutyryloxysulfosuccinimide ester (sulfo-GMBS) succinimidyl 6-maleimidylhexanoate (EMCS), succinimidyl 3-maleimidylbenzoate (SMB), m-maleimidobenzoyl-N-hydroxysuccinimide ester (MBS), m-maleimidobenzoyl-N-hydroxysulfosuccinimide ester (sulfo-MBS), succinimidyl 4-(N-maleimidomethyl)cyclohexane-1-carboxylate (SMCC), sulfosuccinimidyl 4-(N-maleimidomethyl)cyclohexane-1-carboxylate (sulfo-SMCC), succinimidyl 4-(p-maleimidophenyl)butyrate (SMPB), and sulfosuccinimidyl 4-(p-maleimidophenyl)butyrate (sulfo-SMPB).

3. Amino-Reactive HeteroBifunctional Reagents with an Alkyl Halide Moiety

Synthesis, properties, and applications of such reagents are described in the literature. Preferred, non-limiting examples of hetero-bifunctional reagents with an alkyl halide moiety and an amino-reactive NHS ester include N-succinimidyl-(4-iodoacetyl)aminobenzoate (SIAB), sulfosuccinimidyl-(4-iodoacetyl)aminobenzoate (sulfo-SIAB), succinimidyl-6-(iodoacetyl)aminohexanoate (SIAX), succinimidyl-6-(6-((iodoacetyl)-amino)hexanoylamino)hexanoate (SIAXX), succinimidyl-6-(((4-(iodoacetyl)-amino)methyl)-cyclohexane-1-carbonyl)aminohexanoate (SIACX), and succinimidyl-4((iodoacetyl)-amino)methylcyclohexane-1-carboxylate (SIAC).

A preferred example of a hetero-bifunctional reagent with an amino-reactive NHS ester and an alkyl dihalide moiety is N-hydroxysuccinimidyl 2,3-dibromopropionate (SDBP). SDBP introduces intramolecular crosslinks to the affinity component by conjugating its amino groups. The reactivity of the dibromopropionyl moiety for primary amino groups is defined by the reaction temperature (McKenzie et al., Protein Chem. 7: 581-592 (1988)).

Preferred, non-limiting examples of hetero-bifunctional reagents with an alkyl halide moiety and an amino-reactive p-nitrophenyl ester moiety include p-nitrophenyl iodoacetate (NPIA).

Other cross-linking agents are known to those of skill in the art (see, for example, Pomato et al., U.S. Pat. No. 5,965,106. It is within the abilities of one of skill in the art to choose an appropriate cross-linking agent for a particular application.

Purification of the Networks of the Invention

The products produced (either IPNs or sIPNs) by the processes described herein can be used without purification. However, it is usually preferred to recover the product. Standard, well-known techniques for recovery of polymers such as thin or thick layer chromatography, column chromatography, ion exchange chromatography, gel permeation chromatography or membrane filtration can be used. It is preferred to use membrane filtration, more preferably utilizing a nanofiltration or reverse osmotic membrane, or one or more column chromatographic techniques for the recovery as is discussed hereinafter and in the literature cited herein. For instance, membrane filtration can be used to remove unreacted or incompletely reacted monomers and oligomers. Nanofiltration or reverse osmosis can be used to remove salts and/or purify the products. Nanofilter membranes are a class of reverse osmosis membranes that pass monovalent salts but retain polyvalent salts and uncharged solutes larger than about 100 to about 2,000 Daltons, depending upon the membrane used. Thus, in a typical application, IPNs or sIPNs prepared by the methods of the present invention will be retained in the membrane and contaminating salts will pass through.

If the IPN or sIPN results in the formation of a solid, the particulate material is removed, for example, by centrifugation or ultrafiltration.

Other methods of purification of IPNs or sIPNs of the invention that are derivatized with a ligand include, e.g., immunoaffinity chromatography, ion-exchange column fractionation (e.g., on diethylaminoethyl (DEAE) or networks containing carboxymethyl or sulfopropyl groups), chromatography on Blue-Sepharose, CM Blue-Sepharose, MONO-Q, MONO-S, lentil lectin-Sepharose, WGA-Sepharose, Con A-Sepharose, Ether Toyopearl, Butyl Toyopearl, Phenyl Toyopearl, or protein A Sepharose, SDS-PAGE chromatography, silica chromatography, chromatofocusing, reverse phase HPLC (e.g., silica gel with appended aliphatic groups), gel filtration using, e.g., Sephadex molecular sieve or size-exclusion chromatography, chromatography on columns that selectively bind the polypeptide, and ethanol or ammonium sulfate precipitation.

A protease inhibitor, e.g., methylsulfonylfluoride (PMSF) may be included in any of the foregoing steps to inhibit proteolysis and antibiotics may be included to prevent the growth of adventitious contaminants.

Finally, one or more RP-HPLC steps employing hydrophobic RP-HPLC media, e.g., silica gel having pendant methyl or other aliphatic groups, may be employed to further purify a polypeptide variant composition. Some or all of the foregoing purification steps, in various combinations, can also be employed to provide a homogeneous modified glycoprotein.

II.b) sIPNs

In a second aspect, the invention provides a network which is a semi-interpenetrating polymer network. The semi-interpenetrating polymer network includes (a) a cross-linked polymer; and (b) a linear polymer entangled within said cross-linked polymer. Covalently grafted to the cross-linked polymer and/or the linear polymer is a ligand which affects the adhesion of the stem cell to the network or the growth or differentiation of a stem cell. Exemplary ligands of use in the invention, such as adhesion peptides, growth factors and differentiation factors, are defined below.

Cross-linking polymers of use in the sIPN are described and discussed in the IPN section. All of the cross-linked polymers discussed herein can be employed in the sIPNs of the invention.

Similar to the cross-linked polymer, properties (e.g., the hydrophobicity/hydrophilicity) of the linear polymer can be varied. Moreover, characteristics of the polymer such as length and number and identity of reactive functional groups can be varied as desired for a particular application.

Useful linear polymer chains include any long-chain polymer that contains a functional group (e.g., —NH₂, —COO⁻, —SH, etc.) that is amenable to modification with biomolecules. Examples of such linear polymers are hyaluronic acid (HA), poly(methacrylic acid), poly(ethylene glycol) (EG), or poly(lysine). The linear polymer chain can also be a copolymer, e.g. p(AAc-co-EG), or a terpolymer. The only requirement for the linear chain is that is amenable to either grafting biological molecules or particles, e.g., for gene therapy and does not interfere with the phase change properties of the cross-linked network.

Another exemplary class of linear polymers is electrically-responsive polymers for fostering growth of electrically-responsive cells such as cardiac myocytes or neurons. In addition to p(AAc), linear chains of poly(methacrylic acid), poly(dimethylaminopropylacrylamide), poly(2-acrylamido-2-methylpropane sulphonic acid), HA, copolymers of these polymers, and other electro-responsive linear polymers that change their shape under an electric field or potential can be incorporated into the sIPN. These chains can be additionally functionalized with biomolecules to make an electrically and bioactive hydrogel capable of stimulating cell growth and alignment. The cellular alignment is caused by the templating of the cells on the aligned electrically active linear polymer chains.

Methods of Making the sIPNs

Methods of making sIPNs are known in the art. Examples of sIPN synthesis are provided in the Examples section.

II.c) Ligands

The networks of the invention also include a ligand, e.g., a biomolecule such as a functional protein, enzyme, antigen, antibody, peptide, nucleic acid (e.g., single nucleotides or nucleosides, oligonucleotides, polynucleotides and single- and higher-stranded nucleic acids), lectin, receptor, saccharide, ganglioside, cerebroside or a combination thereof.

Biomolecules useful in practicing the present invention can be derived from any source. The biomolecules can be isolated from natural sources or they can be produced by synthetic methods. Peptides can be natural peptides or mutated peptides. Mutations can be effected by chemical mutagenesis, site-directed mutagenesis or other means of inducing mutations known to those of skill in the art. Peptides and proteins useful in practicing the instant invention include, for example, enzymes, antigens, antibodies and receptors. Antibodies can be either polyclonal or monoclonal.

Biomolecules of use in the compositions of the present invention include natural and modified biomolecules and therapeutic moieties. The discussion that follows focuses on the use of a peptide as an exemplary biomolecule. The focus is for clarity of illustration only. It will be apparent to those of skill in the art that substantially any biomolecule can be incorporated into the compositions of the invention.

In an exemplary embodiment, the ligand promotes the adhesion, growth or differentiation of a stem cell. Examples of these stem cells include embryonic stem cells, adult marrow stem cells, adult neural stem cells, cord blood stem cells, adult skin stem cells, adult liver stem cells, adult olfactory stem cells, adult adipose-derived stem cells, adult hair follicle stem cells, adult skeletal muscle stem cells, and adult myogenic muscle stem cells.

Exemplary peptides that can be utilized in forming the compositions of the invention are set forth in Table 1.

TABLE 1

Hormones and Growth Factors
G-CSF
GM-CSF
TPO
EPO
EPO variants
alpha-TNF
Leptin
Hedgehogs
Fibroblast Growth Factors
Wnt
Activin
Delta/Notch
Bone Morphogenetic Proteins
TGF-β
Enzymes and Inhibitors
t-PA
t-PA variants
Urokinase
Factors VII, VIII, IX, X
DNase
Glucocerebrosidase
Hirudin
α1 antitrypsin
Antithrombin III
Cytokines and Chimeric Cytokines
Interleukin-1 (IL-1), 1B, 2, 3, 4, 6 and 11
Interferon-alpha (IFN-alpha)
IFN-alpha-2b
IFN-beta
IFN-gamma
Chimeric diptheria toxin-IL-2
Receptors and Chimeric Receptors
CD4
Tumor Necrosis Factor (TNF) receptor
Alpha-CD20
MAb-CD20
MAb-alpha-CD3
MAb-TNF receptor
MAb-CD4
PSGL-1
MAb-PSGL-1
Complement
GlyCAM or its chimera
N-CAM or its chimera
Monoclonal Antibodies (Immunoglobulins)
MAb-anti-RSV
MAb-anti-IL-2 receptor
MAb-anti-CEA
MAb-anti-platelet IIb/IIIa receptor
MAb-anti-EGF
MAb-anti-Her-2 receptor
Cells
Red blood cells
White blood cells (e.g., T cells, B cells,
dendritic cells, macrophages, NK cells,
neutrophils, monocytes and the like
Stem cells

Other exemplary peptides useful in the composition of the invention include members of the immunoglobulin family (e.g., antibodies, MHC molecules, T cell receptors, and the like), intercellular receptors (e.g., integrins, receptors for hormones or growth factors and the like) lectins, and cytokines (e.g., interleukins). Additional examples include tissue-type plasminogen activator (t-PA), renin, clotting factors such as factor VIII and factor IX, bombesin, thrombin, hematopoietic growth factor, colony stimulating factors, viral antigens, complement proteins, α1-antitrypsin, erythropoietin, P-selectin glycopeptide ligand-1 (PSGL-1), granulocyte-macrophage colony stimulating factor, anti-thrombin III, interleukins, interferons, proteins A and C, fibrinogen, herceptin, leptin, glycosidases, among many others. This list of polypeptides is exemplary, not exclusive. The network of the invention can also include a chimeric protein, including, but not limited to, chimeric proteins that include a moiety derived from an immunoglobulin, such as IgG.

Other biomolecules that can be grafted to a network of the invention, include Nestin, Vimentin, Prominin/CD133, Sonic hedgehog and other hedgehog ligands, Wnt ligands, Neurocan/tenascin C, Nurr 1, Pax-6, Sox-2, Musashi-1, NG2/CSPG-4, Neuro D3, Neurogenin 1, and fragments and subsequences of these molecules. Growth factors are also of use in the materials and methods of the invention, e.g., CNTF, BDNF, and GDNF.

Other exemplary biomolecules include Beta tubulin III, MAP2, Neuron specific enolase, NCAM, CD24, HAS, Synapsin I, Synaptophysin, CAMK Iia, Tyrosine hydroxylase, Glutamate transporter, Glutamate receptor, Choline rececptor, nicotinic A2, EphB2, GABA-A receptor, Serotonin (5HT-3) receptor, Choline acetyltransferase and fragments and subsequences thereof. These biomolecules can be particularly important when the stem cell of interest is a neuronal stem cell.

When the cells are astrocytes or progenitors thereof exemplary biomolecules of use in the materials and methods of the invention include GFAP, GAD65, S100 and fragments and subsequences thereof.

When the cells are oligodendrocytes or progenitors thereof, exemplary biomolecules of use in the materials and methods of the invention include Olig1, Plp/DM20, Myelin basic protein, and fragments and subsequences thereof.

Certain disease related biomolecules of use in the invention include, e.g., Presenilin-1, Beta APP, Bcl-2, Huntington's disease protein, and fragments and subsequences thereof.

The invention also provides networks in which the biomolecule is a member selected from GAPDH, Beta actin, Lamin A, Hat1, Hat5, and YBBR, and fragments and subsequences thereof.

In another exemplary embodiment, the biomolecule is a peptide that promotes adhesion of the stem cell to the network. An example is a peptide that contains the arginine-glycine-aspartate (RGD) motif. The RGD tripeptide motif is found in proteins of the extracellular matrix. Integrins link the intracellular cytoskeleton of cells with the extracellular matrix by recognizing peptides that include the RGD motif. RGD peptides interact with the integrin receptor sites, which can initiate cell-signaling processes and influence many different cellular processes (Kantlehner et al., Angew. Chem. Int. Ed. 38: 560 (1999)).

The covalent grafting of RGD peptides to the network provides a novel material that controls cell adhesion to itself and, hence, to other materials to which it is attached. Accordingly, the present invention provides a sIPN that includes a peptide having the RGD motif.

Frequently, active RGD peptides are head-to-tail cyclic pentapeptides. In an exemplary embodiment, the network of the invention includes a ligand which is a cyclic pentapetpide. An exemplary bicyclic RGD peptide, H-Glu[cyclo (Arg-Gly-Asp-D-Phe-Lys)]₂, was recently reported by Janssen et al. to possess high affinity αvβ3 integrin binding (IC50=0.9 nM) with low affinity for αvβ5 and αIIBβ3 integrin (IC50=10 nM) (Janssen et al., Cancer Research 62: 6146 (2002)). In another exemplary embodiment, the peptide is cyclo (Arg-Gly-Asp-D-Phe-Lys).

In another exemplary embodiment, the invention provides a network to stimulate bone formation incorporating the adhesion peptides bsp-RGD(15) [(acetyl)-CGGNGEPRGDTYRAY-NH₂] (-RGD-) and (acetyl)-CGGFHRRIKA-NH₂ (-FHRRIKA-), selected from the cell-binding and heparin-binding domains of bone sialoprotein (BSP), to accelerate proliferation of stem cells in contact with the peptide modified p(NIPAAm-co-AAc) hydrogels.

The peptides of use as ligands in the networks of the invention can also include amino acid residues upon which an array of conjugation reactions can be practiced. For example, a peptide, cyclo(Arg-Gly-Asp-D-Tyr-Lys) incorporates a tyrosine into this active motif for iodination and for glycosylation (Haubner et al., J. Nucl. Med. 42: 326-36 (2001)).

The biomolecule of the invention can be grafted to a network either directly or through a crosslinking agent.

Both naturally derived and synthetic peptides and nucleic acids are of use as ligands in conjunction with the present invention; these molecules can be grafted to a component of the network by any available reactive group. For example, peptides can be grafted through a reactive amine, carboxyl, sulfhydryl, or hydroxyl group. The reactive group can reside at a peptide terminus or at a site internal to the peptide chain. Nucleic acids can be grafted through a reactive group on a base (e.g., exocyclic amine) or an available hydroxyl group on a sugar moiety (e.g., 3′- or 5′-hydroxyl). The peptide and nucleic acid chains can be further derivatized at one or more sites to allow for the attachment of appropriate reactive groups onto the chain. See, Chrisey et al. Nucleic Acids Res. 24: 3031-3039 (1996).

In a further preferred embodiment, the network includes a ligand which is a targeting species that is selected to direct the network of the invention to a specific tissue. Exemplary species of use for targeting applications include signaling peptides, peptides which bind to cell-surface receptors, antibodies and hormones.

The materials of the invention also allow for variation in peptide structure in order to optimize a property of the bound cell, e.g., binding to the material, proliferation, differentiation, etc.

Moreover, the density of the ligand on the network of the invention can be varied. For example, peptide densities from as low as about 0.01 pM/cm² to as high as about 100 pM/cm² are of use in the present invention.

Methods of Conjugating Ligands to a Network of the Invention

Methods of conjugating ligand to networks are well known to those of skill in the art. See, for example Hermanson, BIOCONJUGATE TECHNIQUES, Academic Press, San Diego, 1996; and Dunn et al., Eds. POLYMERIC DRUGS AND DRUG DELIVERY SYSTEMS, ACS Symposium Series Vol. 469, American Chemical Society, Washington, D.C. 1991.

The ligand is grafted to either a cross-linked polymer or a linear polymer either directly or through a cross-linking agent. Either of these modes of attachment can be engineered to produce a linkage that is either stable under biologically relevant conditions, or which is cleaved under selected conditions, releasing the ligand from the network.

In general, the polymers of the networks (either cross-linked or linear) and the ligand are linked together through the use of reactive groups, which are typically transformed by the linking process into a new organic functional group or unreactive species. The reactive functional group(s), is located at any position of the biomolecule and the linear polymer that is convenient. Reactive groups and classes of reactions useful in practicing the present invention are generally those that are well known in the art of bioconjugate chemistry. Currently favored classes of reactions available with reactive species are those, which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in numerous texts and literature references, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Methods and chemistry for activating polymers, as well as methods for conjugating ligands onto polymers, are described in the literature. See, R. F. Taylor, (1991), PROTEIN IMMOBILISATION. FUNDAMENTALS AND APPLICATIONS, Marcel Dekker, N.Y.; S. S. Wong, (1992), CHEMISTRY OF PROTEIN CONJUGATION AND CROSSLINKING, CRC Press, Boca Raton; G. T. Hermanson et al., (1993), IMMOBILIZED AFFINITY LIGAND TECHNIQUES, Academic Press, N.Y.; Dunn, R. L., et al., Eds. POLYMERIC DRUGS AND DRUG DELIVERY SYSTEMS, ACS Symposium Series Vol. 469, American Chemical Society, Washington, D.C. 1991).

Several reviews and monographs on the functionalization and conjugation of PEG are available. See, for example, Harris, Macronol. Chem. Phys. C25: 325-373 (1985); Scouten, Methods in Enzymology 135: 30-65 (1987); Wong et al., Enzyme Microb. Technol. 14: 866-874 (1992); Delgado et al., Critical Reviews in Therapeutic Drug Carrier Systems 9: 249-304 (1992); and Zalipsky, Bioconjugate Chem. 6: 150-165 (1995).

Methods for activation of polymers can also be found in WO 94/17039, U.S. Pat. No. 5,324,844, WO 94/18247, WO 94/04193, U.S. Pat. No. 5,219,564, U.S. Pat. No. 5,122,614, WO 90/13540, U.S. Pat. No. 5,281,698, and more WO 93/15189, and for conjugation between activated polymers and peptides, e.g. Coagulation Factor VIII (WO 94/15625), haemoglobin (WO 94/09027), oxygen carrying molecule (U.S. Pat. No. 4,412,989), ribonuclease and superoxide dismutase (Veronese at al., App. Biochem. Biotech. 11: 141-45 (1985)).

Useful reactive functional groups pendent from a cross-linked polymer, linear polymer or ligand include, but are not limited to:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups, which can be converted to, e.g., esters, ethers, aldehydes, etc.
(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the functional group of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be, for example, converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc; and
(j) epoxides, which can react with, for example, amines and hydroxyl compounds.

The reactive functional groups can be chosen such that they do not participate in, or interfere with, the reactions necessary to assemble the IPN, sIPN or their components. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

II.d) Degradable Cross-Links

In another aspect, the IPN or sIPN can comprise a degradable cross-linker. This cross-linker can be used to attach the ligand to the cross-linked polymer or the linear polymer. The cross-linker can also be used as a component of the cross-linked polymer. the cross-linker can be cleaved to dissociate the cross-linked species.

Many cleaveable groups are known in the art. See, for example, Jung et al., Biochem. Biophys. Acta 761: 152-162 (1983); Joshi et al., J. Biol. Chem. 265: 14518-14525 (1990); Zarling et al., J. Immunol. 124: 913-920 (1980); Bouizar et al., Eur. J. Biochem. 155: 141-147 (1986); Park et al., J. Biol. Chem. 261: 205-210 (1986); Browning et al., J. Immunol. 143: 1859-1867 (1989). Moreover a broad range of cleavable, bifunctional (both homo- and hetero-bifunctional) linker groups are commercially available from suppliers such as Pierce.

Exemplary cleaveable moieties can be cleaved using light, heat or reagents such as thiols, hydroxylamine, bases, periodate and the like. Moreover, certain preferred groups are cleaved in vivo in response to their being endocytized (e.g., cis-aconityl; see, Shen et al., Biochem. Biophys. Res. Commun. 102: 1048 (1991)). Preferred cleaveable groups comprise a cleaveable moiety which is a member selected from the group consisting of disulfide, ester, imide, carbonate, nitrobenzyl, phenacyl and benzoin groups.

In another exemplary embodiment, the crosslinkers are degradable via hydrolysis. Examples of such cross-linkers include poly(glycolide) [poly(glycolic acid)], poly(lactide) (pL) [poly(lactic acid], poly(ε-caprolactone) (pEC), other α-hydroxy acid esters, and copolymers of these materials with pEG [e.g., random, block].

In yet another exemplary embodiment, the IPNs and sIPNs of the invention are used in the context of the natural process of proteolytic remodeling of the extracellular matrix, which is essential in tissue morphogenesis during fetal development, inflammation, arthritis, cancer, and wound healing and tissue regeneration (Massova et al., FASEB Journal, 12:1075-1095 (1998); Johansson et al., Developmental Dynamics, 208:387-397 (1997)). To make the networks degradable oligopeptide crosslinkers that are specifically cleaved by the matrix metalloproteinase (MMP) family are incorporated into the IPNs and sIPNs. MMPs are a structurally and functionally related family of zinc-dependent endopeptidases that cleave either one or several ECM proteins (Massova et al., FASEB Journal, 12:1075-1095 (1998)). Recently, West and Hubbell (West et al., Macromolecules, 32:241-244 (1999)) developed a new class of telechelic biodegradable block copolymers that when synthesized into a crosslinked hydrogel were specifically degraded by either plasmin or crude collagenase. Thus, the feasibility of protease degradation of oligopeptide crosslinked hydrogels has been demonstrated in vitro (West et al., Macromolecules, 32:241-244 (1999)).

An exemplary embodiment of the invention is an IPN or sIPN which incorporates peptide crosslinkers that are cleaved by collagenase-3 (MMP-13). Since MMP-13 has primary, secondary, and tertiary cleavage sites for type II collagen, all with different enzyme-substrate affinity (K_{M}) and maximal catalytic rate when substrate is saturating (k_{cat}), (Mitchell et al., Journal of Clinical Investigation, 97:761-768 (1996)) then theoretically the degradation rate of the hydrogel could be tailored by selecting peptides with the appropriate cleavage site.

In an exemplary embodiment, the IPN or sIPN of the invention includes a peptide crosslinker (see Example 8 for a discussion specifically involving sIPNs) as a component. The degradation rates of the IPNs and sIPNs with peptide crosslinkers can be altered by synthesizing the network with mixed crosslinkers with different cleavage sites for MMP-13, e.g. primary versus tertiary sites, by changing the crosslinker density, and by changing substrate length or amino acids flanking the cleavage site (West et al., Macromolecules, 32:241-244 (1999); (Netzel-Arnett et al., Journal of Biological Chemistry, 266:6747-6755 (1991)). The aforementioned modifications to the networks alter the degradation rates by changing k_{cat}/K_{M}, an index of substrate specificity.

Peptide crosslinkers can be synthesized on a commercial peptide synthesizer, purified, and verified to be >97% pure by HPLC and mass spectroscopy. The peptides are synthesized using standard methods with side group protection. Protection of the amine groups is critical since it is important for the docking of the MMP-13 to the peptide substrate (Mitchell et al., Journal of Clinical Investigation, 97:761-768 (1996)). To acrylate the peptides, while still on the resin, the Fmoc protection group from the N terminus is cleaved with 20% piperidine in dimethylformamide (DMF) and the free amine is acrylated by reacting acrylic acid with 1-ethyl-3-(3-dimethylaminopropyl) carbodiimide (EDC, Pierce, Rockford, Ill., USA) with the NH₂ in a similar manner to that described previously by Bearinger et al. (Bearinger et al., Journal of Biomaterials Science, Polymer Edition, 9:629-52 (1998)). Briefly, the carboxylic acid on the acrylic acid is linked to the N terminal amine by inducing a carbodiimide reaction utilizing 0.400 mg/ml EDC and 1.100 mg/ml N-Hydroxysulfosuccinimide (Sulfo-NHS, Pierce) in [2-(N-Morpholino)ethanesulfonic acid, 0.100 M, in 0.5 M NaCl conjugation buffer (MES, Pierce) at a pH of 6.0. Although this pH is low, it is not nearly low enough to cleave the peptide off the resin or remove side chain protection. The reaction proceeds for 1 h, and then the resin is rinsed with 10% TFA to cleave the peptide from the resin with side group protection intact. The carboxyl termini is acrylated in solution by reacting the —COOH with ethylenediamine with EDC (similar conditions as above) to generate a free amine and then following the reaction scheme outlined above for coupling acrylic acid with the —NH₂.

To synthesize the degradable network, the synthetic route and conditions for polymerization for a non-degradable network is used, replacing the non-degradable crosslinker with the peptide crosslinkers. The side chain protection groups on the cross-linkers are deprotected, e.g., with 90% TFA prior to synthesis. Degradable networks synthesized as described above can be used in a similar manner to the non-degradable networks; however, the scaffold will be temporary based on the enzymatic cleavage of the cross-links.

II.e) Stem Cells

In another aspect, stem cells can be incorporated into the networks of the invention. In an exemplary embodiment, the stem cells are from a mammalian species. Included are stem cells from humans; as well as non-human primates, domestic animals, livestock, and other non-human mammals. In an exemplary embodiment, embryonic stem cells, adult marrow stem cells, adult neural stem cells, cord blood stem cells, adult skin stem cells, adult liver stem cells, adult olfactory stem cells, adult adipose-derived stem cells, adult hair follicle stem cells, adult skeletal muscle stem cells, and/or adult myogenic muscle stem cells are incorporated into the networks. Amongst the stem cells suitable for use in this invention are primate pluripotent stem (pPS) cells derived from tissue formed after gestation, such as a blastocyst, or fetal or embryonic tissue taken any time during gestation. Other non-limiting examples include primary cultures or established lines of embryonic stem cells.

In an exemplary embodiment, the invention provides a stem cell that is immobilized on (bound to) a network of the invention. In another embodiment, the invention provides a population of stem cells that are immobilized on a network of the invention. In still a further exemplary embodiment, the invention provides a population of undifferentiated stem cells mixed with a population of differentiated cells, wherein the members of each population is bound to a sIPN of the invention.

Sources of Stem Cells

This invention can be practiced using stem cells of various types, which may be obtained from sources such as the following non-limiting examples. U.S. Pat. No. 5,851,832 reports multipotent neural stem cells obtained from brain tissue. U.S. Pat. No. 5,766,948 reports producing neuroblasts from newborn cerebral hemispheres. U.S. Pat. Nos. 5,654,183 and 5,849,553 report the use of mammalian neural crest stem cells. U.S. Pat. No. 6,040,180 reports in vitro generation of differentiated neurons from cultures of mammalian multipotential CNS stem cells. WO 98/50526 and WO 99/01159 report generation and isolation of neuroepithelial stem cells, oligodendrocyte-astrocyte precursors, and lineage-restricted neuronal precursors. U.S. Pat. No. 5,968,829 reports neural stem cells obtained from embryonic forebrain and cultured with a medium comprising glucose, transferrin, insulin, selenium, progesterone, and several other growth factors.

When the stem cells are derived from the liver, primary liver cell cultures can be obtained from human biopsy or surgically excised tissue by perfusion with an appropriate combination of collagenase and hyaluronidase. Alternatively, EP 0 953 633 reports isolating liver cells by preparing minced human liver tissue, resuspending concentrated tissue cells in a growth medium and expanding the cells in culture. The growth medium comprises glucose, insulin, transferrin, T₃, FCS, and various tissue extracts that allow the hepatocytes to grow without malignant transformation. The cells in the liver are thought to contain specialized cells including liver parenchymal cells, Kupffer cells, sinusoidal endothelium, and bile duct epithelium, and also precursor cells (referred to as “hepatoblasts” or “oval cells”) that have the capacity to differentiate into both mature hepatocytes or biliary epithelial cells (Rogler, Am. J. Pathol. 150: 591 (1997); Alison, Current Opin. Cell Biol. 10: 710 (1998); Lazaro et al., Cancer Res. 58: 514 (1998).

U.S. Pat. No. 5,192,553 reports methods for isolating human neonatal or fetal hematopoietic stem or progenitor cells. U.S. Pat. No. 5,716,827 reports human hematopoietic cells that are Thy-1 positive progenitors, and appropriate growth media to regenerate them in vitro. U.S. Pat. No. 5,635,387 reports a method and device for culturing human hematopoietic cells and their precursors. U.S. Pat. No. 6,015,554 describes a method of reconstituting human lymphoid and dendritic cells.

U.S. Pat. No. 5,486,359 reports homogeneous populations of human mesenchymal stem cells that can differentiate into cells of more than one connective tissue type, such as bone, cartilage, tendon, ligament, and dermis. They are obtained from bone marrow or periosteum. Also reported are culture conditions used to expand mesenchymal stem cells. WO 99/01145 reports human mesenchymal stem cells isolated from peripheral blood of individuals treated with growth factors such as G-CSF or GM-CSF. WO 00/53795 reports adipose-derived stem cells and lattices, substantially free of adipocytes and red cells. These cells reportedly can be expanded and cultured to produce hormones and conditioned culture media.

Thomson et al. Science, 282: 1145 (1998) reports the isolation and culturing of human embryonic stem cells.

Assays for Stem Cell Phenotype

Methods for the characterization, validation and quantification of the phenotype of stem cells cultured on a material of the invention are of use in the present invention. The methods are of use for, inter alia, determining whether cells adhering to a material of the invention are proliferating and whether the population or a subset thereof has undergone differentiation.

Representative assays include, but are not limited to measuring cell number, and immunostaining of the cells to determine their phenotype. Immunostaining provides useful information regarding progenitor multipotency. Exemplary immunostaining procedures of use in stem cells and their progeny utilize antibodies directed to both undifferentiated and differentiated cells, e.g., anti-nestin, anti-β-tubulin III, anti-GFAP, and anti-O4, and antibodies against OCT-4 and SSEA-4, for neural stem cell cultures. The primary antibodies can be stained with detectably labeled secondary antibodies. The stained cells can be classified using fluorescence microscopy or fluorescence flow cytometry. The fraction of cells in each undifferentiated or differentiated state can be counted.

Other methods rely on the lineage specific promoter driving the expression of a reporter gene, e.g., Green Fluorescent Protein.

In another embodiment, the invention relies on the use of quantitative reverse transcriptase PCR (qRT-PCR). This method is of use to detect lineage specific markers during progenitor differentiation. The method is of use in high throughput analyses. Moreover, DNA microarray analysis on stem cell populations grown within various networks can help refine and identify which lineage specific markers are most relevant during differentiation and proliferation.

It is well within the abilities of one of skill in the art to determine an appropriate assay to determine the phenotype of a population of cells bound to a network of the invention.

II.f) Tuning the IPNs and sIPNs

IPNs and sIPNs of the invention can possess a variety of different mechanical and biochemical properties. Depending on the temperature, identity and concentration of the network components, mechanical properties such as the shear modulus (G) Young's modulus (E), complex shear modulus, complex Young's modulus and loss angle can be manipulated. Depending on the identity and concentration of the network components, ligand density, ligand type and method of ligand attachment, biochemical properties such as non-stem cell biological interactions (fouling) stem cell growth, differentiation, and rates of growth and differentiation, can be manipulated.

In an exemplary embodiment, the ligand has a density in the network of from 0.1 pmol/cm² to 20 pmol/cm². In an exemplary embodiment, the density is from 0.1 to 0.5. In an exemplary embodiment, the density is from 0.1 to 1. In an exemplary embodiment, the density is from 1 to 8. In an exemplary embodiment, the density is from 5 to 20. In an exemplary embodiment, the density is from 5 to 14. In an exemplary embodiment, the density is from 0.5 to 9.

In an exemplary embodiment, the ligand has a density in the network of from 50 μM to 500 μM. In an exemplary embodiment, the ligand has a density in the network of from 75 μM to 400 μM. In an exemplary embodiment, the ligand has a density in the network of from 100 μM to 240 μM. In an exemplary embodiment, the ligand has a density in the network of from 350 μM to 500 μM. In an exemplary embodiment, the ligand has a density in the network of from 175 μM to 375 μM. In an exemplary embodiment, the ligand has a density in the network of from 290 μM to 500 μM.

A modulus is a constant or coefficient which expresses the measure of some property, such as elasticity, and can be used to relate one quantity, such as imposed force or stress, to another, such as deformation or strain.

Young's modulus, also known as elastic modulus, (E) is a material property that reflects the resistance of a material to tensile axial deformation. It is defined as the rate of change of tensile stress with tensile strain in the limit of small strains.

As opposed to axial strain, in which deformation of a plane occurs in a direction perpendicular to the plane, shear strain is characterized by deformation in a direction parallel to the plane. There is a resulting shape change without a corresponding volume change.

Shear modulus (G) is an analogous but independent material property that reflects the resistance of a material to shear deformation. It is defined as the rate of change of shear stress with shear strain at small strains.

In an exemplary embodiment, the network has a shear modulus of from 300 Pa to 50 kPa. In an exemplary embodiment, the network has a shear modulus of from 400 Pa to 30 kPa. In an exemplary embodiment, the network has a shear modulus of from 1 kPa to 25 kPa. In an exemplary embodiment, the network has a shear modulus of from 2 Pa to 17 kPa. In an exemplary embodiment, the network has a shear modulus of from 30 Pa to 50 kPa. In an exemplary embodiment, the network has a shear modulus of from 16 Pa to 45 kPa.

Exemplary materials of the invention are able to undergo a shift between a first state and a second state upon a change in their environment. For example, selected materials of the invention shift between a first state and a second state upon a change in the ambient temperature to which the material is exposed. In exemplary embodiments, one of the states more closely in resembles a natural ECM in one or more properties than the other state. For example, in functional terms, in one state a stem cell population proliferates essentially without differentiating; in the second state, the stem cell population differentiates.

As an example, a physical and/or chemical property of a network of the invention is exploited to mimic the native matrix surrounding stem cells (extracellular matrix, ECM). An exemplary property that can be manipulated is the water content of the network of the invention. Networks with differing water contents can be designed to mimic an ECM. For example, selected networks of the invention include a water content of at least about 20%, preferably, at least about 50% and still more preferably, at least about 70%. A selected hydrogel of the invention is designed to have a water content approximately that of the relevant ECM.

In another embodiment, there is provided a network that is shiftable between a first water content and a second water content. IPNs and sIPNs according to this design can be shifted between the first state and the second state, thereby controlling stem cell destiny. In general, one of the two states will more closely resemble an ECM than the other. Thus, for example, the material with the stem cells bound thereto can be shifted from the first state in which the cell population is essentially non-differentiated into the second state, more closely mimicking an ECM, inducing the stem cells to commit to a lineage. The invention also provides a material that undergoes a change in a modulus upon perturbation of its surroundings. In an exemplary embodiment, the modulus is selected from the shear modulus of the material, its tensile modulus and combinations thereof.

In an exemplary embodiment, the invention provides a material having a shear modulus of about 100 Pa to 5 kPa. Selected IPNs and sIPN have a modulus of about 50 PA in the first state and a modulus of about 400 PA in the second state. An example of a polymer that undergoes approximately this sort of phase change is a sIPN that includes a thermoresponsive polymer. The condition that promotes the first state is a temperature approximately room temperature (e.g., about 25° C.), while that promoting the second state is a temperature that is approximately human body temperature (e.g., 37° C.). HANDBOOK OF BIOMATERIAL PROPERTIES, Editors J. Black and G. Hastings, Chapman & Hall, (1998).

For example, selected IPNs and sIPNs of the invention are extremely pliable and fluid-like at room temperature (RT), but demonstrate a phase transition as the IPN or sIPN warms from RT to body temperature, yielding more rigid structures. Thus, the networks offer the benefit of in situ stabilization without the potential adverse effects of in situ polymerization (e.g., residual monomers, initiators, catalysts, etc.). The networks of the invention are preferably injectable through a syringe with about a 2 mm-diameter aperture without appreciable macroscopic fracture, are functionalized or amenable to functionalization with ligands that interact with cell surface receptors. An exemplary network is functionalized with a ligand that binds to a cell surface receptor, and the material supports cell proliferation in vitro when seeded with cells.

The networks of the invention are tunable in terms of their delivery, and dosing of a therapeutic species (e.g., stem cells). The mechanical and biochemical properties of the materials of the invention are also tunable.

In yet another exemplary embodiment, the invention provides an IPN or an sIPN that exists in a state in which it is readily deployable by minimally invasive methods. Accordingly, at room temperature (i.e., ≈20-27° C.) these IPNs or sIPNs are flowable, e.g., injectable through a small diameter aperture (from about 1 mm in diameter to about 5 mm in diameter), and are essentially free of macroscopic fracture following injection. Exemplary IPNs or sIPNs of the invention shift from the flowable state to a more rigid, less flowable state upon being heated. The shift preferably occurs at a temperature that is approximately a mammalian body temperature, e.g., 37° C.

To make a biomimetic sIPNs, a diverse array of crosslinking reagents and strategies can be used. Crosslinking exploiting orthogonal chemistry may have distinct advantages over free radical polymerization: 1) biocompatibility is increased since no free radicals are used during sIPN synthesis; 2) stem cells or other cells can be encapsulated during sIPN synthesis; and, 3) sIPN synthesis uses an “orthogonal” chemistry that is not reactive to the cell surface thereby allowing only the full ligand definition in the cell microenvironment. For example, if we activate pAAc chains with maleimide terminated grafts of EMCH, these chains can be reacted with any dithiol-containing molecule to generate a crosslinked network or sIPN. In the example below, we used di-thiol pEG and HyA chains with maleimide terminated grafts of EMCH; however, any other dithiol would suffice, including the MMP degradable peptides with a cysteine group at both ends. Candidate chemistries other than thiol-maleimide include, BrdU-thiol, phosphine-azide linkages via Staudinger ligation, and ketone-aminooxy linkages (as reviewed in Prescher and Bertozzi, Nature Chemical Biology 1, 13-21 (2005)). Also, differing chemistries at opposing ends of the crosslinking chain can be used. One example of a crosslinking chain that carries two different chemistries would be a Phosphine-Asp-Tyr-Lys-Asp-Asp-Asp-Asp-Lys-Cys peptide (phosphine-FLAG-Cys). Mixing this peptide with polymer chains that are activated with azide groups and with polymer chains activated with maleimide groups forms a gel in mild reaction conditions. Lastly, a sIPN can be grafted directly to cell receptors during sIPN synthesis by alternate chemistries if desired.

III. Pharmaceutical Compositions

In another aspect, the invention provides a pharmaceutical composition. The pharmaceutical composition includes a network of the invention. The composition may also include a delivery vehicle for the IPN or sIPN, such as a pharmaceutically acceptable diluent, carrier and the like. Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions may be formulated for a selected manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

Commonly, the pharmaceutical compositions are administered parenterally, e.g., intravenously. Thus, the invention provides compositions for parenteral administration which comprise the compound dissolved or suspended in an acceptable carrier, preferably an aqueous carrier, e.g., water, buffered water, saline, PBS and the like. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments the network of the invention can be incorporated into liposomes formed from standard vesicle-forming lipids. A variety of methods are available for preparing liposomes, as described in, e.g., Szoka et al., Ann. Rev. Biophys. Bioeng. 9: 467 (1980), U.S. Pat. Nos. 4,235,871, 4,501,728 and 4,837,028. The targeting of liposomes using a variety of targeting agents (e.g., the sialyl galactosides of the invention) is well known in the art (see, e.g., U.S. Pat. Nos. 4,957,773 and 4,603,044).

The compounds prepared by the methods of the invention may also find use as diagnostic reagents. For example, labeled compounds can be used to locate areas of inflammation or tumor metastasis in a patient suspected of having an inflammation. For this use, the compounds can be labeled with ¹²⁵I, ¹⁴C, or tritium.

IV. Methods

In another aspect, the invention provides a method of proliferating a stem cell population. This method comprises adhering the stem cell population to the network of the invention under conditions appropriate to support the proliferating.

In another aspect, the invention provides a method of differentiating a stem cell population. This method comprises adhering the stem cell population to the network of the invention under conditions appropriate to support the differentiating.

In another aspect, the invention provides a method of detaching a stem cell from the network. This method comprises adhering the stem cell population to the network of the invention, and then inducing a lower critical solution temperature phase transition in the network; thereby detaching said stem cell from the network.

Differentiated and undifferentiated cells grown on or attached to a network of this invention can be used for tissue reconstitution or regeneration in a human patient in need thereof. The stem cells are administered in a manner that permits them to graft to the intended tissue site and reconstitute or regenerate the functionally deficient area.

In an exemplary embodiment, a material of the invention that includes either undifferentiated or differentiated stem cells is administered to a patient in need of treatment for a disease that can be cured or ameliorated by the stem cells. An exemplary material according to this embodiment is one that is essentially flowable at room temperature. Upon administration to the subject, the material undergoes a change in a characteristic modulus that results in a change of state within at least a portion of the material. An exemplary change of state is one in which at least a portion of the material “stiffens,” becoming less flowable. In a further exemplary embodiment, in the second state, the modulus of the material more closely resembles the corresponding modulus in an extracellular matrix than the material in the first, flowable state.

The method of the invention can include any stem cell that is of use to treat a particular condition. In an exemplary embodiment, the method of the invention uses neural stem cells. In practice, neural stem cells and materials that include these cells, such as the sIPN of the invention can be transplanted directly into parenchymal or intrathecal sites of the central nervous system, according to the disease being treated. Grafts are done using single cell suspension or small aggregates at a density of 25,000 cells per μL (U.S. Pat. No. 5,968,829). The efficacy of neural cell transplants can be assessed in a rat model for acutely injured spinal cord as described by McDonald et al. (Nat. Med. 5: 1410 (1999)). A successful transplant will show transplant-derived cells present in the lesion 2-5 weeks later, differentiated into astrocytes, oligodendrocytes, and/or neurons, and migrating along the cord from the lesioned end, and an improvement in gate, coordination, and weight-bearing.

Certain neural progenitor cells embodied in this invention are designed for treatment of acute or chronic damage to the nervous system. For example, excitotoxicity has been implicated in a variety of conditions including epilepsy, stroke, ischemia, Huntington's disease, Parkinson's disease and Alzheimer's disease. Certain differentiated cells of this invention may also be appropriate for treating dysmyelinating disorders, such as Pelizaeus-Merzbacher disease, multiple sclerosis, leukodystrophies, neuritis and neuropathies. Appropriate for these purposes are cell cultures enriched in oligodendrocytes or oligodendrocyte precursors to promote remyelination. Accordingly, the invention provides a method of treating neural disorders using a material that includes one or more of these cell types or their progenitor(s) bound thereto.

Hepatocytes and hepatocyte precursors prepared on or adhered to a material according to this invention can be assessed in animal models for ability to repair liver damage. One such example is damage caused by intraperitoneal injection of D-galactosamine (Dabeva et al., Am. J. Pathol. 143: 1606 (1993)). Efficacy of treatment can be determined by immunohistochemical staining for liver cell markers, microscopic determination of whether canalicular structures form in growing tissue, and the ability of the treatment to restore synthesis of liver-specific proteins. Liver cells can be used in therapy by direct administration, or as part of a bioassist device that provides temporary liver function while the subject's liver tissue regenerates itself following fulminant hepatic failure. Accordingly, the present invention provides a material and a method of use for treating hepatic disorders. The material includes one or more liver-derived cell population or a progenitor thereof bound to a sIPN of the invention.

The efficacy of cardiomyocytes prepared on or adhered to a material according to this invention can be assessed in animal models for cardiac cryoinjury, which causes 55% of the left ventricular wall tissue to become scar tissue without treatment (Li et al., Ann. Thorac. Surg. 62: 654 (1996); Sakai et al., Ann. Thorac. Surg. 8:2074 (1999), Sakai et al., J. Thorac. Cardiovasc. Surg. 118: 715 (1999)). Successful treatment will reduce the area of the scar, limit scar expansion, and improve heart function as determined by systolic, diastolic, and developed pressure. Cardiac injury can also be modeled using an embolization coil in the distal portion of the left anterior descending artery (Watanabe et al., Cell Transplant. 7: 239 (1998)), and efficacy of treatment can be evaluated by histology and cardiac function. Cardiomyocyte preparations embodied in this invention can be used in therapy to regenerate cardiac muscle and treat insufficient cardiac function (U.S. Pat. No. 5,919,449 and WO 99/03973). Thus, the present invention provides a material and a method of use for treating cardiac disorders. The material includes one or more cardiac-derived cell population or a progenitor thereof bound to a network of the invention.

Drug Screening

Stem cells grown on a network of this invention can be used to screen for factors (such as solvents, drugs (e.g., small molecule drugs), peptides, polynucleotides, and the like) or environmental conditions (such as culture conditions or manipulation) that affect the characteristics of differentiated cells. In some applications, differentiated cells grown on or bound to the network of the invention are used to screen factors that promote maturation, or promote proliferation and maintenance of such cells in long-term culture. For example, candidate maturation factors or growth factors are tested by adding them to cells bound to a sIPN in different wells, and then determining any phenotypic change that results, according to desirable criteria for further culture and use of the cells.

In an exemplary embodiment, the invention provides screening applications that relate to the testing of pharmaceutical compounds in drug research. The reader is referred generally to the standard textbook “IN VITRO METHODS IN PHARMACEUTICAL RESEARCH”, Academic Press, 1997, and U.S. Pat. No. 5,030,015. Assessment of the activity of candidate pharmaceutical compounds generally involves combining the differentiated cells grown on or attached to the network of this invention with the candidate compound, determining any change in the morphology, marker phenotype, or metabolic activity of the cells that is attributable to the compound (compared with untreated cells or cells treated with an inert compound), and then correlating the effect of the compound with the observed change.

The screening may be done, for example, either because the compound is designed to have a pharmacological effect on certain cell types, or because a compound designed to have effects elsewhere may have unintended side effects. Two or more drugs can be tested in combination (by combining with the cells either simultaneously or sequentially), to detect possible drug—drug interaction effects. In some applications, compounds are screened initially for potential toxicity (Castell et al., pp. 375-410 in “IN VITRO METHODS IN PHARMACEUTICAL RESEARCH,” Academic Press, 1997). Cytotoxicity can be determined in the first instance by the effect on cell viability, survival, morphology, and expression or release of certain markers, receptors or enzymes. Effects of a drug on chromosomal DNA can be determined by measuring DNA synthesis or repair ³H-thymidine or BrdU incorporation, especially at unscheduled times in the cell cycle, or above the level required for cell replication, is consistent with a drug effect. Unwanted effects can also include unusual rates of sister chromatid exchange, determined by metaphase spread. The reader is referred to A. Vickers (PP 375-410 in “IN VITRO METHODS IN PHARMACEUTICAL RESEARCH,” Academic Press, 1997) for further elaboration.

The screening assays of the invention can be done in essentially any convenient format without limitation. In an exemplary embodiment, the invention utilizes a microarry format as described below.

Microarrays of Cells

The invention provides cells that are grown on or adhered to an IPN or sIPN of the invention. In one embodiment, the immobilized cells are formatted as a microarray that includes a plurality of addressable locations, that is functionalized with a network of the invention or a network of the invention to which a cell is bound.

Methods are known for making micro-arrays of a single cell type on a common substrate for other applications. In a simple embodiment, the wells of a microtiter plate are charged with a sample of a network of the invention to which one or more cell type population is bound. In other examples, the microarray of IPNs, sIPNs, or combinations thereof is patterned onto a substrate by photochemical resist-photolithograpy (Mrksich and Whitesides, Ann. Rev. Biophys. Biomol. Struct. 25: 55-78 (1996)). Using such methods, substrates for non-specific and non-covalent binding of certain cells have been prepared (Kleinfeld et al., J. Neurosci. 8: 4098-4120, 1988). Other methods include stamping used to produce a gold surface coated with protein adsorptive alkanethiol. (U.S. Pat. No. 5,776,748; Singhvi et al., Science 264: 696-698 (1994); Sigal et al., Anal. Chem. 68: 490-497 (1996)). Another method includes using silicone to create wells where the IPN, sIPN, or combinations thereof are patterned on the surface. The patterned silicone wells are prepared by standard photolithography to create a master onto which the silicone is cast. Methods of preparing cell arrays and acquiring data from these arrays are set forth in detail in U.S. Pat. No. 6,548,263.

In exemplary embodiments of the invention, there is provided a microarray of a single cell type. The result can be achieved by binding a single biochemically specific molecule to the micro-patterned chemical array uniformly. Thus cells bind to all spots in the array in essentially the same manner. In an exemplary embodiment, the patterned network is functionalized with a RGD motif peptide to which the stem cells bind.

In another embodiment, the invention provides a microarray that includes more than one population of cell phenotypes. The different phenotypes can array as a result of directed differentiation of the cells or it may be a result of whatever experimental conditions the cells have been subjected to. For example, if the cells are being tested for reaction to a growth factor or drug, the cells in different addressable regions of the microrray may differentiate into populations of different cell types. There may also be more than one cell type within a single addressable location.

In yet another embodiment, the microarray is functionalized with a plurality of IPNs or sIPNs bearing different cell types. A microarray according to this format provides a “library” of cell types that can be queried for the effects of various drugs, growth factors, toxins and the like.

In another aspect, the invention provides a method of optimizing a mechanical property of a network while maintaining a biochemical property of said network essentially constant, said method comprising (a) selecting an optimal value for said mechanical property; testing said mechanical property of a first said network and obtaining a first value for said mechanical property; (c) testing said mechanical property of a Xth said network and obtaining a Xth value for said mechanical property, (d) repeating step (c) until said Xth value for said mechanical property is essentially the same as said optimal mechanical value, thereby optimizing the mechanical property of the network. In an exemplary embodiment, the network is a member selected from an IPN and a sIPN. In an exemplary embodiment, the mechanical property is a member selected from shear modulus, Young's modulus, complex shear modulus, complex Young's modulus and loss angle. In another exemplary embodiment, the biochemical property is ligand density, ligand type, and method of ligand attachment.

In another aspect, the invention provides a method of optimizing a biochemical property of a network while maintaining a mechanical property of said network essentially constant, said method comprising (a) selecting an optimal value for said biochemical property; testing said biochemical property of a first said network and obtaining a first value for said biochemical property; (c) testing said biochemical property of a Xth said network and obtaining a Xth value for said biochemical property, (d) repeating step (c) until said Xth value for said biochemical property is essentially the same as said optimal biochemical value, thereby optimizing the biochemical property of the network. In an exemplary embodiment, the network is a member selected from an IPN and a sIPN. In an exemplary embodiment, the mechanical property is a member selected from shear modulus, Young's modulus, complex shear modulus, complex Young's modulus and loss angle. In another exemplary embodiment, the biochemical property is ligand density, ligand type, and method of ligand attachment.

The materials, methods and devices of the present invention are further illustrated by the examples, which follow. These examples are offered to illustrate, but not to limit the claimed invention.

### EXAMPLES

### Example 1

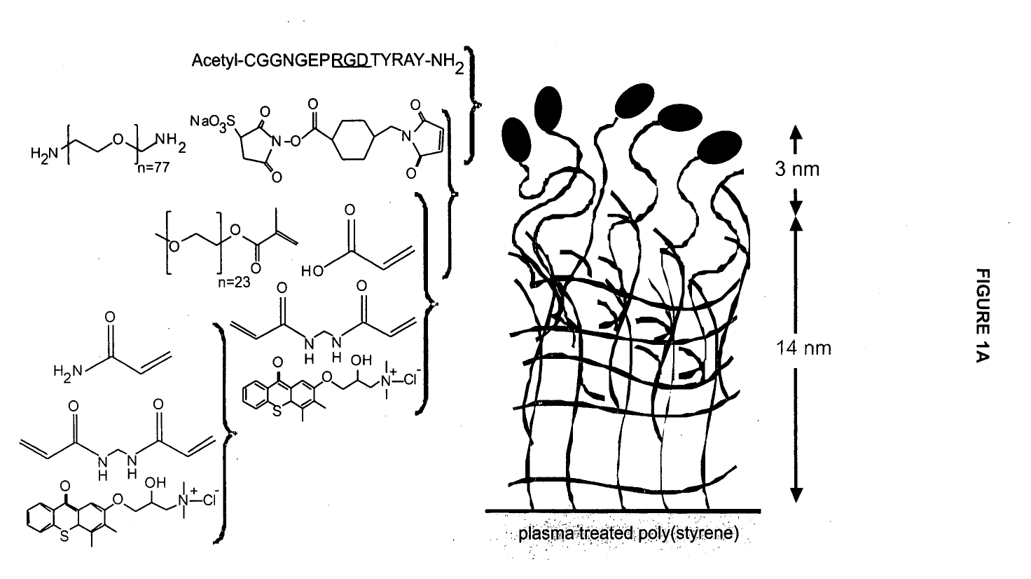

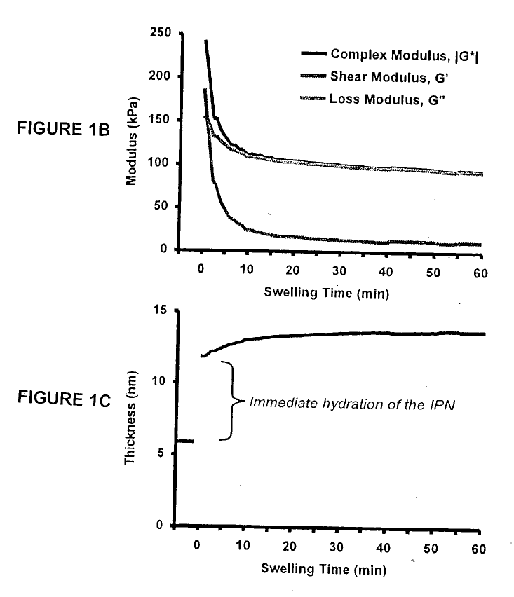

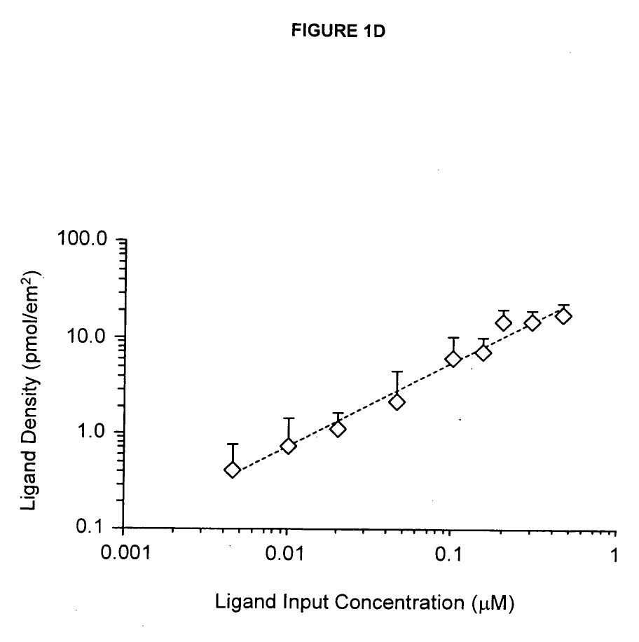

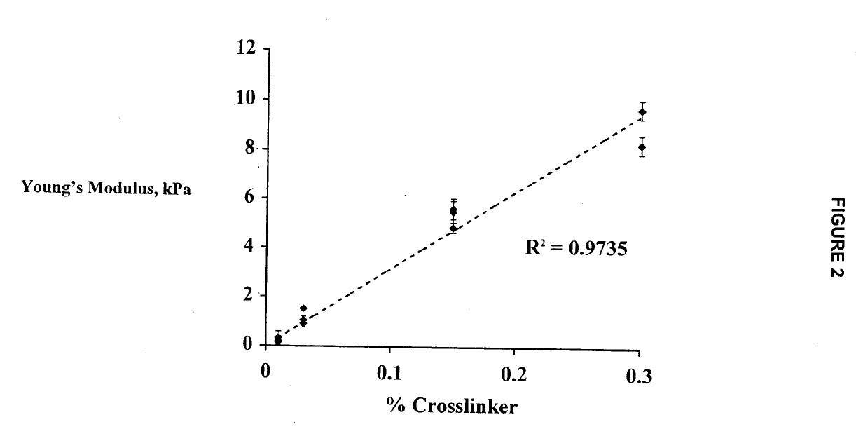

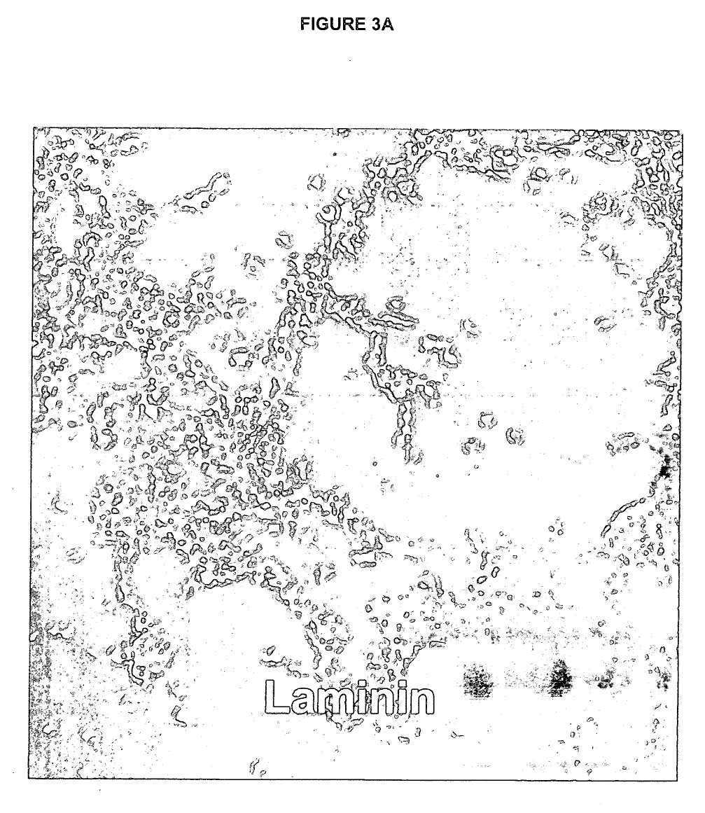

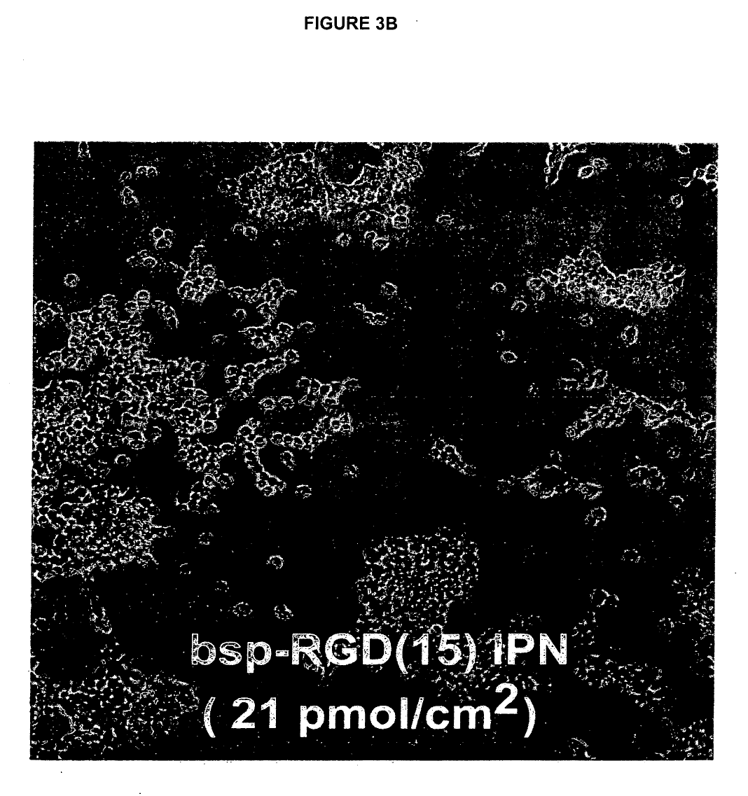

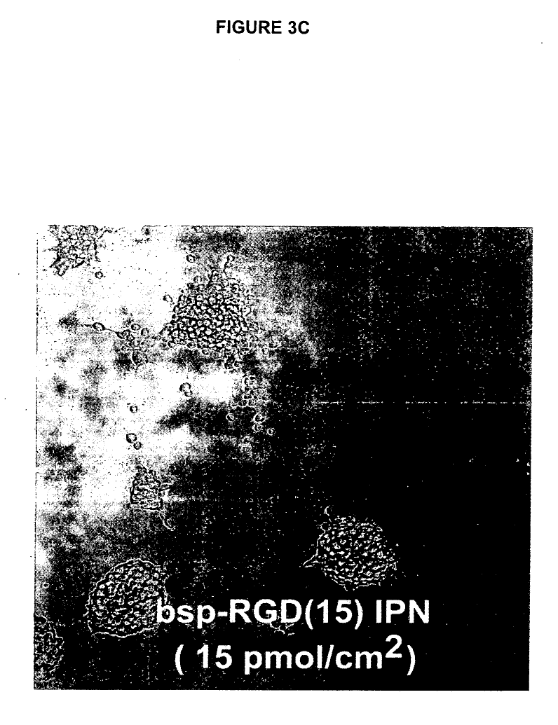

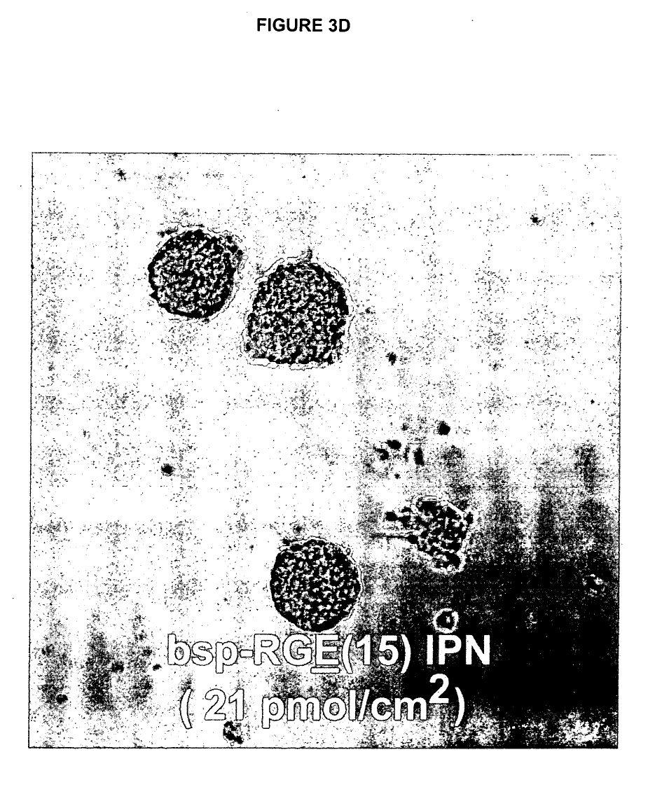

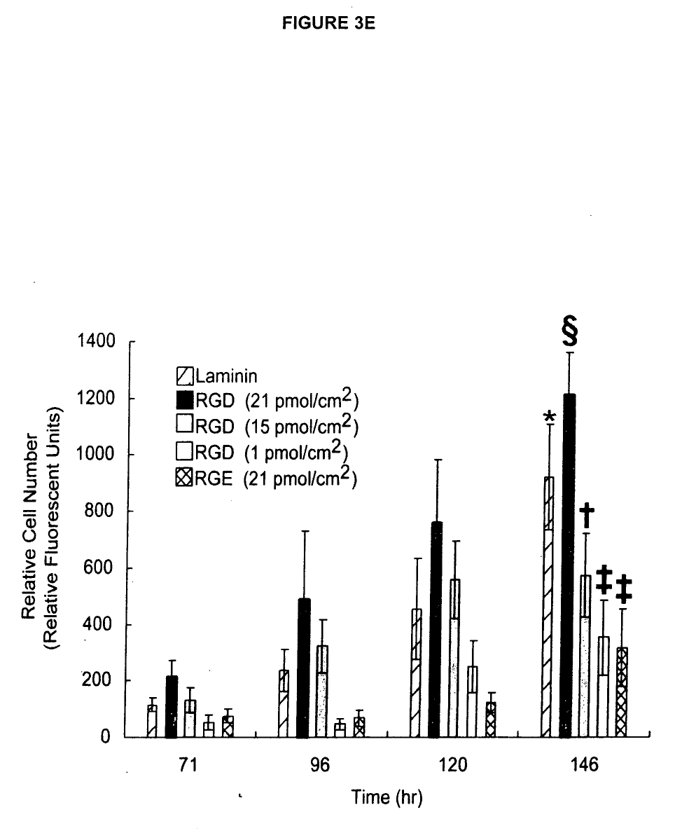

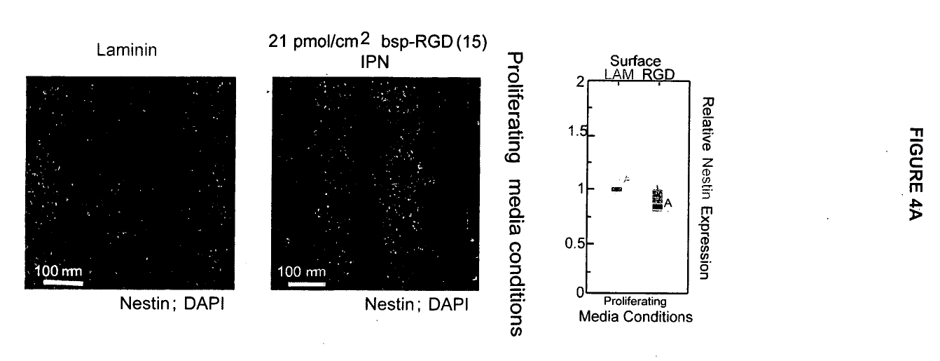

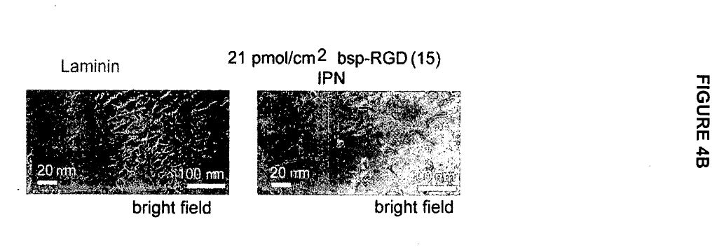

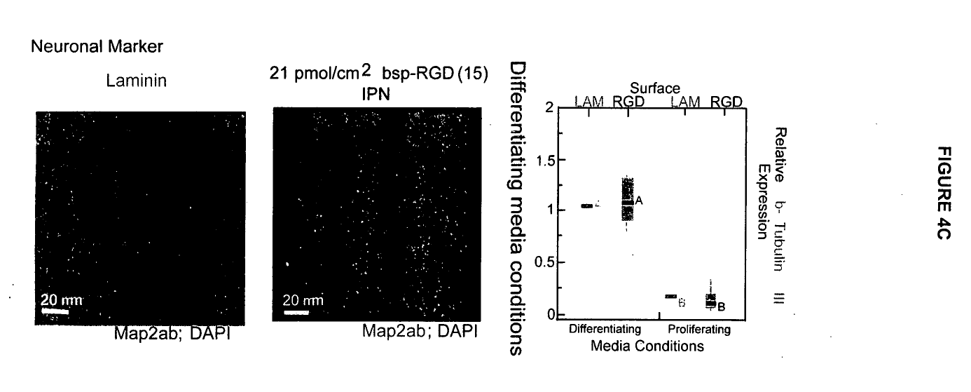

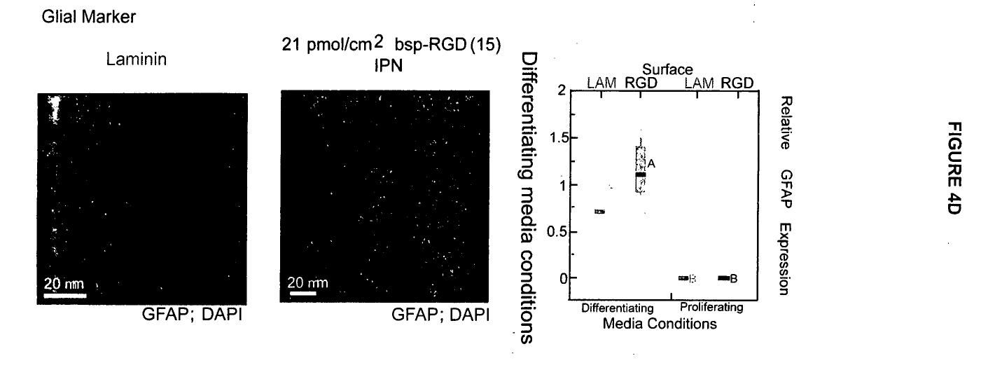

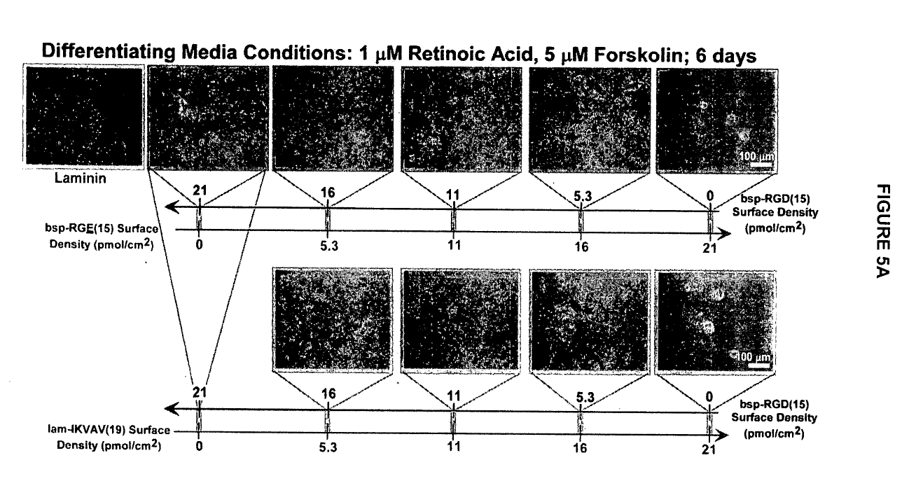

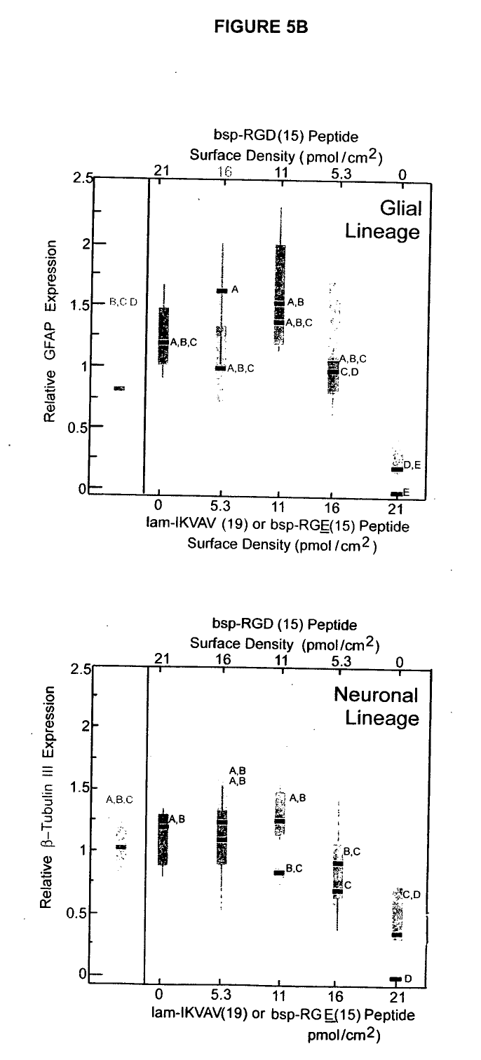

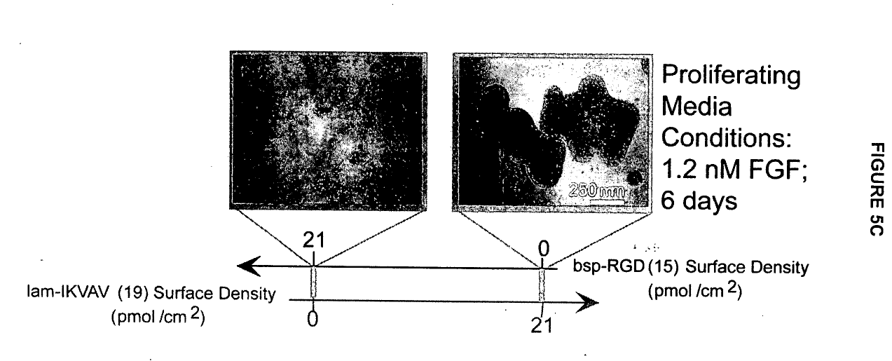

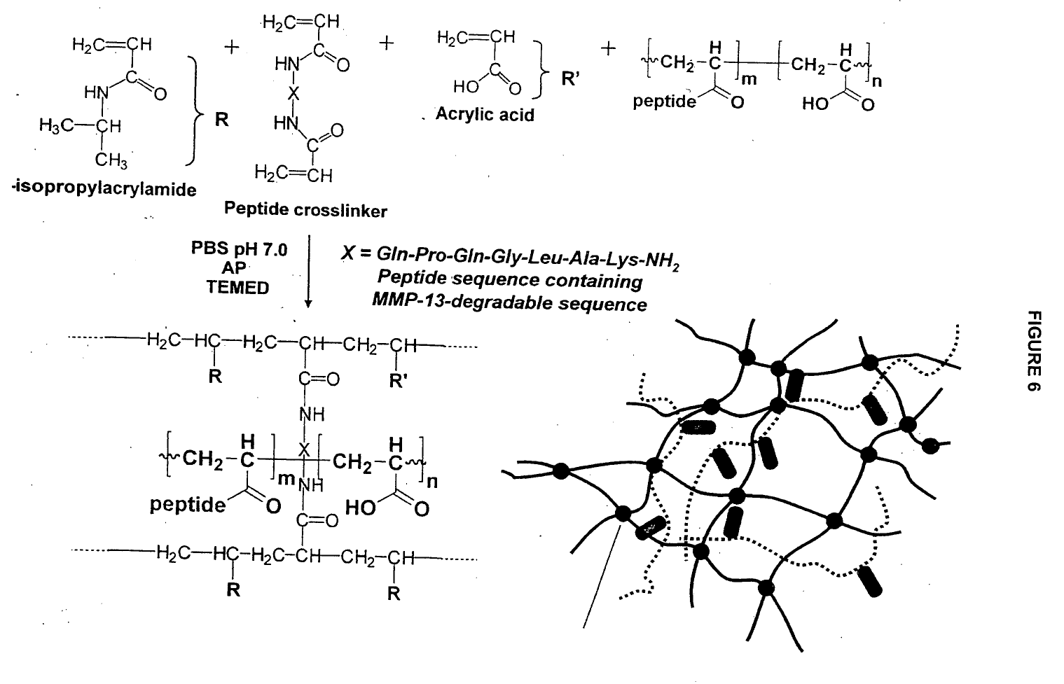

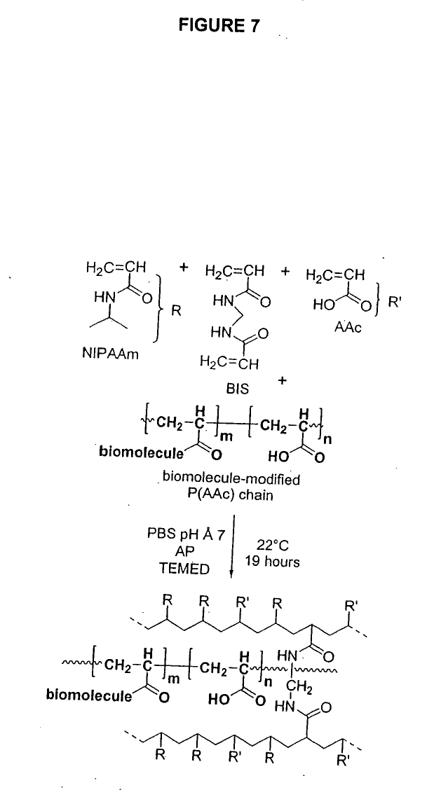

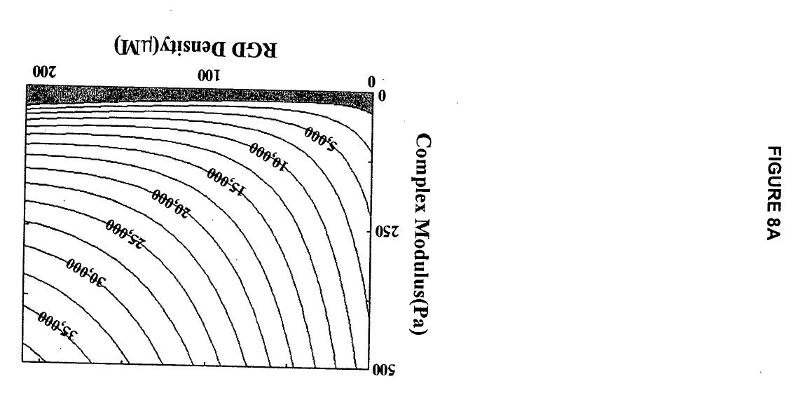

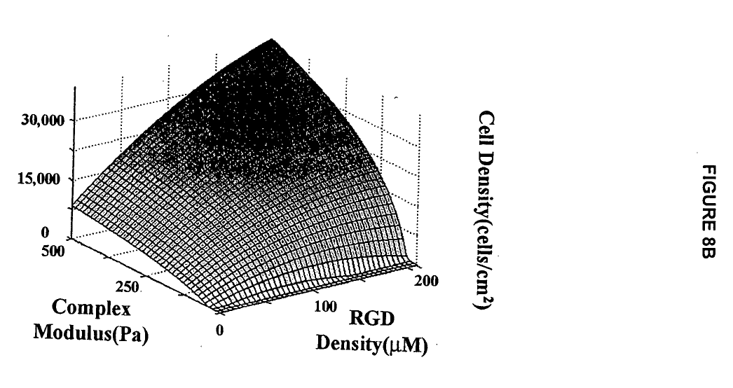

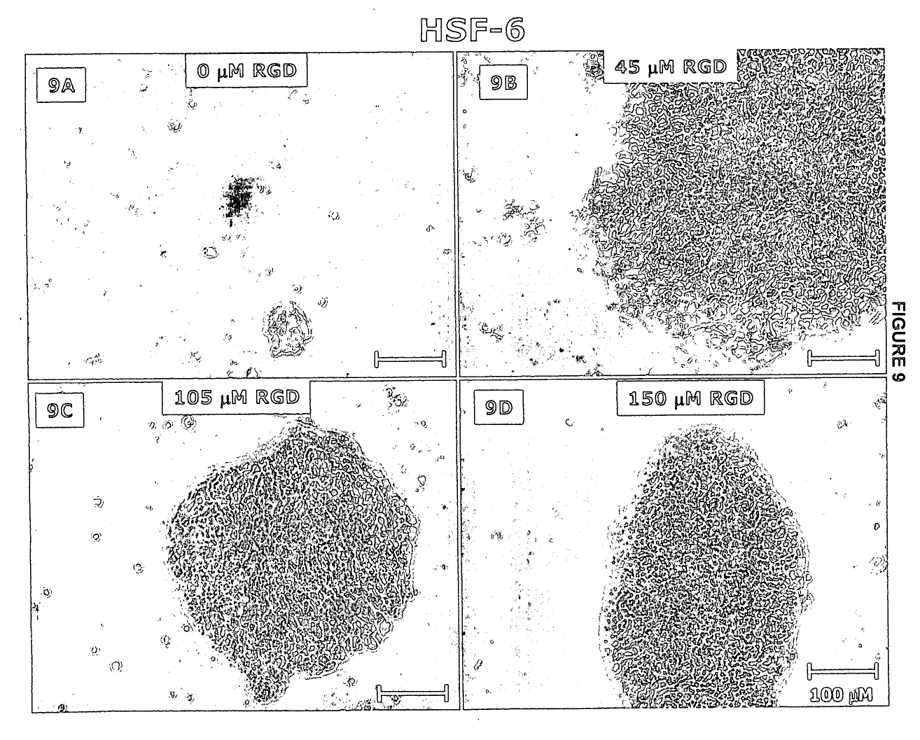

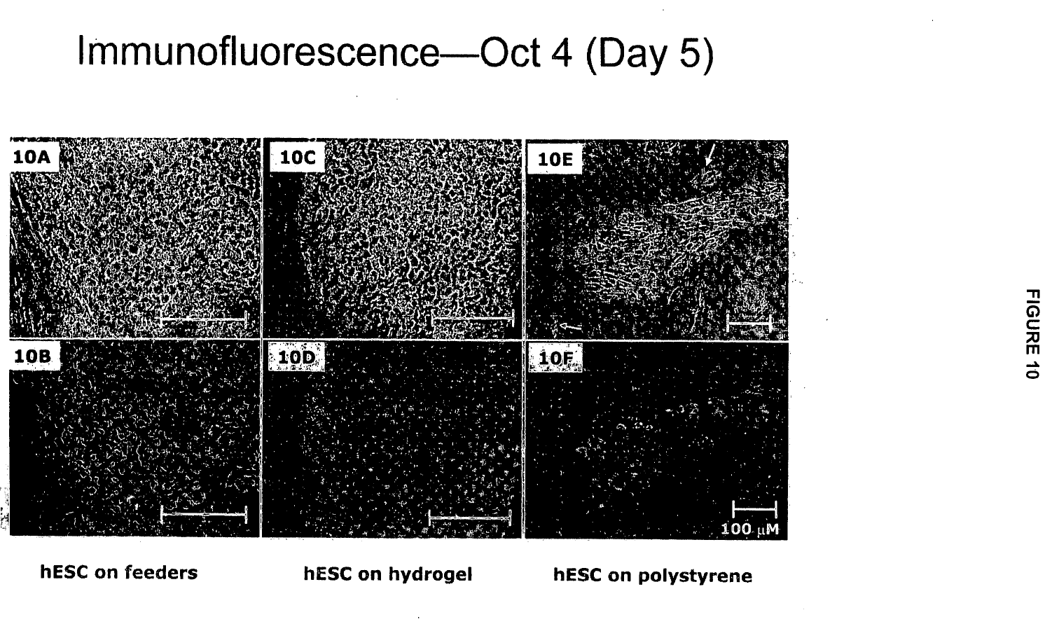

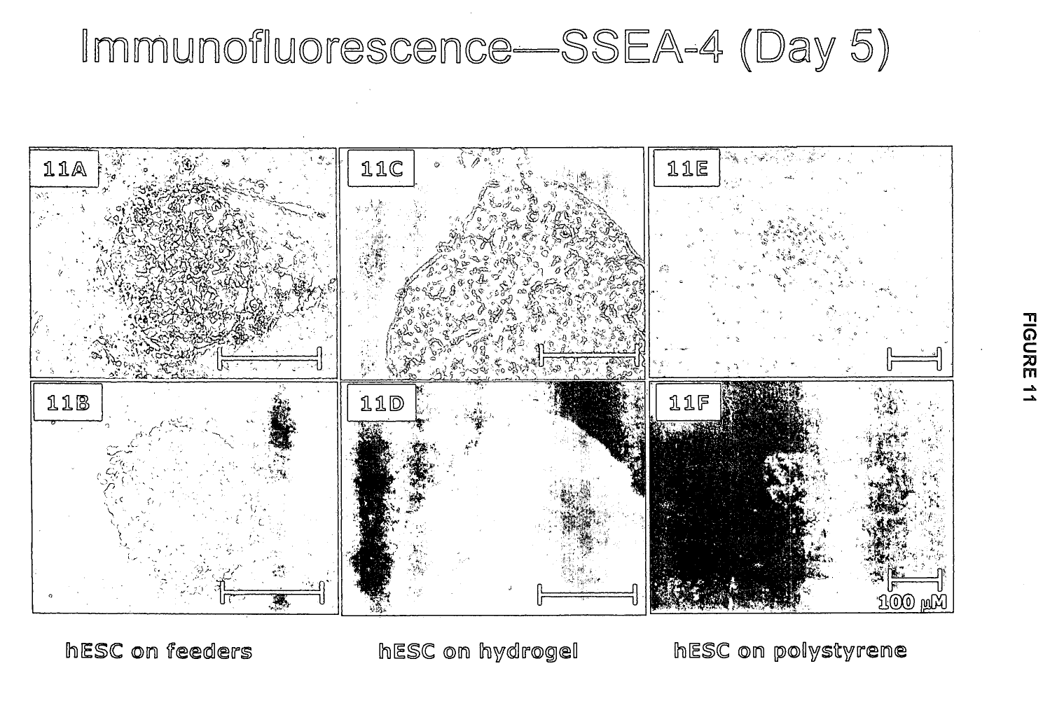

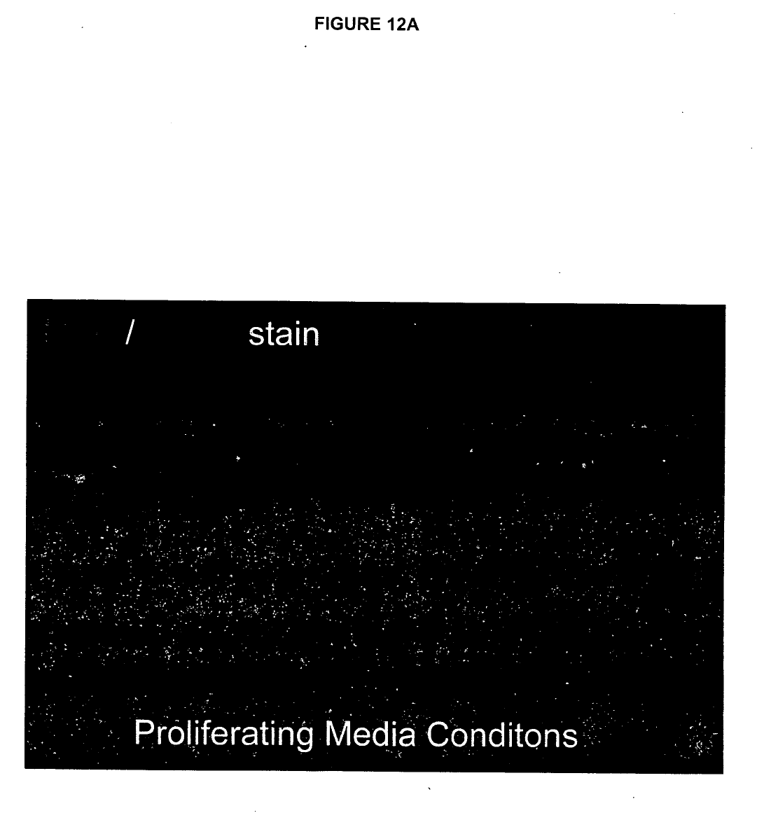

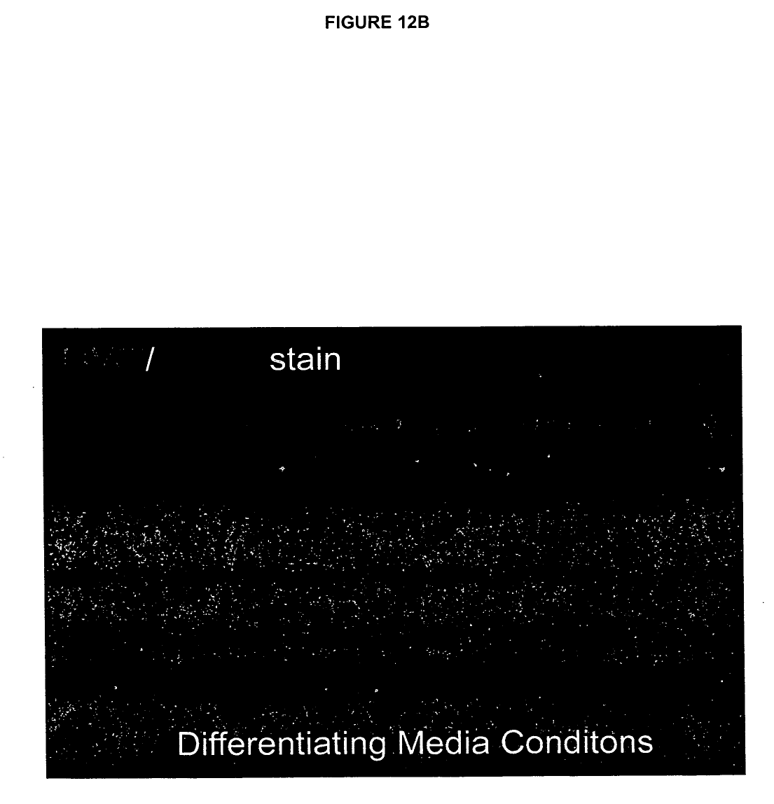

The present example details the formation of an IPN to stimulate neural stem cell proliferation incorporating bsp-RGD(15), selected from the cell-binding of bone sialoprotein (BSP), to accelerate proliferation of rat hippocamal neural stem (NSC) cells in contact with the peptide modified p(AAm-co-AAc) hydrogels. FIG. 1 provides an example of an IPN that incorporates a peptide from laminin A chain, lam-IKVAV(19).

The materials used to synthesize the IPN include the following: Acrylamide (AAm), poly(ethylene glycol) 1000 monomethyl ether monomethacrylate (PEG1000MA), acrylic acid (AAc), and N,N′-methylenebis(acrylamide) (BIS; Chemzymes ultrapure grade) were purchased from Polysciences, Inc. (Warrington, Pa.). N-hydroxysulfosuccinimide (sulfo-NHS), 2-(N-morpholino) ethanesulfonic acid, 0.9% sodium chloride buffer (MES), and sulfosuccinimidyl 4-(N-maleimidomethyl) cyclohexane-1-carboxylate (sulfo-SMCC) were acquired from Pierce (Rockford, Ill.). QTX ([3-(3,4-Dimethyl-9-oxo-9H-thioxanthen-2-yloxy)-2-hydroxypropyl] trimethylammonium chloride) was obtained from Aldrich (Milwaukee, Wis.). Allyltrichlorosilane (ATC) was obtained from Gelest (Morrisville, Pa.). Diamino-poly(ethylene glycol) [3400-PEG(NH₂)₂; 3400 g.mol⁻¹, Chromatographically pure] was purchased from Nektar (Huntsville, Ala.). All peptides were synthesized by American Peptide Co. (Sunnyvale, Calif.) and characterized using mass spectrometry and high performance liquid chromatography (purities>95%). RGD or RGE peptides were based off the integrin-binding sequence from rat bone sialoprotein: (bsp-RGD(15) peptide; bsp-RGE(15) peptide; bsp-RGD(15)-FITC) (Note that bsp-RGD(15) peptide is the same as 1-RGD as described previously (Harbers, et al., Langmuir, 21(18):8374-8384. (2005); (Harbers et al., Journal Of Biomedical Materials Research Part A, 75A(4):855-869 (2005)). The lam-IKVAV(19) peptide was from laminin A chain (amino acids 2091-2108, i.e. laminin peptide PA22-2): CSRARKQAASIKVAVSADR. Polystyrene 8-well strips (Costar #2580) and 35 mm tissue culture polystyrene dishes were purchased from Fisher Scientific (Santa Clara, Calif.). For characterization by quartz crystal microbalance with dissipation monitoring (QCM-D), quartz sensor crystals were purchased from Q-sense (Newport Beach, Calif.). All other chemicals used were reagent grade and used as purchased without further purification. All glassware was cleaned as described previously (Irwin, et al., Langmuir, 21(12):5529-36 (2005)).

The synthesis of the polymeric networks is separated into two parts: first the monomers are polymerized on a polystyrene surface to create an IPN; subsequently, the IPNs are functionalized with a biomolecule of interest. In short, AAm was crosslinked (BIS) and grafted to a oxygen plasma cleaned, polystyrene 8-well strip surface using a water soluble photoinitiator, QTX. The IPN was formed by subsequent UV-initiated polymerization of the crosslinked (BIS) network of EG/AAc. The modulus of the IPN can be controlled by adjusting the concentration of crosslinker, in either stage. A diamino-PEG spacer chain was coupled to the AAc sites using carbodiimide reaction chemistry and finally functionalized with the -RGD- peptide via a heterobifunctional cross-linker.

1.1 Synthesis of the p(AAm-co-EG/AAc) IPNs

The synthesis of the polymeric networks is separated into two parts: first the monomers are polymerized on a polystyrene surface to create an IPN; subsequently, the IPNs were functionalized with a biomolecule of interest. In short, AAm was crosslinked (BIS) and grafted to an oxygen plasma cleaned, polystyrene 8-well strip surface using a water soluble photoinitiator, QTX. The IPN was formed by subsequent UV-initiated polymerization of the crosslinked (BIS) network of EG/AAc. The modulus of the IPN can be controlled by adjusting the concentration of crosslinker, in either stage (see, Example 2). A diamino-PEG spacer chain was coupled to the AAc sites using carbodiimide reaction chemistry and finally functionalized with the -RGD- peptide via a heterobifunctional cross-linker. Polymerization and conjugation details can be found elsewhere (Harbers, et al., Langmuir, 21(18):8374-8384. (2005)), but are described briefly below.

Specifically, all reactions were carried out at room temperature unless otherwise stated. Polystyrene surfaces were cleaned by submersion in a 5 M NaOH ethanol/ASTM Reagent grade I water (water) solution (v/v, 70/30) for 1 h, rinsed, and sonicated (30 min) in water (Branson model 5510, 40 kHz, 469 W, 117 V). After cleaning, the samples were dried (N₂) and activated with an oxygen plasma. The IPN was then grafted to PS using a two-step sequential photopolymerization similar to previously published protocols. After an 8-10 min AAm solution (0.1485 g/mL AAm, 0.0015 g/mL BIS, 0.01 g/mL QTX, 0.03 mL/mL isopropyl alcohol, 0.97 mL/mL water) adsorption, the samples underwent QTX photoinitiated free radical polymerization using a transilluminator table (model TFL-40; Ultra-Violet Products, Upland, Calif.) for 4.5 minutes. The power of the table was measured at 2.3 mW/cm² using a radiometer (International Light, Inc., Massachusetts) with a band-pass filter (352-377 nm). Following polymerization, excess homopolymer was aspirated and the samples were placed in water (>10 min), rinsed, and sonicated (water, 5 min). After sonication, the samples were rinsed (water) and dried (N₂). An IPN of p(AAm-co-EG/AAc) was then formed (FIG. 1A) after the pAAm layer was exposed to an 8-10 min PEG/AAc solution (0.0200 g/mL PEG, 0.0100 g/mL BIS, 0.005 g/mL QTX, 0.0162 mL/mL, 0.5 mL/mL isopropyl alcohol, 0.5 mL/mL water) and subsequent photoinitiated polymerization for 6 minutes. Following the formation of the IPN, the samples were treated as they were after pAAm grafting.

1.2 Peptide Modification to the IPN

To functionalize the p(AAm-co-EG/AAc) IPN with biological ligands, the IPN was first equilibrated with buffer (>30 min, MES, 0.5 M, pH 7) and then 3400-PEG(NH₂)₂ spacer chains were grafted to the AAc sites via a carbodiimide reaction (60 min, MES, 0.5 M, pH 7, 0.150 g/mL 3400-PEG(NH₂)₂, 0.005 g/mL EDC, 0.0025 g/mL Sulfo-NHS). After the reaction, the solution was aspirated and the samples were rinsed 2× with 0.1 M MES buffer (pH 7.0) followed by 2× with 50 mM sodium borate buffer (pH 7.5). To couple bioactive molecules to the PEG(NH₂)₂-modified IPN, the heterobifunctional cross-linker, sulfo-SMCC, was reacted with the free amine on the PEG(NH₂)₂ chains (0.0005 g/mL Sulfo-SMCC, pH 7.5, borate buffer). The solution was then aspirated, and the samples were rinsed 2× with borate buffer followed by 2× with peptide-coupling buffer (sodium phosphate, 0.1 M, pH 6.6). Finally, the peptide containing a free thiolthe N-terminus [i.e., bsp-RGD(15), bsp-RGE(15), or lam-IKVAV(19)] was coupled (0-20 μM) to the maleimide (sulfo-SMCC). Following the reaction, the solution was aspirated and the samples were rinsed 4-5 times with coupling buffer, sonicated (water, 5 min), rinsed (water), and dried (N₂). Samples were removed at each stage and stored in an N₂ ambient environment for up to 1 year.

1.3 Characterization of IPN

To analyze the IPN chemical and mechanical properties of the IPN, X-ray photoelectron spectroscopy (XPS), fluorescently-tagged ligands, and quartz crystal microbalance with dissipation monitoring (QCM-D) were used. After each step of synthesis, XPS peak intensity ratios (i.e., ON and C/N) indicated that the IPN coated the poly(styrene) substrate, while angle-resolved studies demonstrated that the pAAm and PEG/AAc networks were interpenetrating as previously described. XPS spectra were recorded using a PHI5400 instrument (Physical Electronics, Chanhassen, Minn.) with a non-monochromatic Mg anode as the X-ray source at a takeoff angle of 55° using the same method as described elsewhere (Harbers, et al., Langmuir, 21(18):8374-8384. (2005); (Barber, et al., Biomaterials, 26(34):6897-905 (2005)).

IPN physical properties, specifically thickness as well as shear storage and loss moduli, were measured by modeling QCM-D frequency and dissipation changes upon swelling of the IPN in phosphate buffered saline (PBS) (Irwin, et al., Langmuir, 21(12):5529-36 (2005)) (FIG. 1b-c). Upon exposure to PBS, the IPN swelled immediately to ˜12 nm and was non-fouling (i.e., low protein adsorption) to media components (Irwin, et al., Langmuir, 21(12):5529-36 (2005)). The surfaces of the QCM-D sensor crystals were modified for characterization with an IPN of p(AAm-co-EG/AAc) as described above, except that a unsaturated silane was chemisorbed to the surface prior to the polymerization step as described previously (Irwin, et al., Langmuir, 21(12):5529-36 (2005)). Briefly, sensor crystals are coated with 200 m of silicon/silicon dioxide (Si/SiO₂), and then an unsaturated organosilane, ATC, was grafted onto the Si/SiO₂ surfaces by soaking them in a 1.25% (v/v) solution of ATC in anhydrous toluene (prepared in a glovebox) for 5 min. After baking them for 30 min at 125° C., the IPN synthesis of p(AAm-co-EG/AAc) proceeded as described above. A QCM-D D300 (Q-sense) was used in this study, as described in detail elsewhere (Irwin, et al., Langmuir, 21(12):5529-36 (2005)). Briefly, in a QCM-D experiment, four separate resonant frequencies (overtones, n) were used to drive oscillation of the shear wave through the crystal: ˜5 MHz (fundamental overtone, n=1), ˜15 MHz (n=3), ˜25 MHz (n=5), and ˜35 MHz (n=7). The applied voltage for each resonant frequency was sequentially pulsed across the sensor crystal, allowing shear wave dissipation with the simultaneous measurement of the absolute dissipation (D) and the absolute resonant frequency (f) of the crystal for all four overtones. All measurements were taken at 37° C. The f and D values were recorded for the crystals before and after ex situ modification both dry and in PBS. Dry thickness was calculated via the Sauerbrey relationship, ΔM=−C.Δf.n⁻¹, where ΔM was the total change in mass of a rigid, elastic adlayer, C was a 17.7 ng.cm⁻¹.Hz⁻¹ constant based on the physical properties of the quartz crystal, and n was the overtone number. The IPN surfaces were swollen in PBS (sample size of 3). Degassed PBS was introduced into the measurement chamber, and the chamber was sealed shut during the 16 hr swelling period. For protein adsorption studies, proliferation or differentiation media (see neural stem cell culture) was introduced for 1 hr, and then rinsed twice with PBS for 5 min.

FITC-labeled peptides were used in several IPN preparations to determine the surface density of bioactive peptides as a function of the amount of soluble peptide added to the surface conjugation reaction (data not shown), which allowed subsequent fine-tuning of peptide surface density. Peptide density and degradation analysis of such surfaces have been characterized elsewhere (Harbers, et al., Langmuir, 21(18):8374-8384. (2005); (Harbers et al., Journal Of Biomedical Materials Research Part A, 75A(4):855-869 (2005)) (Irwin, et al., Langmuir, 21(12):5529-36 (2005); (Barber, et al., Biomaterials, 26(34):6897-905 (2005)).

The density of a biologically relevant ligand was measured after grafting to the IPN. A fluorescence assay was developed to quantify ligand density on IPN modified surfaces. (Harbers, et al., Langmuir, 21(18):8374-8384. (2005)). Samples were modified by substituting bsp-RGD(15)-FITC for bsp-RGD(15). Surfaces lacking the SMCC cross-linker were used as controls to ensure that signal from entrapped or non-specifically adsorbed fluorophore could be subtracted as background. Following the IPN synthesis, samples were dried (N₂) and either stored under nitrogen or immediately prepared for measurement. To improve quantum efficiency, 10 μl of ligand coupling buffer were added to each dried sample well to form a hydrated thin IPN. Samples were then inverted and immediately read using a Spectramax GeminiXS spectrofluorometer (Molecular Devices, CA; ex/em/cutoff, 485/538/530 nm)). Density standards were generated by adding 50 μL of RGD-FITC solutions prepared in water to PEG(NH₂)₂ modified wells and drying under vacuum for >2 hrs to form a dried film of known ligand density (0.11 to 37.15 pmol/cm²). After drying, density standards were treated the same as experimental wells. Figure xx shows the ligand density data for RGD-FITC coupled to the IPN surface as a function of input concentration. Figure xx represents the data on a log-log scale demonstrating the linear control of ligand density based on solution input concentration. These results demonstrate that ligand density saturated at ≈20 pmol/cm² at input concentrations ≧0.46 mM. These results are in agreement with an independent fluorescent density measurement technique that relies on enzymatic cleavage and subsequent release of the surface bound FITC labeled peptide into solution. (Harbers et al., J Biomed Mater Res A, (2005)). Given the close agreement between these two independent methods, the fluorescent technique used was an effective, sensitive, and simplistic method to measure ligand densities on the IPN.

Therefore, the peptide-modified IPN ligand density (1.2-21 pmol/cm²), hydrated thickness (14 nm), swelling behavior (polymer volume fraction, v₂ₛ=0.43), complex shear modulus (|G*|=94 kPa), and non-fouling properties define a specific cellular microenvironment, namely by specifying the dose and mechanical context of the chemical signals presented to stem cells.

### Example 2

This example details the creation of IPN coatings of varying stiffness to investigate the combined effects of substrate modulus and ligand density on stem cell self-renewal and fate determination. The materials used in this synthesis were the following: methacryloxypropyltrimethoxysilane (MPMS) obtained from Gelest (Morrisville, Pa.); acetic acid (AA), acrylamide (AAm), bisacrylamide (Bis), N,N,N′,N′-tetramethylethylenediamine (TEMED), poly(ethylene glycol) monomethyl ether monomethacrylate, MW 1000) (PEGMA), camphorquinone (CQ), acrylic acid (AAc), and 3400 MW diamino-PEG [PEG(NH₂)₂] obtained from Polysciences (Warrington, Pa.); ammonium persulfate (AP), methanol (MeOH), and dichlorodimethylsilane (CMS) obtained from Sigma-Aldrich (St. Louis, Mo.); 1-ethyl-3-[3-dimethylaminopropyl]-carbodiimide hydrochloride (EDC), N-hydroxysulfosuccinimide (Sulfo-NHS), and Sulfosuccinimidyl-4-(N-maleimidomethyl)-cyclohexane-1-carboxylate (sulfo-SMCC) obtained from Pierce (Rockford, Ill.); and bsp-RGD(15) from American Peptide (Sunnyvale, Calif.).

The IPN coating was polymerized in two parts: first an AAm layer was polymerized directly on quartz discs, and next a poly(ethylene glycol/acrylic acid) (PEG/AAc) layer was polymerized within the AAm network. The IPNs were then modified with an RGD cell-binding peptide isolated from bone sialoprotein to allow for cell attachment. Quartz discs (1″ O.D.×¼″ thick; Chemglass, Inc) were cleaned with an oxygen plasma (March Plasmod; Concord, Calif.) for 5 min at 1 Torr. The discs were functionalized with an organosilane, MPMS, by immersing in a solution composed of 94% (v/v) MeOH, 5% (v/v) water, 1% (v/v) MPMS, and 1 mM AAm for 5 min, rinsed in MeOH, and baked for 30 min at 110° C. Solutions of 10% AAm and 0.01-0.3% Bis were prepared in water and degassed. Polymerization was initiated with AP and TEMED. AAm solutions were pipetted onto functionalized quartz discs and sandwiched with top coverslips that were been modified with CMS. After polymerization, the samples were immersed in water, and top coverslips were removed carefully. A second layer of PEG/AAc was polymerized on top of and within the AAm layer by previous methods (Bearinger et al., Journal of Biomaterials Science-Polymer Edition 9(7):629-652). The AAm-modified quartz discs were allowed to equilibrate in a solution of 0.02 g/mL PEGMA, 0.01 g/mL Bis, 0.3348 g/mL CQ, and AAc in methanol for 5 min. The PEG/AAc layer was polymerized in a light box (Rayonet; Branford, Conn.) for 40 min, and samples were rinsed in methanol and water.

The surfaces were then functionalized with an RGD cell-binding peptide. PEG spacer chains were tethered to the AAc sites in the PEG/AAc layer by exposure to a solution of 0.20 g/mL of PEG(NH₂)₂, 0.4 mg/mL EDC, and 1.1 mg/mL Sulfo-NHS for one hour. Next, a heterobifunctional crosslinker, sulfo-SMCC (0.5 mg/mL in sodium borate buffer, pH 7.5, 30 min) was used to attach a cell-binding RGD peptide (0.1M solution in sodium borate buffer, pH 6.6, reacted overnight).

Atomic Force Microscopy (AFM) Experiments were performed in order to measure the Young's modulus (E) of the gels. A Bioscope AFM in force-mode and a fluid cell were used in these experiments. A v-shaped silicon nitride tip was modified with a 10 um polystyrene bead in order to reduce strain on the gels during measurements. The E of the gels varied linearly from 0.23±0.09 kPa to 9.86±0.14 kPa depending on the concentration of BIS used in the polymerization of the AAm layer. Data depicting this behavior is presented in FIG. 2, where the square of the correlation coefficient (R²) is 0.9735.

### Example 3

IPN seeded with Growth Factors and Satellite cells

Cell Culture and Seeding. Four-month-old B6.129S7-Gt(ROSA)₂₆Sor/J mice (The Jackson Laboratory) are killed, and the satellite cells are isolated from hindlimbs, as described in Irintchev et al., Eur. J. Neurosci., 10:366 (1998). Briefly, hindlimb skeletal musculature are surgically excised, finely minced, and disassociated in 0.02% Trypsin (GIBCO) and 2% Collagenase type 4 (Worthington) for 60 min at 37° C./5% CO₂ while agitating on an orbital shaker. Disassociated muscle can be strained in a 70-μm sieve, centrifuged at 1,600 rpm (Eppendorf 5810R) for 5 min, and resuspended in 10-mL-high glucose DMEM, supplemented with pyruvate (GIBCO). Media is further supplemented with 10% FBS and 1% penicillin/streptomycin (GIBCO). Resuspended cells are plated on an IPN of the invention, such as described in Example 1, and HGF (50 ng/mL) and FGF2 (50 ng/mL) are added to the medium. After 7 days, cultures are passaged, and purified satellite cell suspensions are obtained via Percoll fractionation, as described in McKinney-Freeman et al., Proc. Natl. Acad. Sci. USA, 99: 1341-1346, (2002). Purified cultures a incubated for 7 days at 37° C. until 80% confluent and then collected via trypsinization and seeded at 10⁷ cells/ml onto an modified open-pore polymer scaffolds.

### Example 4

In this study, rat adult neural stem cells (NSCs) were grown on an IPN consisting of two crosslinked polymer networks, one of poly(acrylamide) and the other of poly(ethylene-co-acrylic acid) [(p(AAm-co-EG/AAc)]. In addition, (bsp-RGD 15) was grafted via the acrylic acid sites on the p(AAm-co-EG/AAc) IPN to provide cell binding domains. An important feature of this IPN is that ligand density is easily tunable by varying the concentration of [bsp-RGD(15)] peptide during grafting. Furthermore, ligand density is completely defined for the culturing surface, as the non-fouling nature (i.e., low protein adsorption) to media components of the remainder of the IPN [i.e., p(AAm-co-EG) IPN] has been extensively characterized (Harbers, et al., Langmuir, 21(18):8374-8384. (2005); (Bearinger et al., Journal of Biomaterials Science-Polymer Edition, 9(7):629-652(1998)). Examples 1 and 2 describe the synthesis and characterization of bsp-RGD(15)-modified IPNs. After synthesis, IPNs were sterilized by the use of ethanol as previously described (Huebsch et al., J Biomed Mater Res B Appl Biomater, 74(1):440-7 (2005)).

As a positive control in this study, cell culture surfaces were coated with an ECM protein, laminin, using traditional stem cells culturing protocols. The positive control surfaces were coated with poly-ornithine and saturated with mouse laminin I (Invitrogen, from the Engelbreth-Holm-Swarm (EHS) sarcoma) as described in the literature (Lai, K., et al., Nat Neurosci, 6(1):21-7 (2003)). Briefly, poly-ornithine (10 μg.mL⁻¹ in water) was added to cover a polystyrene culture well (˜50 μL) and incubated overnight at room temperature. Wells were then rinsed twice with sterile water, and laminin (˜5 μg.mL⁻¹ in phosphate buffered saline) was added to cover the well. After incubation overnight at 37° C., wells were frozen at −20° C. until use.

As a negative control in this study, IPNs grafted with bsp-RGE(15) were used to test the specificity of cell response to the RGD motif in bsp-RGD(15)-modified IPNs.

4.1 NSC Isolation and Culturing Conditions

Neural stem cells were isolated from the hippocampi of adult female Fischer 344 rats as previously described (Lai, K., et al., Nat Neurosci, 6(1):21-7 (2003)). Cells at (200-10,000 cells/well) were seeded onto peptide-modified IPNs and laminin-modified culture wells and incubated (37° C., 5% CO₂) in serum-free media consisting of DMEM/Hams F-12 medium with N-2 supplement. These media conditions were supplemented with various soluble factors to modulate cell behavior: 20 ng.ml⁻¹ basic fibroblast growth factor (bFGF) for cell proliferation or 1 μM retinoic acid with 5 μM forskolin for neuronal differentiation. Wells were rinsed every 48 hrs with fresh media.

4.2 NSC Proliferation on bsp-RGD(15)-Modified IPNs

NSCs isolated from the adult hippocampus were seeded onto bsp-RGD(15)-modified IPNs at various cell densities over four orders of magnitude. Under media conditions that include a factor critical for self-renewal, bFGF (i.e., proliferating media conditions), cell adhesion and morphology on the RGD surfaces were similar to that on laminin (FIG. 2 a-b). By contrast, on surfaces with either low or no bsp-RGD(15), cells did not adhere effectively (FIG. 2 c-d) and resembled NSC growth in suspension as neurospheres Sen et al., Biotechnol Prog. 18(2):337-45 (2002)). Such spheres provide less precise control over the cellular microenvironment, due in part to spatial gradients in signaling and nutrients and internal necrosis. The bsp-RGE(15), which differs from the bsp-RGD(15) peptide by only a methylene group, did not support attachment and thus highlighted the specificity of the NSC engagement with the peptide-modified IPN.

For quantitative assays of proliferation, the NSCs were seeded at 1000 cells per well on various surfaces and grown for 3-6 days, and cell number was determined using a fluorescent dye that binds to nucleic acids, CyQUANT (Molecular Probes, Eugene, Oreg.). Briefly, cells grown on a particular surface for a fixed duration were washed once with phosphate buffer saline and lysed in the manufacturer's buffer with dye. Next, the fluorescent intensity of resulting solution was measured. Importantly, the bsp-RGD(15)-modified IPN also supported NSC proliferation in a ligand dose-dependent fashion, and IPNs with the highest bsp-RGD(15) density supported faster cell proliferation than standard laminin-coated surfaces (FIG. 2e). Any increase in cell number on the negative control bsp-RGE(15)-modified IPNs reflected growth of weakly adherent neurospheres (FIG. 2d-e). About 10 pmol.cm⁻² bsp-RGD(15) was needed to support proliferation of NSCs, corresponding to ˜10⁶ ligands per cell for the 10 μm diameter cells.

4.3 NSC Phenotype and Differentiation on bsp-RGD(15)-Modified IPNs

In addition to precise control of cell proliferation, the bsp-RGD(15)-modified IPNs supported multipotent NSCs in several states of differentiation. To assay phenotype, two methods were used: quantitative real time PCR (qRT-PCR) and immunofluorescent staining. These methods have been frequently used to assay phenotype of cells (Abranches, et al., Biotechnol Appl Biochem, 44(Pt 1): 1-8 (2006)). In these experiments, NSCs seeded onto bsp-RGD(15)-modified IPNs at 10,000 cells/well and the media conditions either promoted self-renewal, 1.2 nM bFGF (i.e., proliferating media conditions) or differentiation, 1 μM retinoic acid with 5 μM forskolin for neuronal differentiation. For immunofluorescent staining, cells on days 1-14 were fixed with 4% paraformaldehyde and stained with primary antibodies of mouse anti-nestin (1:1000 dilution), mouse anti-microtubule associated protein 2ab (Map2ab) (1:250), and guinea pig anti-glial fibrillary acidic protein (GFAP) (1:1000). cytoskeletal markers that are characteristic of a particular differentation state. Nestin is a marker of an immature neural cell, Map2ab marker of differentation to a neuron, and GFAP is a marker of differentiation into a glial phase or an astrocyte. Detection of primary antibodies was performed with Alexa fluorochrome-conjugated secondary antibodies at a dilution of 1:250. Nuclei were stained with the nuclear marker Sybergreen and 4′-6-Diamidino-2-phenylindole (DAPI) (Molecular Probes, Eugene, Oreg.). Images were collected on an Olympus IX-50 microscope and Zeiss META 510 confocal microscope. Quantitative real time PCR was used as a complementary technique to accurately quantify specific cDNA concentrations in various cDNA samples from cells grown on IPNs and laminin (using a Bio-Rad Laboratories iCycler). GFAP expression levels were quantified as a marker for astrocytic differentiation of the progenitor cells. β-Tubulin-III was used as a marker for neurons. Nestin was used as a marker for NSCs. Ribosomal 18S was employed to normalize the various samples for differences in the starting amounts of cDNA used in each sample. The utilized primers and TAQMAN probes are listed as follows in the following format (marker, left primer, right primer, hybridization TAQMAN oligo):

(GFAP, GACCTGCGACCTTGAGTCCT,
TCTCCTCCTT-GAGGCTTTGG,
TCCTTGGAGAGGCAAATGCGC),
(β-Tubulin-III, GCATGGATGAGAT-GGAGTTCACC,
CGACTCCTCGTCGTCATCTTCATAC,
TGAACGACCTGGTGTCTGAG)
(Nestin, GAGCTCTCTGGGCAAGTGGA,
CTCCCACCGCTGTTGATTTC,
AGGACAG-TCAGCAGTGCCTGCA),
and
(18S, GTAACCCGTTGAACCCCATTC,
CCATCCAATC-GGTAGTAGCGA,
AAGTGCGGGTCATAAGCTTGCG).

Standards for performing qRT-PCR were pPCR4-TOPO plasmids (Invitrogen) containing the containing the amplicon of interest as an insert. The plasmids were linearized by restriction digest and quantified by absorbance, and tenfold serial dilutions from 1 ng/μL to 10⁻⁹ ng/μL were prepared to generate a standard curve. All samples were conducted in duplicate.

Similar protein levels of nestin, a neurofilament characteristic of immature neural cells (Lendahl et al., Cell, 60(4): 585-95 (1990)), were observed on bsp-RGD(15)-modified IPNs and laminin surfaces for all time points analyzed up to 14 days in bFGF (i.e. proliferating conditions) (FIG. 3a). Subsequently, cells were subjected to differentiation conditions (i.e. retinoic acid and forskolin) (Palmer et al., T. D., Mol Cell Neurosci, 6(5):474-86 (1995)). Cell morphology as well as immunostaining of lineage specific markers were similar on laminin versus bsp-RGD(15)-modified IPN surfaces (FIG. 3b-d, left). Furthermore, quantitative RT-PCR for lineage specific markers indicated that the laminin and bsp-RGD(15)-modified IPN surfaces supported differentiation into neural lineages to the same extent (FIG. 3b-d, right). We next examined whether cell differentiation depended on RGD density, as found previously for cell proliferation (FIG. 2). The ability of the surfaces to support differentiation decreased with reducing RGD density (FIG. 4a-b). Between 5.3 and 11 pmol.cm⁻² bsp-RGD(15) was needed to support both proliferation and differentiation (see below) of NSCs.

This examples indicate that a synthetic IPN presenting a simple RGD-containing motif functionally replaced the ability of laminin I to support cell attachment, proliferation, and differentiation, a significant result considering that complex ECM molecules such as laminin are extremely large (850 kDa) and contain a number of cell-binding motifs (Tashiro, et al.,. J Cell Physiol, 146(3):451-9 (1991); (Bellamkonda et al., J Neurosci Res, 41(4): 501-9 (1995), (Powell et al., Int J Biochem Cell Biol, 29(3): 401-14 (1997)).

### Example 5

In this study, we took advantage of the fact that the highly modular synthetic IPN network could be conjugated with diverse combinations of biochemical signals at various ratios. Rat adult neural stem cells were grown on an IPN with a mixture of two different peptides. The IPN consisted of two crosslinked polymer networks, one of pAAm and the other of PEG/AAc. In addition, a mixture of peptides were grafted via the acrylic acid sites on the p(AAm-co-EG/AAc) IPN to engage and potentially influence differentiation of the NSCs. The mixture consisted of any two of the following peptides: [bsp-RGD(15)], 19 amino-acid laminin peptide putatively involved in promoting neurite outgrowth of mature neurons and differentiation of fetal neuronal progenitors (Tashiro, et al., J Biol Chem, 264(27): 16174-82 (1989); (Bellamkonda et al., J Neurosci Res, 41(4): 501-9 (1995); (Silva, et al., Science, 303(5662): 1352-5 (2004)) CSRARKQAASIKVAVSADR [lam-IKVAV(19)], and bsp-RGE(15). Example 1 describes the synthesis and characterization of the peptide-modified IPN. NSC isolation, culturing conditions, and differentiation assays were performed as in Example 4.

We observed that lam-IKVAV(19) did not enhance either cell proliferation or differentiation (FIG. 4b-c). On pure lam-IKVAV(19)-modified IPNs, NSCs did not adhere under differentiating or proliferating media conditions, similar to behavior on the negative control RGE surface (FIG. 1d, FIG. 4a-c). Furthermore, cell differentiation into either a neuronal or astrocytic lineage progressively decreased as the IKVAV/RGD ratio increased (FIG. 4a-b). These results further confirm that the RGD peptide-modified IPN, without introducing any cooperative effects from mechanisms involving lam-IKVAV(19), was able to functionally substitute for laminin in early differentiation stages of adult NSCs.

### Example 6

Method for Stem Cell Recovery Without Using Enzymes for IPNs.

Human ESCs can be grown and recovered on thermoreversible IPNs grafted to glass, quartz, other metal oxides, or polystyrene. These thermoreversible IPNs can be made with variable modulus and ligand surface densities to control stem cell self-renewal and fate. Exploiting the thermoreversible nature of the IPN, the undifferentiated stems can be removed from the substrate by simply adjusting the thermal environment (i.e., reducing the ambient temperature below the LCST of the IPN). Culturing stem cells under these conditions alleviates the aforementioned contamination problems associated with feeder layers and use of animal derived products such as enzymes. Synthesis of the thermoreversible IPNs grafted to quartz is given as an example of this method. The materials used in this synthesis are: methacryloxypropyltrimethoxysilane (MPMS) obtained from Gelest (Morrisville, Pa.); acetic acid (AA), NIPAAm, methoxy poly(ethylene glycol) (MW=200) methacrylate (mPEG200MA) (MW=300 g/mol), poly(ethylene glycol) (MW=200) diacrylate (PEG200DA) (MW=302 g/mol), N,N,N′,N′-tetramethylethylenediamine (TEMED), poly(ethylene glycol) monomethyl ether monomethacrylate, MW 1000) (pEG₁₀₀₀MA), camphorquinone (CQ), acrylic acid (AAc), and 3400 MW diamino-PEG [3400-PEG(NH₂)₂] obtained from Polysciences (Warrington, Pa.); ammonium persulfate (AP), methanol (MeOH), and dichlorodimethylsilane (CMS) obtained from Sigma-Aldrich (St. Louis, Mo.); 1-ethyl-3-[3-dimethylaminopropyl]-carbodiimide hydrochloride (EDC), N-hydroxysulfosuccinimide (Sulfo-NHS), and Sulfosuccinimidyl-4-(N-maleimidomethyl)-cyclohexane-1-carboxylate (sulfo-SMCC) obtained from Pierce (Rockford, Ill.); and bsp-RGD(15).

The thermoreversible IPN coatings are polymerized sequentially. First an NIPAAm/mPEG200MA layer is polymerized directly on quartz discs, subsequently a poly(ethylene glycol/acrylic acid) (pEG/AAc) layer is polymerized within the NIPAAm/mPEG200MA network, but not crosslinked to it. The IPNs are then modified with bsp-RGD(15) to promote for stem cell attachment. Quartz discs (1″ O.D.×¼″ thick; Chemglass, Inc) are cleaned with an oxygen plasma (March Plasmod; Concord, Calif.) for 5 min at 1 Torr. The discs are functionalized with an organosilane, MPMS, by immersing in a solution composed of 94% (v/v) MeOH, 5% (v/v) water, 1% (v/v) MPMS, and 1 mM AA solution for 5 minutes and baking for 30 min at 110° C. Solutions of 10% NIPAAm/m PEG200MA/pEG₂₀₀DA [molar ration 96:3:1] are prepared in water and degassed. Polymerization is initiated with AP and TEMED. NIPAAm/mPEG200MA/pEG₂₀₀DA solutions are pipetted onto functionalized quartz discs and sandwiched with top coverslips that are modified with CMS. After polymerization, the samples are immersed in UPW, and top coverslips removed. The second layer of PEG/AAc is polymerized on top of and within the NIPAAm/mPEG200MA layer by previous methods (Harbers, et al., Langmuir, 21(18):8374-8384. (2005)) NIPAAm/mPEG200MA-modified quartz discs are allowed to equilibrate in a solution of 0.02 g/mL PEG1000MA, 0.01 g/mL Bis, 0.3348 g/mL CQ, and AAc in methanol for 5 min. The pEG/AAc layer is polymerized in a light box (Rayonet; Branford, Conn.) for 40 min, and samples are rinsed in methanol and water. The surfaces were then functionalized with a ligand, for example bsp-RGD(15). A PEG spacer is tethered to the AAc sites in the pEG/AAc layer by exposure to a solution of 0.20 g/mL of pEG(NH₂)₂, 0.4 mg/mL EDC, and 1.1 mg/mL Sulfo-NHS for one hour. Next, a heterobifunctional crosslinker, sulfo-SMCC (0.5 mg/mL in sodium borate buffer, pH 7.5, 30 min) is used to attach the ligand (0.1M solution in sodium borate buffer, pH 6.6, reacted overnight). Atomic Force Microscopy Experiments are performed in order to measure the Young's modulus (E) of the thermoreversible IPNs. A Bioscope AFM in force-mode and a fluid cell is used in these experiments. A v-shaped silicon nitride tip is modified with a 10 um polystyrene bead in order to reduce strain on the gels during measurements. The E of the gels can be made to vary between 200 Pa to 100 kPa by either adjusting the concentration of mPEG200MA, mPEG200DA, or both. On these thermoreversible IPNs hESCs are cultured using complete culture medium (KSR) that have been condiditioned by mouse embryonic feeders (MEFs). KSR consists of: Knockout-DMEM (Gibco), 20% Knockout Serum Replacement (Gibco), 2 mM Glutamine (Gibco), 0.1 mM non-essential amino acids (NEAA) (Gibco), 0.1 mM β-Mercaptoethanol (Sigma), and 4 ng/mL basic fibroblast growth factor (FGF)-2 (R&D Systems). KSR is added to irradiated MEFs for 24 hours and removed such that soluble factors from the MEFs are included. Since the thermoreversible IPNs undergoes a LCST transition, whereby the change in the surface's physical properties can release the hESCs from the hydrogel surface, reducing the temperature below the LCST to release the hESCs.

### Example 7

This example details the formation of a sIPN to support stem cell self-renewal or differentiation. The cell-binding adhesion peptide bsp-RGD(15) and the heparin-binding adhesion peptide acetyl-CGGFHRRIKA-NH₂ (-FHRRIKA-), of bone sialoprotein (BSP), were incorporated into the p(NIPAAm-co-AAc)sIPN.

The materials used to synthesize the sIPN include the following: NIPAAm, AAc, N,N′-methylenebisacrylamide (BIS), ammonium peroxydisulfate (AP), N,N,N′,N′-tetramethylethylenediamine (TEMED), and linear p(AAc) chains (450,000 g/mol, acid form), which were obtained from Polysciences, Inc. (Warrington, Pa.), and Dulbecco's Phosphate-Buffered Saline (PBS; 1.51 mM KH₂PO₄, 155 mM NaCl, and 2.7 mM Na₂HPO₄; without CaCl₂, without MgCl₂; pH=7.2±0.1), which was obtained from GIBCO BRL (Grand Island, N.Y.).

The synthesis of the polymeric networks is separated into two parts: first the linear polymer chains are functionalized with a ligand of interest, and purified; subsequently, the sIPN is synthesized with the bio-functionalized linear chains.

7.1 Synthesis of the Bio-Functionalized Linear Chain

The hydrazide end of N-[E-Maleimidocaproic acid]hydrazide (EMCH)(0.02 g/mL) was first reacted with the —COO⁻ groups in the p(AAc) chains (1 mg/mL) using 1-ethyl-3-(3-dimethylaminopropyl) carbodiimide (EDC; Pierce, 0.4 mg/mL) and N-hydroxysulfosuccinimide (Sulfo-NHS, Pierce, 1.1 mg/mL) in 2-(N-morpholino) ethanesulfonic acid, 0.9% NaCl, conjugation buffer (MES, Pierce, 0.1 M, pH 6.5) for 1 hour at 22° C. The unreacted components were removed via dialysis, the product was lyophilized, and then the maleimide end of EMCH was reacted with the thiol groups of the ligand in 0.1 M sodium phosphate buffer (pH 6.6) for 4 hours at 22° C. Again the product was lyophilized, and the functionalized p(AAc) chains were used to synthesize the semi-IPNs, as detailed below. As a specific example, bsp-RGD(15) is grafted to the pAAc chains and is called pAAc-graft-bsp-RGD(15).

7.2 Preparation of the sIPN

The pAAc-graft-bsp-RGD(15) chains (0.001 g to 0.013 g) were added to 2.4395 g (22 mmol) of NIPAAm, 0.005 g (0.0325 mmol) of BIS, 0.0605 g (0.84 mmol) of AAc, and 50 mL of PBS, and the mixture was bubbled with dry nitrogen gas in a two-neck flask for 15 minutes to remove dissolved oxygen. Following the nitrogen gas purge, 0.020 g (0.0876 mmol) of AP and 200 μL (1.3 mmol) of TEMED were added as the initiator and accelerator, respectively. The mixture was stirred vigorously for 15 s and allowed to polymerize at 22° C. for 19 h under regular fluorescent lighting in a 250 mL glass beaker covered with a glass plate. Following the polymerization, the p(NIPAAm-co-AAc)-based semi-IPN was washed three times, 15-20 minutes each, in excess water to remove unreacted compounds.

### Example 8

sIPN of p(NIPAAm-co-EG200) Cross-Linked by PEG200DA and Interpenetrated by Peptide-Functionalized Hyaluronic Acid

The materials used to synthesize the sIPN include N-isopropyl acrylamide (NIPAAm), methoxy poly(ethylene glycol) (MW=200) methacrylate (mPEG200MA) (MW=300 g/mol), poly(ethylene glycol) (MW=200) diacrylate (PEG200DA) (MW=302 g/mol), ammonium peroxydisulfate (AP), and N,N,N′,N′-tetramethylethylenediamine (TEMED) obtained from Polysciences, Inc. (Warrington, Pa.), as well as incomplete Dulbecco's Phosphate-Buffered Saline (iPBS; 1.51 mM KH₂PO₄, 155 mM NaCl, and 2.7 mM Na₂HPO₄; without CaCl₂, without MgCl₂; pH=7.2±0.1), which was obtained from GIBCO BRL (Grand Island, N.Y.).

The hydrazide end of EMCH (0.02 g/mL) was first reacted with the —COO⁻ groups in the hyaluronic acid (HyA) chains (1 mg/mL) using 1-ethyl-3-(3-dimethylaminopropyl) carbodiimide (EDC; Pierce, 0.4 mg/mL) and N-hydroxysulfosuccinimide (Sulfo-NHS, Pierce, 1.1 mg/mL) in 2-(N-morpholino) ethanesulfonic acid, 0.9% NaCl, conjugation buffer (MES, Pierce, 0.1 M, pH 6.5) for 1 hour at 22° C. The unreacted components were removed via dialysis, the product was lyophilized, and then the maleimide end of EMCH was reacted with the —SH groups of bsp-RGD(15) in 0.1 M sodium phosphate buffer (pH 6.6) for 4 hours at 22° C. The product was lyophilized, and the functionalized HyA chains were used to synthesize the semi-IPNs, as detailed below.

The functionalized HyA (25 mg) was dissolved in 15 mL iPBS along with 5% w/v total of NIPAAm, mPEG200MA, and PEG200DA, followed by bubbling the solution with dry nitrogen gas for 30 minutes to remove dissolved oxygen. Following the nitrogen purge, 279 uL of 10% w/v AP (27.9 mg, 0.122 mmol) and 183 uL TEMED (142 mg, 1.22 mmol) were added as the initiator and accelerator, respectively, to the solution, which was then gently mixed. The monomer solution was allowed to polymerize at room temperature for 18 hours under a dry nitrogen atmosphere. The sample sIPN hydrogel compositions and properties are listed in Table 2 below.

TABLE 2
Example 8 sample sIPN compositions

| | NIPAAm | PEG200DA | mPEG200MA | | | |
|---|---|---|---|---|---|---|
| | mol % | mol % | mol % | 22C G* (Pa) | 37C G* (Pa) | LCST (C) |
| Sample 8A | 98.7 | 1.0 | 0.3 | 68.6 | 1970 | 32.9 |
| Sample 8B | 98.4 | 1.0 | 0.6 | 64.4 | 32300 | 32.9 |
| Sample 8C | 96.1 | 1.0 | 2.9 | 44.1 | 91500 | 33.6 |

### Example 9

Hydrolytically-Degradable sIPN of p(NIPAAm-co-AAc) Interpenetrated by Peptide-Functionalized Linear HyA

This example defines a p(NIPAAm-co-AAc) sIPN with a hydrolytically cleavable crosslinker. The water-soluble crosslinker was a telechelic molecule composed of poly(ethylene glycol) (PEG) flanked at both ends with either poly(lactide) (PL), poly(ε-caprolactone) (PEC), or a copolymer of each. The ends of the chain were acrylated using acryloyl chloride and triethylamine (TEA) as described for the enzymatically degradable crosslinker. In one synthesis, the average molecular weight of the crosslinker was approximately 8000 g/mol, and the molar ratio of the PEG, PL and PEC was 1:5:0.5. The materials used to synthesize the sIPN include NIPAAm, AAc, ammonium peroxydisulfate (AP), and N,N,N′,N′-tetramethylethylenediamine (TEMED) obtained from Polysciences, Inc. (Warrington, Pa.), as well as incomplete Dulbecco's Phosphate-Buffered Saline (iPBS; 1.51 mM KH₂PO₄, 155 mM NaCl, and 2.7 mM Na₂HPO₄; without CaCl₂, without MgCl₂; pH=7.2±0.1), which was obtained from GIBCO BRL (Grand Island, N.Y.). NIPAAm (96 mol %), AAc (2 mol %), and the crosslinker (2 mol %) were polymerized in iPBS in the presence of bio-functionalized HyA chains (see, Example 8) for 19 hours at RT. This sIPN degrades in approximately 15-25 days.

### Example 10

Hydrolytically-Degradable sIPN of p(NIPAAm-co-EG200) Interpenetrated by Peptide-Functionalized Linear pAAc.

This example defines a sIPN of p(NIPAAm-co-EG200) with a hydrolytically cleavable crosslinker. The water-soluble crosslinker was a telechelic molecule composed of poly(ethylene glycol) (PEG) flanked at both ends with either poly(lactide) (PL), poly(ε-caprolactone) (PEC), or a copolymer of each. The ends of the chain were acrylated using acryloyl chloride and triethylamine (TEA) as described for the enzymatically degradable crosslinker. The materials used to synthesize the sIPN include NIPAAm, methoxy poly(ethylene glycol) (MW=200) methacrylate (mPEG200MA) (MW=300 g/mol), ammonium peroxydisulfate, and N,N,N′,N′-tetramethylethylenediamine obtained from Polysciences, Inc. (Warrington, Pa.), as well as incomplete Dulbecco's Phosphate-Buffered Saline (iPBS; 1.51 mM KH₂PO₄, 155 mM NaCl, and 2.7 mM Na₂HPO₄; without CaCl₂, without MgCl₂; pH=7.2±0.1), which was obtained from GIBCO BRL (Grand Island, N.Y.). NIPAAm (96 mol %), mPEG200MA (3 mol %), and the crosslinker (1 mol %) were polymerized in iPBS in the presence of bio-functionalized pAAc chains (see, Example 7) for 19 hours at RT.

### Example 11

Hydrolytically-Degradable sIPN of p(NIPAAm-co-EG200) Interpenetrated by Peptide-Functionalized Hyaluronic Acid (HyA).

This example defines a sIPN of p(NIPAAm-co-EG200) with a hydrolytically cleavable crosslinker. The water-soluble crosslinker was a telechelic molecule composed of poly(ethylene glycol) (PEG) flanked at both ends with either poly(lactide) (PL), poly(ε-caprolactone) (PEC), or a copolymer of each. The ends of the chain were acrylated using acryloyl chloride and triethylamine (TEA) as described for the enzymatically degradable crosslinker. The materials used to synthesize the sIPN include NIPAAm, methoxy poly(ethylene glycol) (MW=200) methacrylate (mPEG200MA) (MW=300 g/mol), ammonium peroxydisulfate, and N,N,N′,N′-tetramethylethylenediamine obtained from Polysciences, Inc. (Warrington, Pa.), as well as incomplete Dulbecco's Phosphate-Buffered Saline (iPBS; 1.51 mM KH₂PO₄, 155 mM NaCl, and 2.7 mM Na₂HPO₄; without CaCl₂, without MgCl₂; pH=7.2±0.1), which was obtained from GIBCO BRL (Grand Island, N.Y.). Grafting of biomolecules to HyA chains was achieved in the following manner. The hydrazide end of EMCH (0.02 g/mL) was first reacted with the —COO⁻ groups in the HyA chains (1 mg/mL) using 1-ethyl-3-(3-dimethylaminopropyl) carbodiimide (EDC; Pierce, 0.4 mg/mL) and N-hydroxysulfosuccinimide (Sulfo-NHS, Pierce, 1.1 mg/mL) in 2-(N-morpholino) ethanesulfonic acid, 0.9% NaCl, conjugation buffer (MES, Pierce, 0.1 M, pH 6.5) for 1 hour at 22° C. The unreacted components were removed via dialysis, the product was lyophilized, and then the maleimide end of EMCH was reacted with the —SH groups of the bsp-RGD(15) in 0.1 M sodium phosphate buffer (pH 6.6) for 4 hours at 22° C. These functionalized chains are termed HyA-graft-bsp-RGD(15). The product was lyophilized, and the functionalized HyA chains were used to synthesize the semi-IPNs, as detailed below. The HyA-graft-bsp-RGD(15) (125 mg) was dissolved in 50 mL iPBS along with 2.194 g NIPAAm (19.4 mmol), 0.306 g mPEG200MA (1.02 mmol), and the hydrolytically-degradable crosslinker (1 mol %), followed by bubbling the solution with dry nitrogen gas for 30 minutes to remove dissolved oxygen. Following the nitrogen purge, 279 uL of 10% w/v AP (27.9 mg, 0.122 mmol) and 183 uL TEMED (142 mg, 1.22 mmol) were added as the initiator and accelerator, respectively, to the solution, which was then gently mixed. The monomer solution was allowed to polymerize at room temperature for 18 hours under a dry nitrogen atmosphere.

### Example 12

sIPN of Hyaluronic Acid Graft EMCH using Dithiol Crosslinkers Interpenetrated by Peptide-Functionalized Hyaluronic Acid

Linear HyA chains were activated for crosslinking in the following manner. The hydrazide end of EMCH (0.02 g/mL) was reacted with the —COO⁻ groups in the HyA chains (1 mg/mL) using 1-ethyl-3-(3-dimethylaminopropyl) carbodiimide (EDC; Pierce, 0.4 mg/mL) and N-hydroxysulfosuccinimide (Sulfo-NHS, Pierce, 1.1 mg/mL) in 2-(N-morpholino) ethanesulfonic acid, 0.9% NaCl, conjugation buffer (MES, Pierce, 0.1 M, pH 6.5) for 1 hour at 22° C. The unreacted components were removed via dialysis, the product was lyophilized. These HyA chains with maleimide terminated grafts of EMCH can be reacted with any dithiol containting molecule to generate a crosslinked network. When the network is crosslinked in the presence of a linear biofunctionalized chain, i.e. HyA, a sIPN is formed. Specifically, di-thiol poly(ethylene glycol) (MW 3400) (Nektar, Huntsville, Ala.) and biofunctionalized HyA were combined at final concentrations ranging from 1 to 33 mg/mL to the maleimide activated HyA chain solution. Gelation rates depend on the range of crosslinker concentrations and can be as short as 10 mins. By modulating the amount of crosslinker (i.e., either the concentration of the dithiol molecule or degree of grafting of the HyA chain), the mechanical properties of the sIPN can be tuned.

### Example 13

Maintenance of hESCs on sIPNs of (p(NIPAAm-co-AAc) with Enzymatically-Degradable Crosslinks

In this example, hESCs were grown on a sIPN consisting of loosely crosslinked poly(N-isopropylacrylamide-co-acrylic acid) (p(NIPAAm-co-AAc)). The p(NIPAAm-co-AAc) was crosslinked with an acrylated peptide (QPQGLAK-NH₂), a sequence designed to be cleaved by matrix metalloproteinase-13 (MMP-13) and other collagenases. A sIPN was synthesized by the addition of p(AAc)-graft-bsp-RGD(15), to provide cell binding domains, during the polymerization of p(NIPAAm-co-AAc). An important feature of this sIPN is that the gel stiffness is tunable by varying the concentration of: (a) the crosslinker, and (b) of the linear p(AAc)-graft-bsp-RGD(15) chains.

Protease-labile crosslinkers not only contribute to the overall mechanical properties of the sIPN, but they also affect the degradation rate. The Gln-Pro-Gln-Gly-Leu-Ala-Lys (QPQGLAK) diacrylate used as a peptide crosslinker was designed to enable the cell-mediated proteolytic remodeling to occur within the sIPNs. Michaelis-Menten parameters, Kₘ and k_{cat}, were determined for the cleavage of candidate peptide crosslinker in solution by activated human recombinant MMP-13 and a general collagenase from Clostridium histolyticum by using an HPLC peak area detection protocol (Table 4). Within the timeframe measured, Lineweaver-Burk plots were linear and therefore obeyed Michaelis-Menten conditions for the concentrations studied.

TABLE 4
The digestion kinetics of QPQGLAK by recombinant human (rh) MMP-13 and C. histolyticum
collagenase in our studies were measured by HPLC and compared to the digestion of other
peptide substrates by MMP-13 (Lauer-Fields et al., J. Biol. Chem., 275(18): 13282-90 (2000);
(Mitchell, et al., J. Clin. Invest., 97(3): 761-8. (1996); (Deng, et al., Journal Of Biological
Chemistry, 275(40): 31422-31427 (2000)). The cleavage site is between amino acids
P₁ and P₁′. The selectivity of MMP-13 for the substrates is indicated by
comparing k_{cat}/Kₘ for MMP-13 with other MMPs. The sequences taken from
literature studies were determined from phage display studies (Deng, et al., Journal Of
Biological Chemistry, 275(40): 31422-31427 (2000)).

| | | | | | | | | | | | Selectivity (k_{cat}/Kₘ ratio for | | |
|---|---|---|---|---|---|---|---|---|---|---|---|---|---|
| | | Substrate | | | | | | | | k_{cat}/Kₘ | MMP-13 to MMP-x) | | |
| Name | Enzyme | P₄ | P₃ | P₂ | P₁ | P₁′ | P₂′ | P₃′ | P₄′ | (s⁻¹M⁻¹) | MMP-1 | MMP-9 | MMP-3 |
| Coll II- | rh MMP-13 | Q | P | Q | G | L | A | K | | 729 | | | |
| H1 | | | | | | | | | | | | | |
| Coll II- | collagenase | Q | P | Q | G | L | A | K | | 32 | | | |
| H1 | | | | | | | | | | | | | |
| CP | rh MMP-13[3] | G | P | L | G | M | R | G | L | 4.22 × 10⁶ | 820 | 11 | 1300 |
| C2-22 | rh MMP-13[3] | G | P | R | P | F | N | Y | L | 1.08 × 10⁶ | 180 | 21 | 7.9 |
| C5-27 | rh MMP-13[3^{]} | G | P | F | G | F | K | S | L | 5.11 × 10⁵ | 2900 | 4.8 | 250 |
| C2- | rh MMP-13[3] | G | A | L | G | L | S | L | | 3.53 × 10⁴ | 8.3 | 4.6 | 14 |
| 12P3A | | | | | | | | | | | | | |
| C3-16 | rh MMP-13[3^{]} | G | P | K | G | V | Y | S | L | 1.6 × 10⁶ | 5500 | 2.2 | 3600 |
| Coll II | rh MMP-13[2] | G | P | Q | G | L | A | G | Q | 3194 | | | |

| | | |
|---|---|---|
| rh MMP-13[1] | Synthetic triple helical peptide | 3293 |

1. (Lauer-Fields et al., J. Biol. Chem., 275(18): 13282-90 (2000))
2. (Mitchell, et al., J. Clin. Invest., 97(3): 761-8. (1996))
3. (Deng, et al., Journal Of Biological Chemistry, 275(40): 31422-31427 (2000))

The degradation rate of the sIPNs can be adjusted by using alternative peptide crosslinkers with higher k_{cat}/Kₘ ratios (Table 4), [3]. In addition, sIPNs can be constructed with more than one type of peptide crosslinker (each with a different protease degradation rate) to generate heterogeneously degrading sIPNs. A variety of peptide based MMP substrates can be chosen from to control the degradation rate of a cross linked sIPN, allowing for matching the rate of hydrogel degradation to the local biological application. We have chosen three sequences that will allow for a slow, moderate, and fast degradation by MMP-13 with specificity over other collagenases, MMP-2 and MMP-9. The first peptide crosslinker, allowing for a slow rate of MMP-13 cleavage, is a 6 amino acid sequence (QPQGLAK) suitable for acrylation and incorporation into a polymer network by free radical polymerization. The second and third peptide sequences listed (GPLGLSLGK and GPLGMHGK), based on sequences in Table 4, have been selected as also being suitable for acrylation and polymerization, as well for faster cleavage rates by MMP-13 activity.

Polymerization follows that outlined in Example 7 with the exception that BIS is replaced by the peptide crosslinker. For a p(NIPAAm-co-AAc) crosslinked with QPQGLAK, the LCST phase transition was determined using an UV-vis spectrophotometer by monitoring the transmittance of visible light (λ=500 nm) as a function of temperature. The sIPN undergoes a LCST at 35° C. The mechanical and viscoelastic properties of the sIPNs were characterized by dynamic oscillatory shear measurements, using a parallel plate rheometer (Paar Physica MCR 300). Rheological measurements were performed over a frequency range of 0.001 Hz-10 Hz to determine the complex modulus (G*) and loss angle. The mean G* at 22° C. at 1 Hz was 77.4 Pa±30.3 (SE), and at 37° C. at 1 Hz was 129.1 Pa±61.6 (SE). The sIPN was polymerized in 12-well plates and sterilized by the use of ethanol. hESCs were cultured on the sIPN surface and optimal hESC culture conditions were used. Complete culture medium (KSR) consisted of: Knockout-DMEM (Gibco), 20% Knockout Serum Replacement (Gibco), 2 mM Glutamine (Gibco), 0.1 mM non-essential amino acids (NEAA) (Gibco), 0.1 mM β-Mercaptoethanol (Sigma), and 4 ng/mL basic fibroblast growth factor (FGF)-2 (R&D Systems). On the sIPNs, hESCs are cultured using MEF-conditioned KSR. hESCs were evaluated by morphology, live/dead stain (calcein AM and Ethidium Homodimer), and immunofluorescence against the Oct-4 transcription factor, a highly specific and necessary hESC marker and SSEA-4, a cell surface marker for hESCS. The sIPN was able to support short-term hESC self-renewal in the absence of a mouse or human feeder layer. hESCs were cultured on sIPN of four RGD adhesion ligand concentrations of 0, 45, 105, 150 μM (FIG. 9). The hESC colonies were morphologically intact and live/dead stain indicated a combination of living and dead cells. Finally, immunofluorescence revealed positive Oct-4 and SSEA-4 expression in the hESC colonies (FIGS. 10 and 11), an indication the hESCs retained their undifferentiated state.

### Example 14

To assess cell proliferation on sIPNs with different complex shear moduli (G*) and bsp-RGD(15) ligand concentration a series of protease-degradable sIPNs were synthesized while modulating the bsp-RGD(15) concentration and G* (measured at 1 Hz at 37° C.). In 96 well plates, sIPNs were sterilized in 70% ethanol and washed 3 times with PBS at 37° C. Cells isolated from newborn rat calvaria were seeded onto the surface of each sIPN at a surface density of 6000 cells/cm² and maintained with DMEM supplemented with 15% FBS, 1 mM sodium pyruvate, 5 g/ml ascorbic acid, 150 nM dexamethasone, 1% fungizone and 1% penicillin-streptomycin. Cell density was quantified with the WST-1 cell proliferation reagent after 5 days in culture. Cell proliferation data were plotted as a function of bsp-RGD(15) concentration and G*, and were fit using a least squares regression with JMP(SAS) software (Cary, N.C.), (FIG. 8). Significant effects of RGD concentration (p<0.05) and G* (p<0.05) were observed. The 2D contour plot identifies lines of constant proliferation (cells/area) based on the independent variable or factors bsp-RGD(15) concentration and G*. The shaded region in the 2D contour plot represents zero cells/cm², thus factor combinations in this region don't support cell proliferation and may induce apoptosis. An interaction effect is evident from both plots and suggests the ligand is active in the sIPNs, even after radical polymerization.

### Example 15

Method for Stem Cell Recovery with using Enzymes for Enzymatically Degradable sIPNs

This example describes a method for harvesting hESC grown on enzymatically-crosslinked sIPNs. Human ESCs can be grown on thermoreversible and enzymatically-degradable sIPNs as defined in Example 13. Enzymatically degradable sIPNs were polymerized in 6-well plates and sterilized by the use of ethanol. The hESCs were cultured on the sIPNs using MEF-conditioned complete culture medium (KSR) consisting of: Knockout-DMEM (Gibco), 20% Knockout Serum Replacement (Gibco), 2 mM Glutamine (Gibco), 0.1 mM non-essential amino acids (NEAA) (Gibco), 0.1 mM β-Mercaptoethanol (Sigma), and 4 ng/mL basic fibroblast growth factor (FGF)-2 (R&D Systems). The hESCs can be harvested by using MMP enzymes to degrade the enzymatically-degradable crosslinks. Enzymes are added to the culture system for 30-40 minutes to degrade the edsIPN sIPN and release the hESCs.

### Example 16

This is an example of a novel method to harvest hESCs from a sIPN culture surface. Currently, hESCs are detached from the culture surface (feeder layer/matrigel) using collagenase and other enzymes. These enzymes are derived from animal products, which raise concerns about disease transmission. The sIPN system offers two novel methods for detachment and retrieval of hESCs. First, the sIPN undergoes a LCST whereby the change in volume can disrupt the cell adhesion to the material and release the hESCs from the sIPN surface. In this case, hESCs are cultured on the sIPN at 37° C. The culture system is then placed in a environment below the LCST temperature for the sIPN for 10-30 minutes to retrieve the hESCs. Since the sIPN undergoes a LCST transition, whereby the change in volume can release the hESCs from the sIPN surface, reducing the temperature below the LCST releases the hESCs from the substrate. Cells are then collected.

### Example 17

Neural Cells on sIPN

In this example, rat adult neural stem cells were grown on a sIPN consisting of loosely crosslinked poly(N-isopropylacrylamide-co-acrylic acid) (p(NIPAAm-co-AAc)). The p(NIPAAm-co-AAc) was crosslinked with an acrylated peptide (QPQGLAK-NH₂), a sequence designed to be cleaved by matrix metalloproteinase-13 (MMP-13) and other collagenases. In addition, a semi-interpenetrating polymer network was synthesized by the addition of 60 μM polyacrylic acid-graft-bsp-RGD (15), to provide cell binding domains, during the polymerization of p(NIPAAm-co-AAc). An important feature of this sIPN is that the gel stiffness is tunable by varying the concentration of: (a) the crosslinker, and (b) of the linear p(AAc)-graft-bsp-RGD (15) chains. The sIPN undergoes a lower critical solution temperature (LCST) at ˜32-35° C. Rheological measurements were performed over a frequency range of 0.001 Hz-10 Hz to determine the complex modulus (G*) and loss angle. The mean G* at 22° C. at 1 Hz was 24.40 Pa±2.0 (SD), and at 37° C. at 1 Hz was 87.40 Pa±2.1 (SD). The sIPN was polymerized in 96-well plates and sterilized by the use of ethanol.

NSCs were cultured on the sIPN surface under conditions listed in Example 4, either in 20 ng.ml⁻¹ basic fibroblast growth factor (bFGF) for cell proliferation or 1 μM retinoic acid with 5 μM forskolin for neuronal differentiation. NSCs were evaluated by morphology and a live/dead stain (calcein AM and Ethidium Homodimer, Molecular Probes, Eugene, Oreg.). After 15 days, the sIPN was able to support NSC self-renewal with few necrotic cells (FIG. 12a). In contrast, NSC were not able to differentiate well within the sIPN, as evidenced by a large percentage of necrotic cells (FIG. 12b). Thus, this example defines an alternative embodiment for conditions for self-renewal of NSCs, but not differentiation of these cells. This example also demonstrates the sensitivity of NSC to differentiation conditions is modulus dependent.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An interpenetrating polymer network comprising:
(a) a first cross-linked polymer; and
(b) a second cross-linked polymer entangled within said first cross-linked polymer
wherein a member selected from said first cross-linked polymer and said second cross-linked polymer is covalently grafted to a ligand which promotes a member selected from stem cell adhesion to the network, stem cell growth, stem cell proliferation, stem cell self-renewal, stem cell differentiation, and combinations thereof.

2. A semi-interpenetrating polymer network comprising:
(a) a cross-linked polymer; and
(b) a linear polymer entangled within said cross-linked polymer,
wherein said linear polymer is covalently grafted to a ligand which promotes a member selected from stem cell adhesion to the network, stem cell growth, stem cell proliferation, stem cell self-renewal, stem cell differentiation, and combinations thereof.

3. The network according to claims 1 or 2 wherein said ligand is a member selected from amino acids, peptides, peptoids, proteins, nucleic acids, carbohydrates and combinations thereof.

4. The network according to claim 3 wherein said ligand comprises a peptide sequence which is a member selected from RGD, XBBXBX, FHRRIKA, PRRARV, REDV, DEGA, YIGSR, IKVAV, PHSRN, KGD, and cyclic variants thereof
wherein each X is a member independently selected from glycine, alanine, valine, leucine, isoleucine, phenylalanine and proline; and
each B is a member independently selected from lysine, arginine and histidine.

5. The network according to claim 3 wherein said stem cell is a member selected from embryonic stem cells, adult marrow stem cells, adult neural stem cells, cord blood stem cells, adult skin stem cells, adult liver stem cells, adult olfactory stem cells, adult adipose-derived stem cells, adult hair follicle stem cells, adult skeletal muscle stem cells, adult myogenic muscle stem cells, satellite cells, mesenchymal stem cells and neural stem cells.

6. The network according to claim 3 further comprising a stem cell.

7. The network according to claim 3, further comprising a molecule which is non-covalently entangled with the network.

8. The network according to claim 7, wherein said molecule is a member selected from peptides, morphogens, growth factors, hormones, small molecules and cytokines.

9. The network according to claim 8, wherein said molecule is a member selected from adhesion peptides from ECM molecules, laminin peptides, heparin sulfate proteoglycan binding peptides, heparan sulfate proteoglycan binding peptides, Hedgehog, Sonic Hedgehog, Shh, Wnt, bone morphogeneic proteins, Notch (1-4) ligands, Delta-like ligand 1, 3, and 4, Serrate/Jagged ligands 1 and 2, fibroblast growth factor, epidermal growth factor, platelet derived growth factor, Eph/Ephrin, Insulin, Insulin-like growth factor, vascular endothelial growth factor, neurotrophins, BDNF, NGF, NT-3/4, retinoic acid, forskolin, purmorphamine, dexamethasone, 17β-estradiol and metabolites thereof, 2-methoxyestradiol, cardiogenol, stem cell factor, granulocyte-macrophage colony-stimulating factor, granulocyte colony-stimulating factor, interleukins, IL-6, IL-11, cytokines, Flt3-1, Leukaemia inhibitory factor, transferrin, intercellular adhesion molecules, ICAM-1 (CD54), VCAM, NCAM, tumor necrosis factor alpha, HER-2, and stromal cell-derived factor-1 alpha.

10. The network according to claim 3, wherein a cross link in at least one of the cross-linked polymers in the interpenetrating polymer network or the cross link in the cross-linked polymer of the semi-interpenetrating polymer network is biodegradable.

11. The network according to claim 1 or 2 wherein said cross-linked polymer or said linear polymer is non-fouling.

12. The network according to claim 11 wherein said non-fouling cross-linked polymer or linear polymer comprises a subunit which is a member selected from hyaluronic acid, acrylic acid, ethylene glycol, methacrylic acid, acrylamide, hydroxyethyl methacrylate, mannitol, maltose, taurine, betaine, modified celluloses, hydroxyethyl cellulose, ethyl cellulose, methyl cellulose, hydroxyethyl methyl cellulose, hydroxypropyl methyl cellulose, carboxymethyl cellulose, modified starches, hydrophobically modified starch, hydroxyethyl starch, hydroxypropyl starch, amylose, amylopectin, oxidized starch, and copolymers thereof.

13. A method of optimizing a mechanical property of the network according to claim 1 while maintaining a biochemical property of said network essentially constant, said method comprising:
(a) selecting an optimal value for said mechanical property;
(b) testing said mechanical property of a first said network of claim 1 and obtaining a first value for said mechanical property;
(c) testing said mechanical property of a Xth said network of claim 1 and obtaining a Xth value for said mechanical property,
(d) repeating step (c) until said Xth value for said mechanical property is essentially the same as said optimal mechanical value,
thereby optimizing the mechanical property of the network.
